(19) United States
(12) Patent Application Publication
(10) Pub. No.: US 2023/0135366 A1
(43) Pub. Date: May 4, 2023

(54) LIPOIC ACID CHOLINE ESTER COMPOSITIONS AND METHODS TO STABILIZE INTO PHARMACEUTICALLY RELEVANT DRUG PRODUCTS

(71) Applicant: NOVARTIS AG, Basel (CH)

(72) Inventors: Shikha BARMAN, Bedford, MA (US); Koushik BARMAN, Bedford, MA (US); William BURNS, North Richland Hills, TX (US); Kathryn CRAWFORD, Andover, MA (US); Anne-Maria CROMWICK, Saugus, MA (US); Kevin WARD, Arlington, MA (US)

(21) Appl. No.: 16/335,732

(22) PCT Filed: Sep. 22, 2017
(86) PCT No.: PCT/IB2017/055775

§ 371 Date: Mar. 22, 2019

Related U.S. Application Data
(60) Provisional application No. 62/398,748, filed on Sep. 23, 2016.

(51) Int. Cl. A61K 31/385; A61K 47/40; A61K 9/08; A61K 47/18; A61K 9/00

(52) U.S. Cl. CPC A61K 31/385; A61K 47/40; A61K 9/08; A61K 47/183; A61K 9/0048; A61K 9/0019

(57) ABSTRACT

The present invention describes ophthalmic lipoic acid choline ester compositions and specific processes to produce biocompatible formulations of said compositions suitable for the eye.

### FIELD OF THE INVENTION

The present invention generally relates to pharmaceutically-compliant compositions comprising lipoic acid choline ester and specific compositions and methods to stabilize the compositions and minimize irritation to ocular tissue when applied as eye-drops. The compositions herein are contemplated as therapies for (but not limited to) ocular disorders such as presbyopia, dry eye, cataracts, and age-related macular degeneration.

### BACKGROUND OF THE INVENTION

Lipoic acid choline ester (LACE) is a chemically synthesized derivative of R-α-Lipoic Acid.

Lipoic acid, also known as thioctic acid, is an eight carbon fatty acid with a disulfide linkage joining the carbons 6 and 8 to form an 1, 2-dithiolane ring. The acid forms optical isomers of which the isomer R-α-lipoic acid is the most biologically active.

Lipoic Acid Choline Ester (LACE, chemical structure, see FIG. 1) was designed to permeate biological membranes with the incorporation of the cationic choline head group. While lipoic acid does not permeate the cornea, the choline ester derivative of lipoic acid permeates the cornea, is hydrolyzed by corneal esterases and is transformed into the biologically active lipoic acid. LACE has been formulated into an ophthalmic solution to be applied twice daily as an eye-drop to treat presbyopia.

LACE, which is a prodrug consisting of lipoic acid and choline, is a unique molecule to treat presbyopia. Lipoic acid (LA) is the active ingredient and the choline head group serves to aid permeability into the eye. The bonds between LA and choline are hydrolyzed by esterases in the tear film and cornea after the eye drop is administered. The free lipoic acid enters the eye and ultimately reaches the lens. There it is reduced to dihydrolipoic acid by endogenous oxidoreductases which then cause hydrolysis of the cytosolic proteins within the superficial elongated lenticular cells. This protein cleavage allows a free flow of cytosol and reversal of the oxidative processes associated with the age-related stiffening of the lens. It is expected that ophthalmic solutions prepared from LACE will enable accommodation and improve near vision focus in persons with presbyopia, the age-related loss of accommodation.

Presbyopia is an age-related inability to focus on near objects; this condition is caused by physiological changes in the microstructure of the lens resulting in loss of flexibility in the auto-adjustment of focal length and curvature of the lens to bring the visual object under focus. This condition is corrected by corrective lenses. It has been reported that lipoic acid choline ester (“LACE”) (see e.g., U.S. Pat. No. 8,410,462) can restore near vision.

Supporting this claim are ex-vivo studies that demonstrated that lens softening can be induced pharmacologically in human donor lenses using the protein disulfide reducing agent dithiothreitol (DTT), and in mouse lenses with lipoic acid.

This mechanism of action allows the contemplation of treatment of multiple ocular diseases and disorders. These disorders are, but not limited to, presbyopia, age-related macular degeneration, cataract and dry eye.

An issue that has rendered formulation of LACE problematic has been the propensity to destabilize by ring-opening of the dithiolane linkage to form oxidized species that compromise the activity of the molecule. At room temperature, LACE rapidly degrades into oxidized species (See “HPLC Chromatogram of LACE Ophthalmic Solution with Degradation Products”, see FIG. 2). Even when stored at refrigerated temperatures, rapid oxidation occurs in storage as early as 1 week, comprising the utility of the molecule as a drug product. For LACE. Ophthalmic Solution (also referred to as EV06 Ophthalmic Solution) to be utilized in its fullest potential as a drug product, it was critical that the aqueous formulation be stabilized in storage and during use.

Another issue that confounded the pharmaceutical development of LACE ophthalmic solutions was incidences of ocular surface irritation observed in-vivo in a rabbit irritation model. The invention details unexpected parameters that contributed to, or caused ocular irritation and processes to eliminate or minimize these parameters. These parameters were not related to the formulation composition or properties of the drug substance, factors that normally correlated or attributed to ocular irritation.

The compositions and methods described within describe formulations and methods to stabilize ophthalmic LACE formulations long-term.

Also described arc unanticipated discoveries as to the cause of irritation of LACE formulations formulated under certain process conditions, The cause of irritation was correlated to aggregation of LACE salt molecules in water, as part of hydrophobic interactions with surrounding water molecules and ionic interactions with the counter- anion (chloride or iodide). Critical process parameters were identified as key factors in the generation of final, comfortable ophthalmic solutions of LACE Chloride (EV06 Ophthalmic Solution). For the chloride salt, the final process conditions minimized the formation of the degradation species and minimized the formation of species that were attributed to ocular irritation.

With LACE-Iodide, simple process optimization did not generate comfortable solutions. The aggregated species of LACE could not be dispersed when the salt form was iodide, due to the stabilization of the aggregated species by the larger iodide ion.

Once dissolved in an aqueous solution, For LACE-Iodide salt, the aggregation could not be dispersed once formed, settling upon a thermodynamically stable aggregated species that was approximately 39-41% of the LACE-Iodide peak. Correlations were made for associative species and ocular irritation. The second aspect of the invention is stabilization of a LACE Iodide drug product by generating inclusion complexes in cyclodextrins.

### BRIEF SUMMARY OF THE INVENTION

The proposed invention achieves two primary objectives: (a) to generate ophthalmic solutions of LACE that are stable for at least a year at refrigerated storage temperatures of 2-5° C., and (h) to generate formulations (both LACE-Chloride and LACE-Iodide) that are non-irritating to the eye.

The chemical structure of LACE dictates two points of degradation. One is ring opening of the diothiolane ring and the other is oxidative and hydrolytic degradation, As mentioned earlier, LACE interacts with oxygen to rapidly generate oxidized species, in water, LACE is also susceptible to hydrolysis of the ester linkage to generate Lipoic Acid and Choline. The rate at which hydrolysis occurs is correlated to temperature; hydrolysis is less at lower temperatures and pH.

Studies were performed on LACE ophthalmic solution derivatives, also called EV06 Ophthalmic Solution, stored in permeable LOPE eye-dropper bottles, which are gas permeable. Described herein are methods that the inventors have developed to minimize oxidation of the compounded LACE solution during storage.

Additionally, extensive compatibility studies of excipient mixtures with LACE established the criticality of certain excipients as stabilizing factors, the role of pH in stabilization of the hydrolysis of LACE in water, as well as the effect of osmolality-adjusting agents such as sodium chloride and glycerol. Most importantly, the stabilizing effect of Alanine to LACE, as opposed to citrate, phosphate and borate has been described in the proposed invention.

While searching for causes for irritation, it was discovered that LACE, when dissolved in water, forms micelles and micellar aggregates, common to compounds that are amphiphilic in nature. As definition, examples of micelle-forming compounds are phosphatidyl choline, pegylated phosphatidyl choline, PEG-stearate, sorbitol, etc. While the micelle-formation phenomenon of LACE is not unexpected due to the amphiphilic nature of the molecule, the formation of these aggregates at lower temperatures were surprising. The presence of the aggregates was measured by a RP-HPLC method developed in-house. The measurement could be performed both with HPLC-UV and HPLC-ELSD. Both chloride and iodide salts of LACE form micellar aggregates in aqueous solutions, although the LACE iodide forms more stable aggregates in water, due to the stronger interaction of the iodide counter-ion and the cationic LACE molecule. The equilibrium concentration of LACE Iodide aggregates are 39-41% of the API peak. In comparison, the equilibrium concentration of LACE chloride is <1%, after dispersion with agitated stirring.

### A. LACE CHLORIDE IN AQUEOUS SOLUTIONS

LACE chloride aqueous solutions formed gel-like structures at refrigerated temperatures (2-5° C.). It is also expected that the number and aggregation of these micellar assemblies increase with increase in concentration of the micelle-forming drug. The inventors have correlated the extent of micellar aggregation of LACE with ocular surface irritation, a result that was unanticipated and surprising, since micellar vehicles are often contemplated as drug delivery systems for insoluble compounds. Thus, this is the first reported account of irritation correlated to micellar aggregates. Once discovered, this phenomenon needed to be minimized through compounding methods to correlate with comfort.

The formation of micellar aggregates appeared to be correlated to the temperature of compounding (FIG. 4). The formation of self-assemblies is a thermodynamic phenomenon, correlated to efficient lowering of surface free energy to achieve a minimized energy state. When LACE was compounded in water at a lower temperature (5° C.), aggregates that had a gel-like consistency were formed. Compositions formulated at refrigerated temperatures were extremely irritating to the eye. The aggregated state could be quantitated by a RP-HPLC method (see chromatogram shown in FIGS. 12A-12B). A series of investigative experiments demonstrated no presence of polymers or oligomers, when measured by extensive Size Exclusion Chromatography (SEC). Other investigations tested ocular irritation as a function of processes conducted in the presence of ambient air or in the presence of nitrogen. There was no correlation of irritation to air or nitrogen. Both were equally comfortable when formulated at room temperature, although the degradation products were higher in the presence of air. When LACE was compounded at room temperature, the micellar aggregation was lower as quantitated by the HPLC method. LACE compounded at room temperature generated solutions that were comfortable and non-irritating.

Also unanticipated were the “disentangling” of the micellar aggregates. The aggregates formed in LACE aqueous compositions could he “disentangled” as the solutions were left to equilibrate on the benchtop at room temperature, as measured by HPLC. Additional experiments showed that the vigorous mixing achieved de-aggregation. Thus, it was proved that these species were not permanent species with covalent linkages, but rather a self-assembly of LACE aggregates that appeared to have a lower concentration at room temperature, compared to 5° C. LACE aqueous solutions when frozen, formed a stringy consistency. These solutions, when brought up to room temperature and stored at this temperature looked like homogeneous solutions again, lending further credence to concept of temperature dependence of self-assembly.

However, once compounded, aggregate-free solutions of LACE could he stored in refrigerated conditions to minimize oxidative and hydrolytic degradation. it was established through stability studies that the ideal storage temperature of LACE is 2--5° C., to minimize degradation events.

The ideal compounding conditions were determined to be at room temperature (22-25° C.) to yield the least irritating solution and the ideal storage condition was determined to be between 2-5° C., to achieve a stable, comfortable ophthalmic solution of LACE for presbyopia.

To further aid in the stabilization of ophthalmic solutions prepared from LACE, oxygen scavenger packets were placed in mylar, impermeable pouches with the LDPE ophthalmic bottles to prevent oxidation-induced degradation. Extensive stability studies demonstrated achievement of a year's stability of EV06 Ophthalmic Solutions.

Also described in this proposed invention are embodiments of various compositions that stabilize LACE, including other types of aqueous preparations including liposomes, emulsions compounded for the primary purpose of stabilization of the drug.

### B. LACE IODIDE IN AQUEOUS SOLUTIONS

LACE Iodide in aqueous solutions form micellar aggregates (as do LACE Chloride) that cause irritation to ocular tissue. The experiments below describe some of the formulation methods to disrupt micellization.

Tn experiments where Sodium Chloride was either added to an existing LACE-Iodide formulation, or a solution containing Sodium Chloride was used to dissolve the LACE-Iodide API, the “associative species” peak was not significantly decreased.

In experiments where a co-solvent such as Ethanol or Propylene Glycol was used to suspend the API prior to addition of an aqueous vehicle, there was a very significant reduction in the percentage of the associative species. Addition of an organic solvent to an existing formulation also decreased the associative species peak, to a lesser extent.

These results point to formulation strategies that can interfere with the hydrophobic interaction between LACE molecules as a means of controlling the associative species.

### BRIEF DESCRIPTION OF THE DRAWINGS

FIG. 1 illustrates the chemical structure of lipoic acid choline ester (LACE).

FIG. 2 illustrates plots of LACE micellar species at 8.1 minutes at 1, 3 and 4 hours of mixing Formulation KW-LACE-01-86-2.

FIG. 3 illustrates plots of LACE micellar species at 8,1 minutes at 6, 8 and 24 hours of mixing Formulation KW-LACE-01-86-2,

FIG. 4 is a plot illustrating that micellar LACE species are highest when mixed at refrigerated temperatures.

FIG. 5 is a plot illustrating that high micellar LACE concentrations (denoted by large peak between 7,9 and 8.5 minutes on HPLC trace) is correlated to clumped LACE chloride.

FIG. 5B is a plot illustrating that lower micellar LACE concentration is correlated with non-clumped LACE chloride.

FIG. 6 is a plot illustrating the effect of alanine as a function of pH.

FIG. 7 is a plot illustrating the stability of BAC-Tree and glycerol-Tree formulations.

FIG. 8 is a plot illustrating the stability of sulfite-containing formulations.

FIG. 9 is a plot illustrating the stability of BAC-free LACE compositions.

FIG. 110 is a plot illustrating the stability of glycerin-free LACE compositions.

FIG. 11 is a plot illustrating the effect of buffered compositions on LACE stability.

FIG. 12A is a plot illustrating the correlation of irritation score (in a rabbit irritation model) with % LACE micellar species measure by HPLC-UV.

FIG. 12B is a plot illustrating the correlation of irritation score (in a rabbit irritation model) with % LACE miceliar species measure by HPLC-ELSD.

FIG. 12C is a glycerol standard curve.

FIG. 13A is an HPLC plot of FK-LACE-02-15. 1.92% LACE-Iodide (Lot 092309), with 1.8% NaCl added (T=0 hours).

FIG. 13B is an HPLC plot of FK-LACE-02-15, 1.92% LACE-Iodide (Lot 092309), with 1.8% NaCl added (T=4 hours).

FIG. 13C is an HPLC plot of LACE-Iodide (lot 011510), dissolved in pH 4.5 buffer with 1,8% NaCl.

FIG. 14 is an HPLC plot of LACE-Iodide (Lot 011510), dissolved in 78% ethanol.

FIG. 15 is an HPLC plot of LACE-Iodide (Lot 011510), dissolved in 10% propylene glycol.

FIG. 16 is an HPLC plot of LACE Iodide formulated in sulfobutyl ether cyclodextrin.

FIG. 17 is an HPLC plot of LACE iodide formulated with polypropylene glycol to disrupt micellization.

FIG. 18 is a plot illustrating the effect of HP-B-CD on LACE Iodide oxidation,

FIG. 19 is a plot illustrating the effect of FTP-B-CLQ on total impurities of LACE Iodide.

FIG. 20 is a plot comparing LACE-Chloride original formulation and LACE-Iodide HP-B-CD.

FIG. 21 is a calculation of activation energy of oxidized species formation (LACE-Iodide/HP-B-CD versus LACE-Chloride non-HP-B-CD formulation).

FIG. 22 is a Calculation of activation energy of lipoic acid formation (LACE-Iodide/HP-B-CD versus LACE-Chloride non-HP-B-CD formulation).

FIG. 23 is a Franz cell for corneal permeability studies.

FIG. 24 is a permeation of lipoic acid in Study 1 (Corneas 1-3: 1.92% LACE-I with 7.4% FTP-B-CD; Corneas 4-6: 1.5% LACE-Cl, no HP-B-CD).

FIG. 25 is a graph showing the permeation of LACE in Study 1.

FIG. 26 is a graph showing the permeation of LACE in Study 2.

FIG. 27 is a graph illustrating lipoic acid extracted from corneas in Study 2 (Corneas 1-3: 3.0% LACE-iodide formulation; corneas 4-6: 4.5% LACE-iodide formulation).

FIG. 28 is a graph showing the permeation of LACE in Study 3.

FIG. 29 is a graph illustrating lipoic acid extracted from corneas in Study 3 (Corneas 1-3: 3.0% LACE-iodide/HP-B-CD formulation; corneas 4-6: 4.5% LACE-iodide/no HP-B-CD formulation).

FIG. 30 is a graph showing the permeation of LACE in Study 4.

FIG. 31 is a graph illustrating lipoic acid extracted from corneas in Study 4 (Corneas 1-3: 1.92% LACE-iodide/HP-B-CD formulation; corneas 4-6: 1.92% LACE-odide/no HP-B-CD formulation).

FIG. 32 is a plot illustrating change over time in the area percent of associative species as a function of the amount of HP-B-CD in formulation [expressed as mole equivalence (M.E) relative to one mole of LACE].

### DETAILED DESCRIPTION OF THE INVENTION

### A. DEFINITIONS OF TERMS

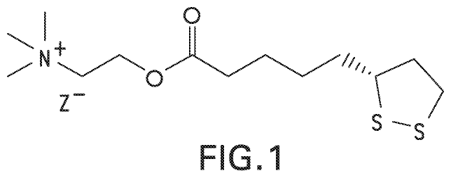

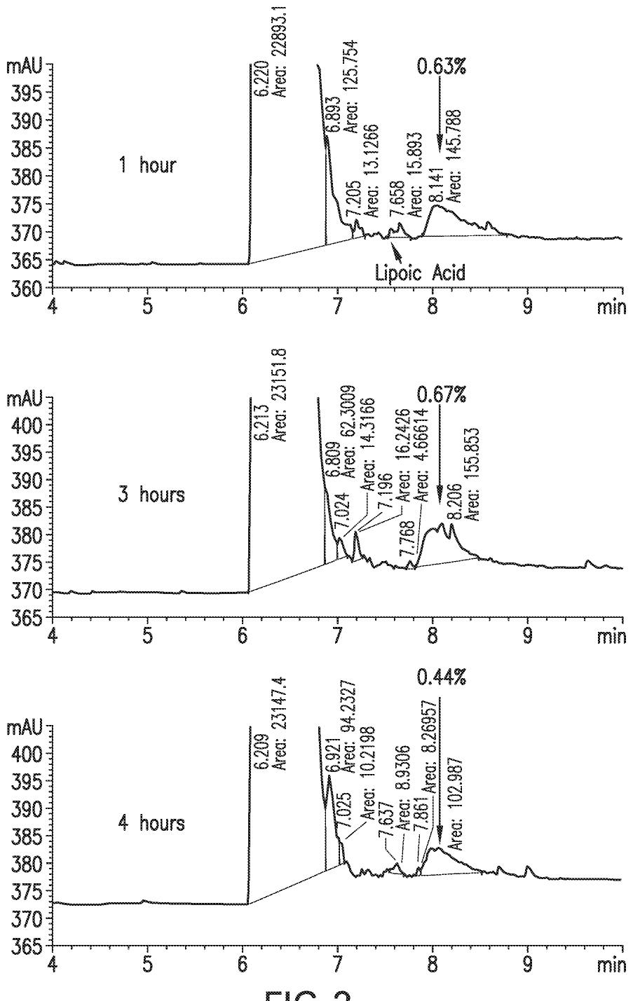

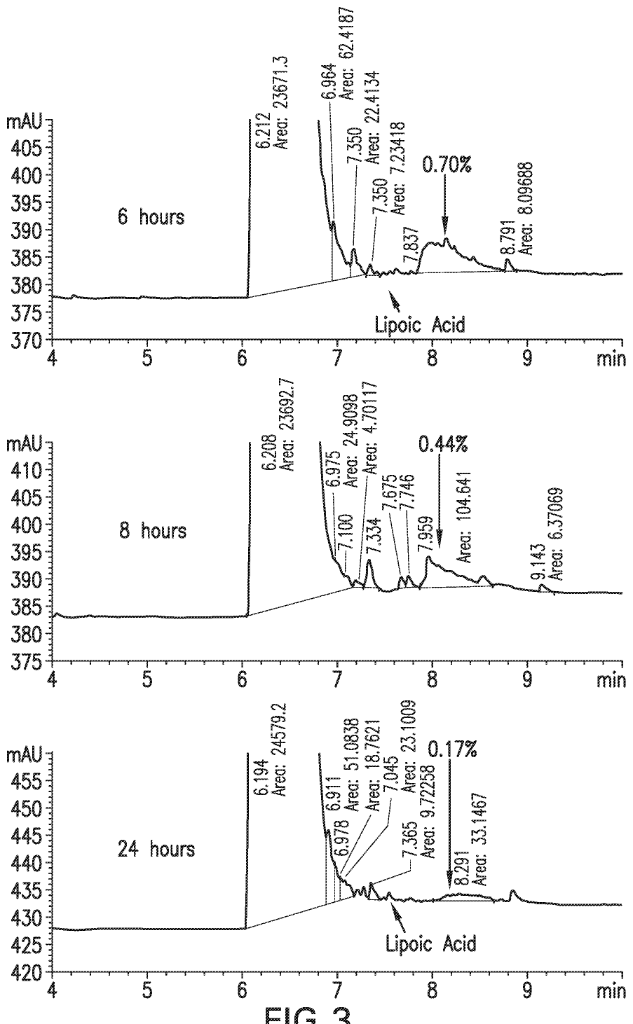

The term “EV06,” “LACE” or “lipoic acid choline ester” is understood to have the following chemical structure as shown in FIG. 1.

As used herein, LACE formulations refer to lipoic acid choline ester formulations. For example, LACE-Chloride 1.5% formulation refers to a formulation having 1.5% lipoic acid choline ester chloride by weight of the formulation. Alternatively, EV06 Ophthalmic Solution, 1.5% refers to a formulation that is comprised of 1.5% lipoic acid choline ester chloride salt. LACE-Iodide 3% refers to a solution that is comprised of 3% LACE-Iodide by weight of the formulation.

As used herein, a “derivative” of lipoic acid choline ester is understood as any compound or a mixture of compounds, excluding lipoic acid and choline, formed from reacting lipoic acid choline ester with a non-aqueous pharmaceutical excipient.

As used herein, the term “self-assembly” denotes a thermodynamic assembling of molecules to achieve the most stable energy state. An example of self-assembly are micelles formed in water, typically formed by molecules with a hydrophobic component and a hydrophilic component. The hydrophilic component of the molecule is on the surface of micelles, while the interior contains the hydrophobic parts; for LACE, the choline head group is on the surface of the micelle.

Unless specifically stated or obvious from context, as used herein, the term “excipient” refers to pharmaceutically acceptable excipient.

The term “treating” refers to administering a therapy in an amount, manner, or mode effective to improve a condition, symptom, or parameter associated with a disease or disorder.

The term “preventing” refers to precluding a patient from getting a disorder, causing a patient to remain free of a disorder for a longer period of time, or halting the progression of a disorder, to either a statistically significant degree or to a degree detectable to one skilled in the art.

The term “therapeutically effective amount” refers to that amount of an active ingredient (e.g., LACE or derivatives thereof), which results in prevention or delay of onset or amelioration of symptoms of an ocular disease or disorder (e.g., presbyopia) in a subject or an attainment of a desired biological outcome, such as improved. accommodative amplitude or another suitable parameter indicating disease state.

As used herein, the term “shelf-stability” or “shelf stable” is understood as a character of or to characterize a composition or an active ingredient (e.g., LACE or derivatives thereof) that is substantially unchanged upon storage. Methods for determining such shelf stability are known, for example, shelf-stability can be measured by HPLC to determine the percentage of the composition or active ingredient (e.g., lipoic acid choline ester) that remains or has been degraded in a formulation following storing the formulation for a certain period of time. For example, shelf stable pharmaceutical composition can refer to a composition, which after being stored as per pharmaceutical standard (ICH) has at least 90% (e.g., 91%, 92%, 93%, 94%, 95%, 96%, 97%, 98%, 99%, or greater than 99%) of the active ingredient lipoic acid choline ester) present in the composition as measured by HPLC.

As used herein, the term “relative retention lime” or “RRT” of a compound can be calculated using the equation “RRT=(t₂−t₀)/(t₁−t₀),” wherein t₀=void time, t₁=retention time of lipoic acid choline ester, and t₂=retention time of the compound, as measured by HPLC.

The term “subject” as used herein generally refers to an animal (e.g., a pet) or human, including healthy human or a patient with certain diseases or disorders (e.g., presbyopia).

### Lace Compositions and Embodiments

As described herein, the proposed invention provides embodiments of pharmaceutical compositions comprising therapeutically effective amounts of lipoic acid choline ester, excipients, buffers and conditions that are compatible and methods and processes that result in biocompatible (non-irritating) and stable solutions suitable as ophthalmic eye-drops.

Concentration of lipoic acid choline ester or derivatives thereof in the pharmaceutical composition can be any concentration from 0.01-0.1%. 0.1% to 10% (e.g., 0.1%, 1%, 2%, 3%, 4%, 5%, 6%, 7%, 8%, 9%, 10%, or any ranges based on these specified numeric values) by weight of the composition, in some embodiments, the concentration of the lipoic acid choline ester in the pharmaceutical composition is 1%. In some embodiments, the concentration of the lipoic acid choline ester in the pharmaceutical composition is 3%. In some embodiments, the concentration of the lipoic acid choline ester in the pharmaceutical composition is 4%. The preferred range of LACE in the composition is 1-3%. Within this range, the preferred composition range is 1,5-5%, The salt form of LACE can be either Iodide or Chloride.

In another embodiment, the effective compositions in the proposed invention are aqueous formulations contain LACE (chloride or iodide) and Alanine, with Alanine at concentrations between 0.1-0.5% , 0.5%-1%, 1%-1.5%, 1.5%-3%, 1.5-5%. Within this range, the preferred composition is 0.5% Alanine and L1.5% LACE. Another preferred embodiment is 0.5% Alanine and 1.5-4% LACE-Iodide or LACE Chloride,

In a preferred embodiment, the effective LACE salt form and Alanine-containing composition contains benzalkonium chloride as a preservative at concentrations between 30-150 ppm.

In another embodiment, the effective LACE. salt form and Alanine-containing drug product composition contains no preservative.

In another embodiment, other preservatives such as polyquartenium, polyhexamethylene Biguanide (PHMB), sofZia is included in the LACE aqueous formulation as preservatives at concentrations approved for human use by the FDA. Other preservatives can be 2-phenyl ethanol, boric acid, disodium edetate,

Since self-assembled micellar solutions of LACE. salt dissolved in water at high concentrations may demonstrate some irritation, a method to render biocompatible solutions may be encapsulation in liposomes. In this case, LACE will be contained in the interior of the liposomes. Liposomes are generally biocompatible with the ocular surface, In another example, LACE salt is encapsulated by complexomg with a cyclodextrin, such as sulfobutylether cyclodextrin or hydroxypropyl beta cyclodextrin.

In another embodiment, the pharmaceutical composition has glycerol in concentrations of 0.1%40%. In a preferred embodiment, the composition has a glycerol concentration of 0.1-5%.

In some embodiments, the preservative is benzalkonium chloride and the biochemical energy source is alanine. In some embodiments, the lipoic acid choline ester has a counter ion selected from the group consisting of chloride, bromide, iodide, sulfate, methanesulfonate, nitrate, maleate, acetate, citrate, fumarate, hydrogen fumarate, tartrate (e.g., (+)-tartrate, (−)-tartrate, or a mixture thereof), bitartrate_{;} succinate, benzoate, and anions of an amino acid such as glutamic acid.

Suitable buffer agent can be any of those known in the art that can achieve a desired pH (e.g., described herein) for the pharmaceutical composition. Non-limiting examples include phosphate buffers (e.g., sodium phosphate monobasic monohydrate, sodium phosphate dibasic anhydrous), acetate buffer, citrate buffer, borate buffers, and HBSS (Hank's Balanced Salt Solution). Suitable amounts of a buffer agent can be readily calculated based on a desired pH. In any of the embodiments described herein, the buffer agent is in an amount that is acceptable as an ophthalmic product. However, in some embodiments, the pharmaceutical composition does not include a buffer agent. In some embodiments, the pH of the aqueous solution or the final pharmaceutical composition is adjusted with an acid (e.g., hydrochloride acid) or a base (e.g., sodium hydroxide) to the desired pH range (e.g., as described herein).

In other embodiments, the buffer system could he selected from borate buffers, phosphate buffers, calcium buffers and combinations and mixtures thereof. In the preferred embodiment, the buffer is an amino acid buffer. In another preferred embodiment, the amino acid buffer is comprised of Alanine.

In some embodiments, the lipoic acid choline ester has a counter ion selected from the group consisting of chloride, bromide, iodide, sulfate, methanesulfonate_{;} nitrate, maleate, acetate, citrate, fumarate, hydrogen fumarate, tartrate, (e.g., (+)-tartrate, (−)-tartrate, or a mixture thereof), succinate, benzoate, and anions of an amino acid such as glutamic acid. Other counter ions are stearate, propionate and furoate.

In some embodiments, the ophthalmic formulation has a pH of 4 to 8, In some embodiments, the ophthalmic formulation has a pH of 4.5. In some embodiments, the ophthalmic formulation comprises at least one ingredient selected from the group consisting of a biochemically acceptable energy source, a preservative, a buffer agent, a tonicity agent, a surfactant, a viscosity modifying agent, and an antioxidant.

In some embodiments, the pharmaceutical composition contains an anti-oxidant. In some preferred embodiments, the anti-oxidant is comprised of ascorbate. In another preferred embodiment, the anti-oxidant contains glutathione. Suitable antioxidant can be any of those known in the art. Non-limiting examples include ascorbic acid, L-ascorbic acid stearate, alphathioglycerin ethylenediaminetetraacetic acid, erythorbic acid, cysteine hydrochloride, N-acetylcystgeine, L-carnitine, citric acid, tocopherol acetate, potassium dichloroisocyanurate, dibutylhydroxytoluene, 2,6-di-t-butyl-4-methylphenol, soybean lecithin, sodium thioglycollate, sodium thiamalate, natural vitamin E, tocopherol, ascorbyl pasthyminate, sodium pyrosulfite, butylhydroxyanisole, 1,3-butylene glycol, pentaerythtyl tetrakis[3-(3,5-di-t-butyl-4-hydroxyphenyl)]propionate, propyl gallate, 2-mercaptobenzimidazole and oxyquinoline sulfate. Suitable amount of antioxidant can be in the range of 0.1% to 5% 0.1%, 0.5%, 1%, 2%, 3%, 4%, 5%, or any ranges based on these specified numeric values) by weight of the composition. In any of the embodiments described herein, the antioxidant is in an amount that is ophthalmically acceptable.

In some embodiments, the pharmaceutical composition is prepared by compounding under an inert environment such as high purity nitrogen or argon. In a preferred embodiment, the pharmaceutical composition is compounded under a nitrogen environment with less than 2 ppm of oxygen.

In some embodiments, the pharmaceutical composition is prepared by compounding at temperatures between 20-25° C.

In a preferred embodiment, the solid LACE molecule is ground up into a fine powder. Preferably, the solid LACE molecule is ground up into a powder with no clumps. In an embodiment, the particle size will be less than 500 microns, in another preferred embodiment, the particle size will be less than 100 microns.

In a preferred embodiment, the pharmaceutical composition is prepared by initial de-aeration of the aqueous solution maintained at room temperature (20-25° C.), then dissolution of the excipients in the solution, followed by adding the solid LACE slowly in parts under vigorous dissolution under nitrogen slow sparging.

in one embodiment, the pharmaceutical composition is stirred vigorously for 4 hours to 24 hours. In a preferred embodiment, the pharmaceutical composition is stirred vigorously from 4 to 8 hours. In another preferred embodiment, the pharmaceutical composition is stirred vigorously for 8 hours.

The pharmaceutical composition prepared by either method can have a shelf-stability of at least 3 months (e.g., 3 months, 6 months, 9 months, 1 year, or more than 1 year),

The pharmaceutical composition can also have favorable profiles of drug related degradant (e.g., total drug related impurities, or amount of a specific drug related impurity) following storage at 5 ° C. for a certain period of time. Analytical tools (e.g., HPLC) for measuring the amount of drug related degradant in a formulation are known.

Suitable biochemically acceptable energy source can be any of those known in the art. For example, the biochemical acceptable energy source can be any of those that can facilitate reduction by participating as an intermediate of energy metabolic pathways, particularly the glucose metabolic pathway. Non-limiting examples of suitable biochemically acceptable energy source include amino acids or derivative thereof (e.g., alanine, glycine, leucine. isoleucine, 2-oxoglutarate, glutamate, and glutamine, etc.), a sugar or metabolites thereof (e,g., glucose, glucose-6-phosphate (G6P)), pyruvate (e.g., ethyl pyruvate), lactose, lactate, or derivatives thereof), a lipid (e.g., a fatty acid or derivatives thereof such as mono-, di-, and tri-glycerides and phospholipids), and others (e.g., NADH). Suitable amount of a biochemically acceptable energy source can be in the range of 0.01% to 5% (e g 0.05%, 0.1%, 0.2%, 0.5%, 1%, 2%, 3%, 4%, 5%, or any ranges based on these specified numeric values) by weight of the composition. In some embodiments, the biochemical energy source is ethyl pyruvate. In some embodiments, the biochemical energy source is alanine. In some embodiments, the amount of ethyl pyruvate or alanine is in the range of 0.05% to 5% (e.g., 0.05%, 0.1%, 0.2%, 0.5%, 1%, 2%, 3%, 4%, 5%, or any ranges based on these specified numeric values) by weight of the composition. In some embodiments, the amount of alanine is 0.5% by weight of the composition. In any of the embodiments described herein, the biochemically acceptable energy source is in an amount that is ophthalmically acceptable.

Suitable preservatives can be any of those known in the art. Non-limiting examples include benzalkonium chloride (BAC), cetrimonium, chlorobutanol, edetate disodium (EDTA), polyquatemium-1 (Polyquad®), polyhexamethylene biguanide (PHMB), stabilized oxychloro complex (PURITE®) sodium perborate, and SofZia®. Suitable amount of a preservative in the pharmaceutical composition can be in the range of 0.005% to 0.1% (e.g,, 0.005, 0.01, 0.02%, 0.05%, 0.1%, or any ranges based on these specified numeric values) by weight of the composition. In some embodiments, the preservative is benzalkonium chloride. In some embodiments, the benzalkoniuin chloride is in the amount of 0.003% to 0.1% (e.g., 0.003, 0.01, 0.02%, 0.05%, 0.1%, or any ranges based on these specified numeric values) by weight of the composition. In some embodiments, the benzalkonium chloride is in the amount of 0.01% by weight of the composition. In any of the embodiments described herein, the preservative is in an amount that is ophthalmically. acceptable. In some embodiments, the pharmaceutical composition is free of a preservative.

Suitable tonicity agents can be any of those known in the art. Non-limiting examples include sodium chloride, potassium chloride, mannitol, dextrose, glycerin, propylene glycol and mixtures thereof. Suitable amount of tonicity agent in the pharmaceutical composition is any amount that can achieve an osmolality of 200-460 mOsni (e.g., 260-360 mOsm, 260-320 mOsm). in some embodiments, the pharmaceutical composition is an isotonic composition. In some embodiments, the amount of a tonicity agent (e.g., sodium chloride) is 0.1% to 5% (e.g., 0.1%, 0.5%, 1%, 2%, 3%, 4%, 5%, or any ranges based on these specified numeric values) by weight of the composition. In any of the embodiments described herein, the tonicity agent is in an amount that is ophthalmically acceptable.

Suitable surfactant can be any of those known in the art, including ionic surfactants and nonionic surfactants. Non-limiting examples of useful nonionic surfactants include polyoxyethylene fatty esters (e.g., polysorbate 80 [poly(oxyethylene)sorbitan monooleate], polysorbate 60 [poly(oxyethylene)sorbitan monostearate], polysorbate 40 [poly(oxyethylene)sorbitan monopalmitate], poly(oxyethylene)sorbitan monolaurate, poly(oxyethylene)sorbitan trioleate, or polysorhate 65 [poly(oxyethylene)sorbitan tristearate]), polyoxyethylene hydrogenated castor oils (e.g., polyoxyethylene hydrogenated castor oil 10, polyoxyethylene hydrogenated castor oil 40, polyoxyethylene hydrogenated castor oil 50, or polyoxyethylene hydrogenated castor oil 60), polyoxyethylene polyoxypropylene glycols (e.g., polyoxyethylene (160) polyoxypropylene (30) glycol [Pluronic F681], polyoxyethylene (42) polyoxypropylene (67) glycol [Pluronic P123], polyoxyethylene (54) polyoxypropylene (39) glycol [Pluronic P85], polyoxyethylene (196) polyoxypropylene (67) glycol [Pluronic F1271], or polyoxyethylene (20) polyoxypropylene (20) glycol [Pluronic L-441]), polyoxyl 40 stearate, sucrose fatty esters, and a combination thereof. In some embodiments, the surfactant is polysorhate 80, Suitable amount of surfactant in the pharmaceutical composition can be in the range of 0.01% to 5% (e.g., 0.05, 0.1, 0.2%, 0.5%, 1%, 2%, 3%, 4%, 5%, or any ranges based on these specified numeric values) by weight of the composition. In some embodiments, the surfactant is polysorbate 80, and the amount of polysorbate 80 is in the range of 0.05% to 5% (e.g., 0.05, 0.1, 0.2%, 0.5%, 1%, 2%, 3%, 4%, 5%, or any ranges based on these specified numeric values) by weight of the composition. In some embodiments, the amount of polysorbate 80 is 0.5% by weight of the composition. In any of the embodiments described herein, the surfactant is in an amount that is ophthalmically. acceptable. :However, in sonic embodiments, the pharmaceutical composition is free of a surfactant.

Suitable viscosity modifying agent can be any of those known in the alt. Non-limiting examples include carbopol gels, cellulosic agents (e.g., hydroxypropyl methylcellulose), polycarbophil, polyvinyl alcohol, dextran, gelatin glycerin, polyethylene glycol, poloxamer 407, polyvinyl alcohol and polyvinyl pyrrolidone and mixtures thereof. Suitable amount of viscosity modifying agent can be in the range of 0.1% to 5% (e.g., 0.1%, 0.5%, 1%, 2%, 3%, 4%, 5%, or any ranges based on these specified numeric values) by weight of the composition. In any of the embodiments described herein, the viscosity modifying agent is in an amount that is ophthalmically acceptable. In some embodiments, the pharmaceutical composition is free of a viscosity modifying agent (e.g., a polymeric viscosity modifying agent such as hydroxypropyl methylcellulose).

In some embodiments, the pharmaceutical composition is characterized by one or more of the following:
(a) having a concentration of the lipoic acid choline ester salt from 0.1% to 10% (e.g., 0.1%, 1.0%, 1.5%, 3%, 4%, 5%, or any ranges between the specified numeric values) by weight of the composition;
(b) having a concentration of a preservative (e.g., benzalkonium chloride) of 0.003% to 0,1% (e.g., 0.01%) by weight of the composition;
(c) having a biochemical energy source (e.g., alanine) of 0.1% to 5% (e.g., 0.5%) by weight of the composition; and
(d) having a concentration of glycerol of 0.5% to 5% (e.g., 2.7%) by weight of the composition.
e) having a concentration of hydroxypropyl beta cyclodextrin of 1% to 20% by^{,} weight of the composition.
f) having a concentration of hydroxypropyl methyl cellulose (UPMC) of 0.1-0.5% by weight of the composition.

In some embodiments, the pharmaceutical composition consists essentially of 1-3% by weight of glycerin, 0.5% by weight of alanine, 0.005-0.01% by weight of benzalkonium chloride, 1-3% by weight of lipoic acid choline ester, and water, wherein the pH of the pharmaceutical composition is 4.3 to 4.7.

In some embodiments, the pharmaceutical composition consists essentially of 1-3% by weight of glycerin, 0.5% by weight of alanine, 1-30% hydroxypropyl beta cyclodextrin, 0.005-0.01% by weight of benzalkonium chloride, 1-3% by weight of a pharmaceutical salt of lipoic acid choline ester, and water, wherein the pH of the pharmaceutical composition is 4.3 to 4.7.

In another embodiment, the pharmaceutical salt form of lipoic acid choline ester is a chloride.

In another embodiment, the pharmaceutical salt form of lipoic acid choline ester is an iodide.

In another embodiment, the pharmaceutical salt form of lipoic acid choline ester is among the group, but not limited to chloride, bromide, iodide, mesylate, phosphate, tosylate, stearate. methanesulfonate.

In another embodiment, the viscosity enhancing agent is methyl cellulose, hydroxypropyl methyl cellulose, polyvinyl alcohol, polyvinyl pyrrolidone.

In a preferred embodiment, the preferred viscosity enhancing agent is hydroxypropyl methyl cellulose in concentrations 0.1-0.5%.

In another embodiment, an antioxidant is added to stabilize LACE.

Suitable anti-oxidants can be ascorbates, glutathione, histidine, methionine, cysteine.

In another embodiment, the pIl of the composition is between 4 and 5.

In one embodiment, the ophthalmic composition is dosed to each eye of the subject once daily, twice daily, thrice daily and four times daily.

In some embodiments, the invention also provides a system for storing a pharmaceutical composition comprising an active ingredient in an aqueous solution, wherein the active ingredient (e.g., lipoic acid choline ester or derivatives thereof) is susceptible to hydrolysis in the aqueous solution. In a preferred embodiment, the pharmaceutical composition is stored in a LDPE ophthalmic eye-dropper bottle, overlaid with nitrogen during the filling process, capped, then packed in a secondary mylar, gas-impermeable pouch containing an oxygen absorbent.

In another embodiment, the eye-dropper bottle or unit is polyethylene terephthalate (PET). In another embodiment, the eye-dropper bottle is constructed of a material that has low gas permeability.

In another embodiment, the eye-dropper bottle or unit is a glass ophthalmic bottle with a polypropylene dropper tip for dispensation into the eye.

In other embodiment, eyedropper bottle can be constructed of any material that has a low gas permeability. In another embodiment, the eye-dropper bottle can he unit dose, filled by blow fill seal techniques.

In one embodiment, the pharmaceutical composition is stored at 2-5° C. for a period of 3 months to 2 years.

### Methods of Treatment

The pharmaceutical compositions comprising lipoic acid choline ester or derivatives thereof (e.g., as described herein) can be employed in a method for treating or preventing a disease or disorder associated with oxidative damage. Diseases or disorders associated with oxidative damage are known.

In some embodiments, the invention provides a method of treating an ocular disease in a subject in need thereof, comprising administering to an eye of the subject a therapeutically effective amount of any of the pharmaceutical compositions described herein.

In some embodiments, the ocular diseases are presbyopia, dry eye, cataract, macular degeneration (including age-related macular degeneration), retinopathies (including diabetic retinopathy), glaucoma, or ocular inflammations. In some embodiments, the ocular disease is presbyopia.

Suitable amount of pharmaceutical compositions for the methods of treating or preventing an ocular disease herein can he any therapeutically effective amount. In some embodiments, the method comprises administering to the eye of the subject an amount of the phannaceutical composition effective to increase the accommodative amplitude of ⁻the lens by at least 0,1 diopters (D) (e.g., 0.1, 0.2, 0.5, 1, 1.2, 1.5, 1.8, 2, 2.5, 3, or 5 diopters). In some embodiments, the method comprises administering to the eye of the subject 1-5 drops (about 40 uL per drop) of the pharmaceutical composition. In some embodiments, the eye of the subject is treated with the pharmaceutical composition 1, 2, 3, 4, 5, or more than 5 times a day, each time with 1-5 drops (about 40 μL per drop). In some embodiments, the lens or eye of the subject is treated with the pharmaceutical composition 1, 2, 3, 4, 5, or more than 5 drops each time. In some embodiments, the eye of the subject is treated with the pharmaceutical composition herein twice or three times per day, each time with 1 or 2 drops (about 40 uL per drop).

The methods include preventative methods that can be performed on patients of any age. The methods also include therapeutic methods that can be performed on patients of any age, particularly patients that are between 20-75 years of age.

The following examples are illustrative and do not limit the scope of the claimed embodiments.

### EXAMPLES

### Example 1

### Chemical Structure and General Properties of Lipoic Acid Choline Ester Chloride (LACE)

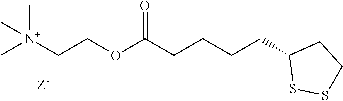 [SMILES: C[N+](C)(C)CCOC(=O)CCCC[C@@H]1CCSS1]

TABLE 1
General Properties of Lipoic Acid Choline Ester (LACE)

| Appearance: | |
|---|---|
| Solubility Profile: | >50 mg/mL in water |
| | >4 mg/mL in acetonitrile |
| Solution pH | 7-7.5 |
| Log P | <2 |
| Specific rotation: | 70.3° |
| Optical rotation: | 0.338 at 25° C. at 0.005 |
| | g/mL in acetonitrile |
| Spectral properties: | UV Aₘₐₓ 333 nm |
| Hygroscopicity: | Highly hygroscopic |
| Crystallinity: | Sharp crystalline melt transition |
| | not observed, mostly amorphous |
| Polymorphs: | Not known at this time |
| Particle size: | D₅₀: 100-200 mm |
| Melting/boiling range: | Thermal transition observed |
| | at 230-235° C. |

### Example 2

### Kinetics of Micellar Species Correlated to the Duration of Mixing of LACE Chloride Process Solutions

The experiments described in this section demonstrate that the LACE Chloride micellar species stabilize and diminish over extended mixing times at 25° C. The results demonstrated that the reversible nature of these species characteristic of self-assembled systems such as micelles and micellar aggregates.

Micellar species formed by spontaneous self-assembly of molecules are driven by the total free energy of the equilibrated system. The experiment demonstrated the kinetics of achievement of that equilibrated state with longer durations of mixing.

### Objectives:

Establish process-time bracket by establishing if growing micellar species has stabilized.
Establish “holding time”

### Procedure:

Two 200-g batches of 1.5% LACE Chloride were prepared at 25° C. after vehicle was deoxygenated with bubbled nitrogen. Nitrogen was continually bubbled during dissolution of LACE.
One batch was prepared using GMP Batch #2 (G2-14LAC) as is, with significant clumps, the other was prepared using a sample of G2-14LAC that had been ground into a fine powder using a mortar and pestle.
After dissolution of the LACE and pH adjustment, the batches were stirred. for 24 hours with a constant nitrogen overlay, maintaining dissolved oxygen at ˜1.6 ppm (vs. 8.2 ppm saturated solubility). At time-points of 1 h, 3 h, 4 h, 6 h, 8 h, 9 h, and 24 h, about 5-15 mL was removed by syringe and sterile-filtered into eye-dropper bottles (5 mL/bottle), without nitrogen overlay in the bottle (apparatus was in use for the bulk batches).
Samples of all time-points were diluted to 10 mg/g, and then injected for RP-HPLC analysis with ELK.) detection within 30 minutes of removal from the bulk solution.
After the 24-hour time-point, bulk solutions were sterile-filtered, and each divided into two ˜50 mL portions—one held at 5° C., the other 50-mL portion held at 25° C. All portions were overlaid with nitrogen blown into the vessel.
At the end of the additional 24-hour hold time, each portion was filled into eye-dropper bottles with a nitrogen overlay in the bottle.

### Observations on Dissolution

The clumped portion of G2-14LAC was added to formulation KW-LACE-01-86-1 over about 5 minutes, and some of the clumps required another 20 minutes to dissolve.
The powdered G2-14LAC was added to formulation KW-LACE-01-86-2 over about 15 minutes, because each spatula-full aggregated into a thin raft of material floating on the surface, which did not disperse immediately. Therefore another portion was not added until previous portions were drawn into the vortex. Estimated time for any one portion to dissolve was about 10 minutes, and the whole process took approximately 25 minutes.

### Results

A peak at RI=8 minutes (correlated with the micellar species) was evident in both formulations from the first time-point taken.
There were no consistent differences between the two batches in the % Area of the 8 min. peak (micellar species), though the 2^{nd} batch, made with powdered LACE chloride, had higher levels of the micellar species at some time-points.
The %Area of the 8 min. peak was significantly reduced at 24 hours, as shown in the table below.
Final pH was 4.54, for both batches.

TABLE 2
Kinetics of the Formation and De-Agglomeration of LACE
Micellar Species with Extensive Mixing of LACE Chloride

| | Percent Area of RT = 8 min. broad peak | |
|---|---|---|
| | (LACE Micellar Species) | |
| | Total area of LACE and related peaks | |
| | (Results are for 1^{st} injection from HPLC vial, unless noted) | |
| | KW-LACE-01-86-1 | KW-LACE-01-86-2 |
| | 1.5% LACE, made from | 1.5% LACE, made from |
| | G2-14LAC with clump | G2-14LAC ground up |
| Mixing Time | % micellar species | % micellar species |
| (Hours) | at 8 minutes | at 8 minutes |
| 1 hour | 0.30% | 0.63% |
| 2 hours | 0.51% | 0.56% |
| 3 hours | 0.56% | 0.67% |
| 4 hours | 0.50% | 0.44% |
| 6 hours | 0.64% | 0.70% |
| 8 hours | 0.47% | 0.44% |
| 9 hours | 0.42% | 0.44% |
| 24 hours | 0.07% | 0.17% |
| 48 h (5° | Not detected | 0.09% |
| C. hold) | | |
| 48 h (25° | Not detected | Not detected |
| C. hold) | | |

The results demonstrate that LACE Chloride micellar species at 8.1 minute are minimized with extended mixing times. The peak at 8.1 minutes is diminished dramatically with longer mixing times.

Each of the solutions was also measured for degradants of lipoic acid choline ester. As mentioned earlier, the degradation mechanism of LACE is oxidative and hydrolytic, resulting in oxidized and hydrolyzed species.

TABLE 3
Impurity (Related Substances) Analysis of EV06 Ophthalmic Solution as a Function of Mixing

| | KW-LACE-01-86-1:G2-14LAC with clumps | | | | | | |
|---|---|---|---|---|---|---|---|
| | | TimePoint [symbol] | | | | | |
| | | 1 hour | | 3 hours | | 4 hours | |
| Related | Specification | RRT 0.50 | 0.28% | RRT 0.50 | 0.37% | RRT 0.50 | 0.38% |
| | | RRT 0.54 | 0.14% | RRT 0.54 | 0.16% | RRT 0.54 | 0.16% |
| | Individual | RRT 0.57 | 0.16% | RRT 0.57 | 0.18% | RRT 0.57 | 0 18% |
| Compounds | Report 2: 0.1% | Lipoic Acid | 0.02% | Lipoic Acid | 0.03% | Lipoic Acid | 0.03% |
| | (% Area) | RRT 1.61 | 0.17% | RRT 1.61 | 0.17% | RRT 1.61 | 0.16% |
| | Total: Report | Total | 0.77% | Total | 0.91% | Total | 0.91% |
| | KW-LACE-01-86-1:G2-14LAC with clumps | | | | | | |
| | | TimePoint [symbol] | | | | | |
| | | 6 hours | | 8 hours | | 9 hours | |
| Related | Specification | RRT 0.50 | 0.43% | RRT 0.50 | 0.47% | RRT 0.50 | 0.49% |
| | | RRT 0.54 | 0.17% | RRT 0.54 | 0.17% | RRT 0.54 | 0.18% |
| | Individual | RRT 0.57 | 0.19% | RRT 0.57 | 0.20% | RRT 0.57 | 0.20% |
| Compounds | Report 2: 0.1% | Lipoic Acid | 0.03% | Lipoic Acid | 0.03% | Lipoic Acid | 0.03% |
| | (% Area) | RRT 1.61 | 0.16% | RRT 1.61 | 0.16% | RRT 1.61 | 0.16% |
| | Total: Report | Total | 0.98% | Total | 1.02% | Total | 1.05% |
| | KW-LACE-01-86-1:G2-14LAC with clumps | | | | | | |
| | | TimePoint [symbol] | | | | | |
| | | | | 48 hours | | 48 hours | |
| | | | | 5° C. hold | | 25° C. hold | |
| | | 24 hours | | after first 24 h | | after first 24 h | |
| Related | Specification | RRT 0.50 | 0.65% | RRT 0.50 | 0.68% | RRT 0.50 | 0.89% |
| | | RRT 0.54 | 0.20% | RRT 0.54 | 0.17% | RRT 0.54 | 0.21% |
| | Individual: | RRT 0.57 | 0.25% | RRT 0.57 | 0.20% | RRT 0.57 | 0.27% |
| Compounds | Report 2: 0.1% | Lipoic Acid | 0.04% | Lipoic Acid | 0.05% | Lipoic Acid | 0.06% |
| | (% Area) | RRT 1.61 | 0.16% | RRT 1.61 | 0.17% | RRT 1.61 | 0.20% |
| | Total: Report | Total | 1.31% | Total | 1.27% | Total | 1.61% |

The data shows that the degradation products of LACE rise with extended mixing time. Thus, final process conditions for the compounding of EV06 Ophthalmic Solution involved a maximum of 8 hours to achieve a non-irritating solution with minimized degradants.

A similar mixing experiment performed with LACE Iodide did not result in a solution that had minimal aggregation. In fact, in the case of LACE iodide, the aggregated species were as high as 39% of the API at the end of 8 hours of mixing.

### Example 3

### Correlation of Mixing Temperature with Presence of Micellar LACE Chloride Species

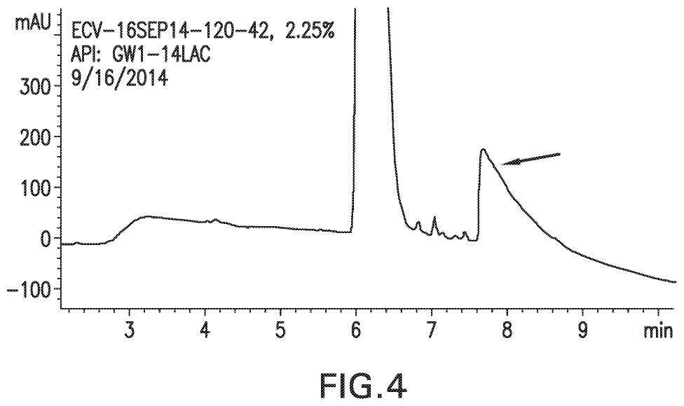

The data shown in FIG. 4 is of a solution of LACE Chloride formulated under argon and refrigerated conditions. The solution was extremely irritating to the ocular surface. The percent micellar species was 8-10% of the main LACE API Peak (micellar species denoted by arrow, at retention time 7.9-8.1 minutes), a concentration that is normally not observed in solutions mixed at room temperature.

### Example 4

### Correlation of the Clumps to the Formation of Micellar LACE Species

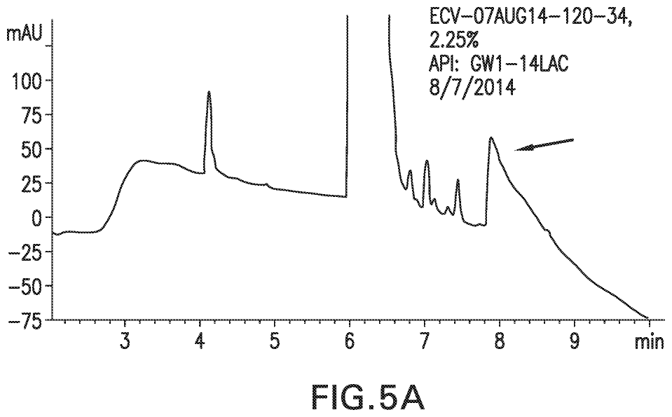

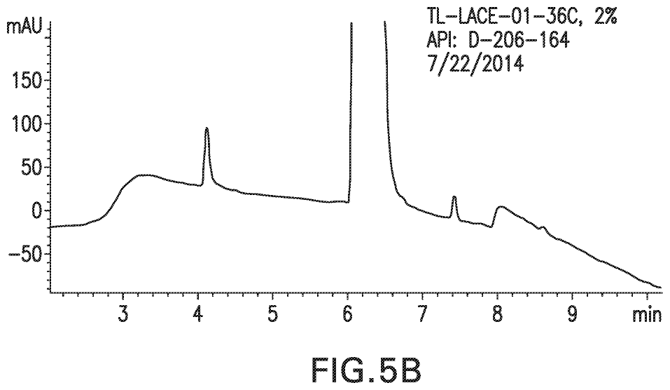

FIG. 5A is a RP-HPLC chromatogram of EV06 Ophthalmic Solution prepared from a LACE Chloride hatch that had solid “clumps”. The solution prepared from this lot of API (active pharmaceutical ingredient, solid LACE drug substance) showed a higher percentage (10-15%) of the micellar LACE species (shown with an arrow) than solutions prepared from a lot of API that was powdery (FIG. 5B).

Thus, while both solutions looked completely dissolved, the solution formulated from non-clumped API had a lower concentration of micellar LACE species (see FIG. 5B). When correlated to ocular irritation, the solution shown in FIG. 5A had higher scores for irritation in a rabbit model. This led to incorporation of de-clumping procedures to render powdered material, prior to compounding.

### Example 5

### Compatibility Studies of Excipients with LACE

### Summary

The purpose of these experiments was to tease out possible destabilizing variables in the formulation, through systematic variations in formulation composition and micro-environment (such as pH). Lipoic acid, and any derivatives of lipoic acid would be subject to degradation and polymerization in heat, light and oxygen, leading to opening of the dithiolane ring. Thus, presence of excipients that can induce oxidative free radical scission could be destabilizing factors. The formulation grids 1 and 2, systematically investigated the effect of excipients already present in the formulation as possible destabilizing factors.

The formulation composition for LACE in these experiments contains the drug substance, alanine, glycerin, benzalkonium chloride in purified water, in 1N sodium hydroxide, or 1N hydrochloric acid added to achieve a pH between 4.4-4.6 and an osmolality of 290-300 mOsm/kg. The experiments described in this document were compatibility studies to identify excipients that could stabilize LACE ophthalmic solutions.

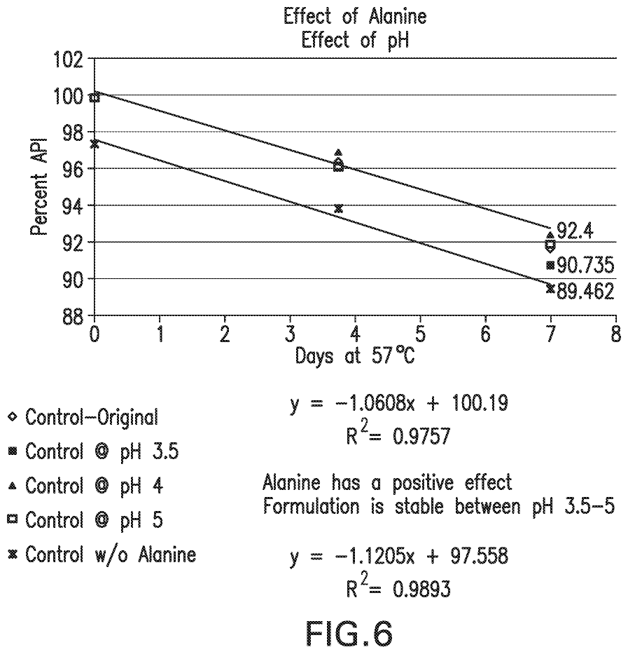

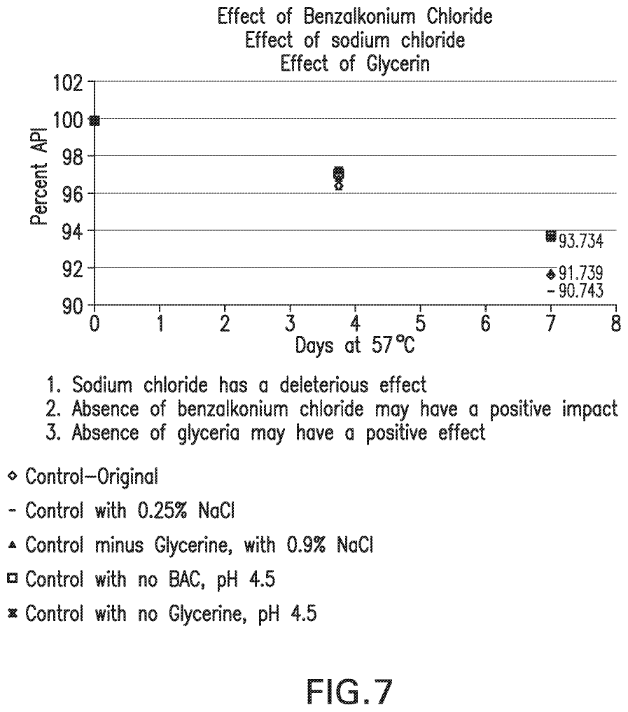

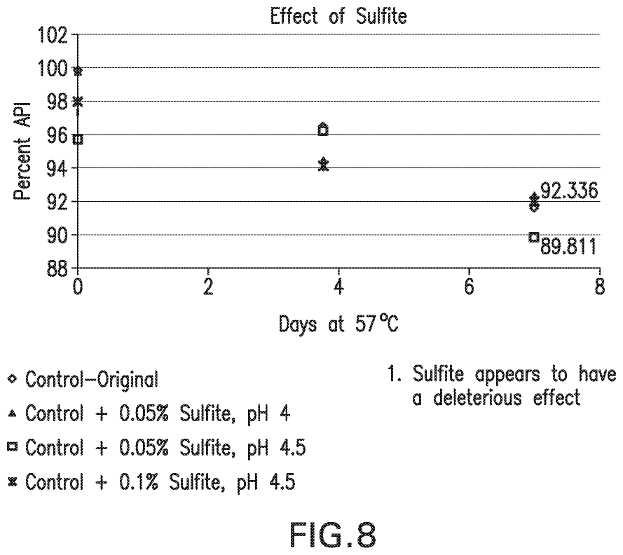

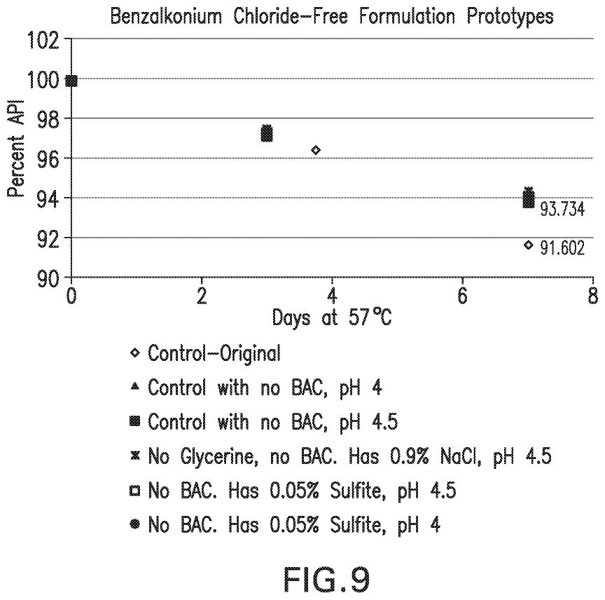

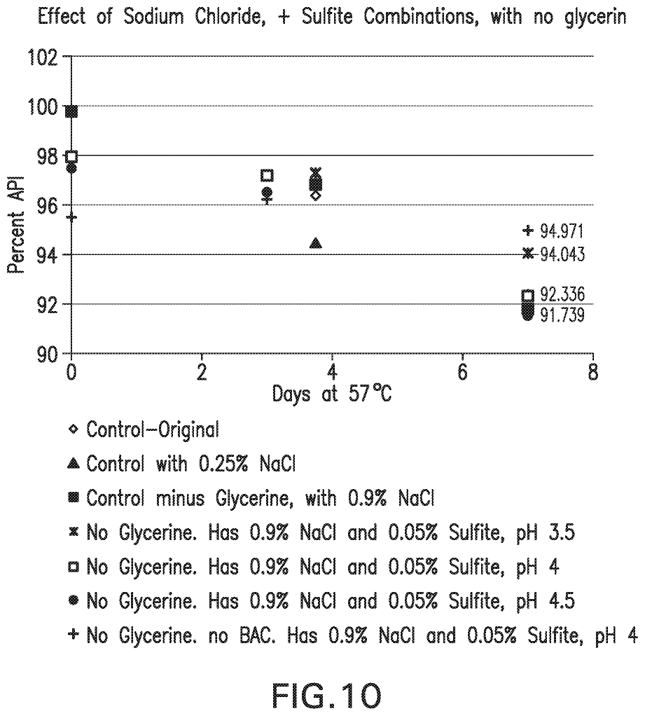

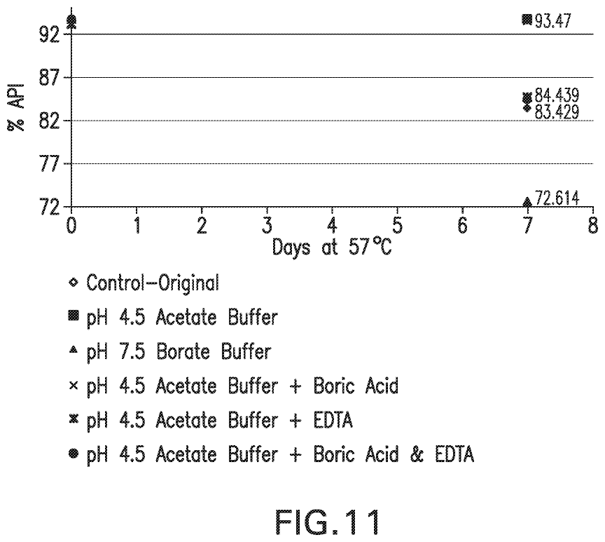

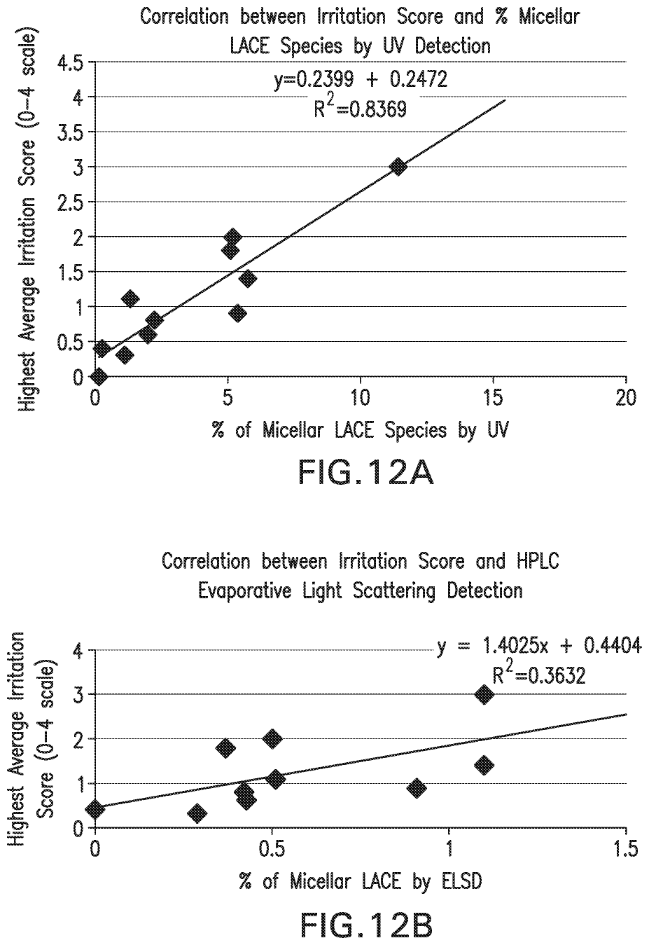

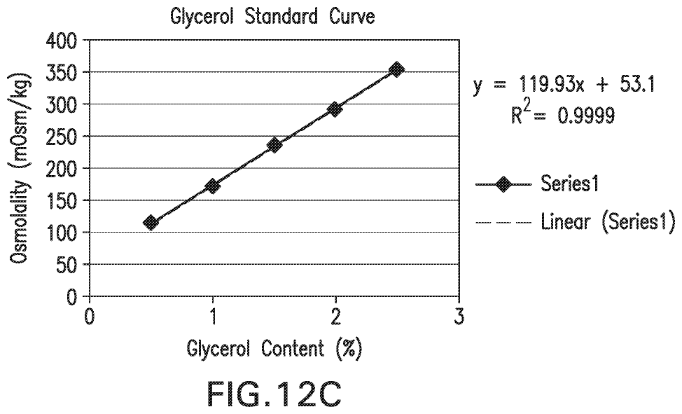

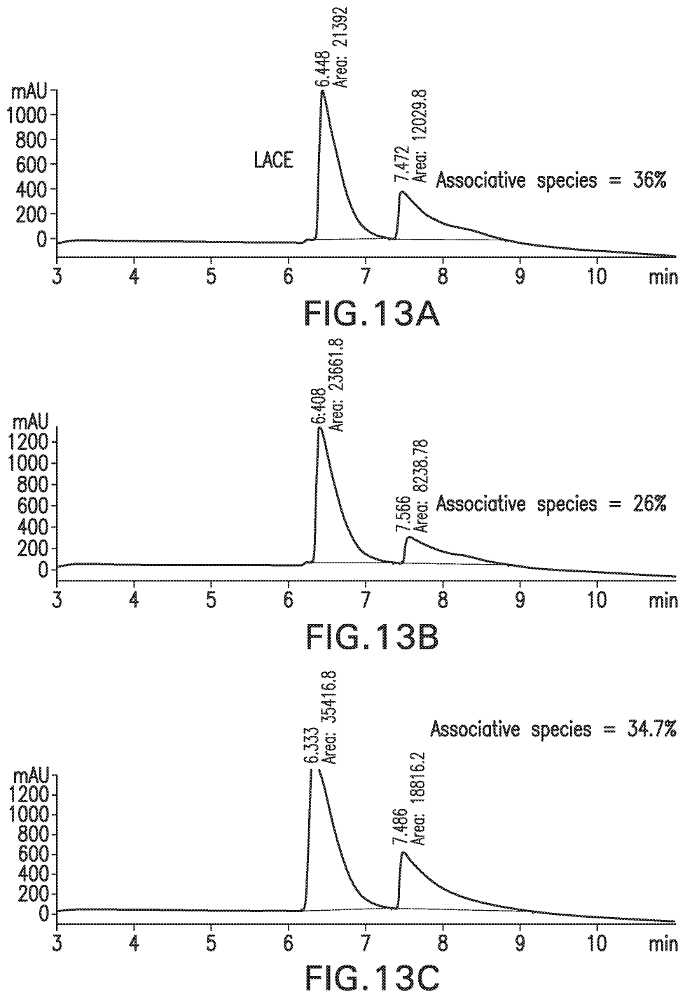

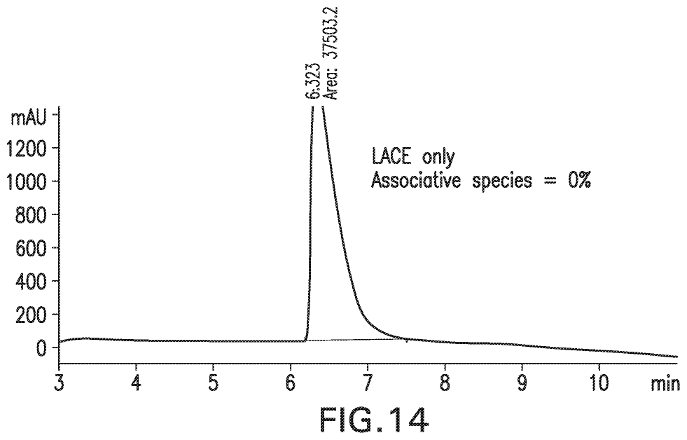

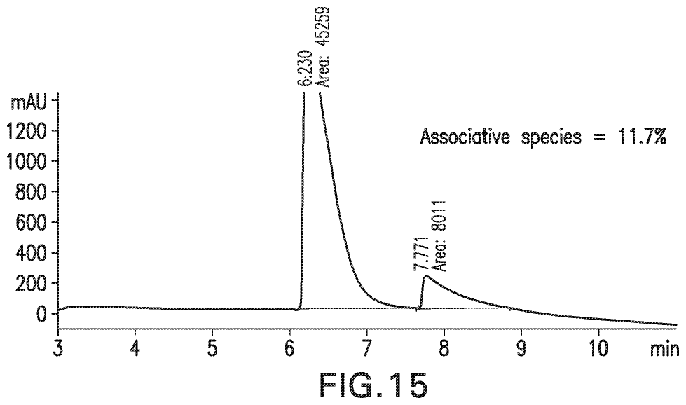

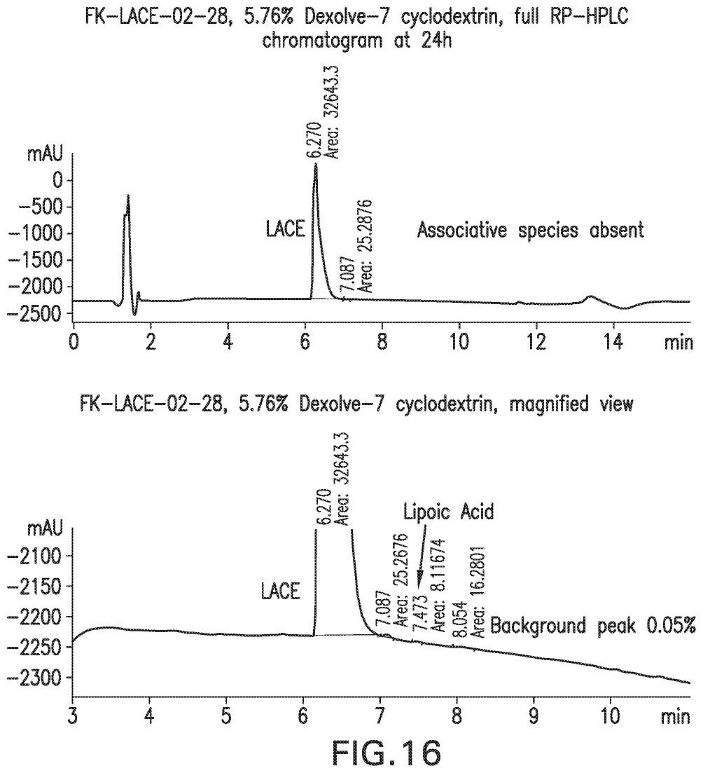

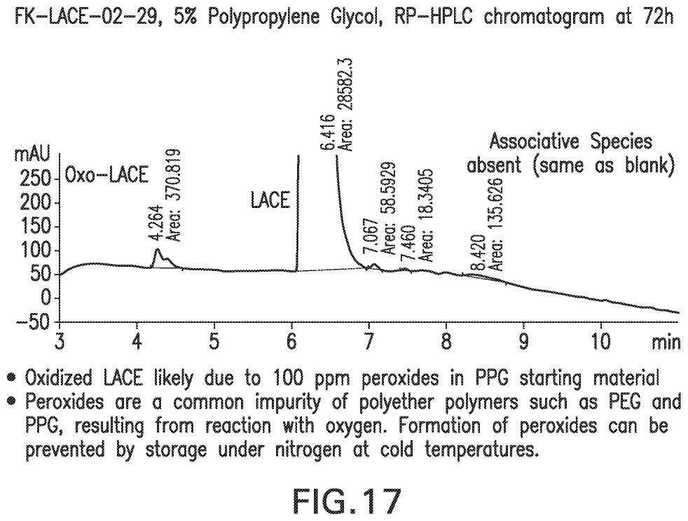

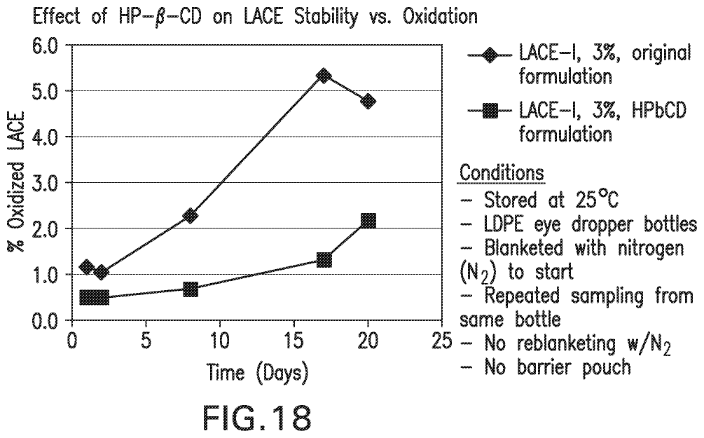

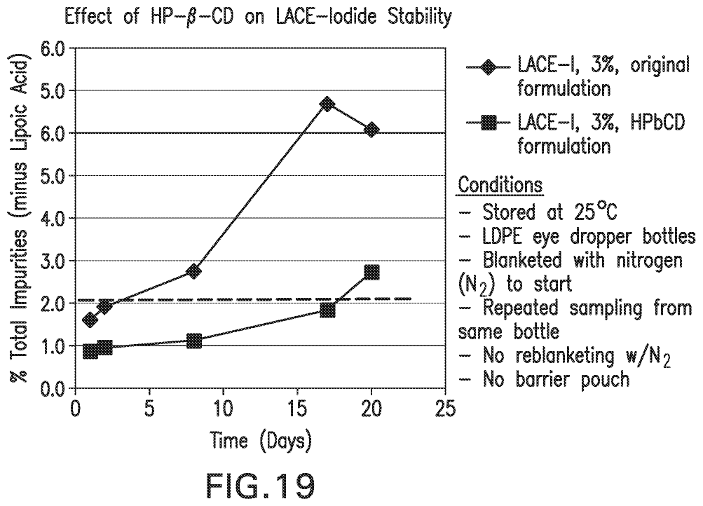

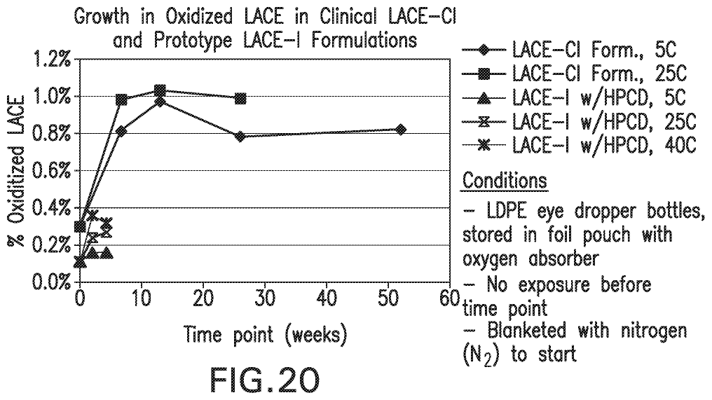

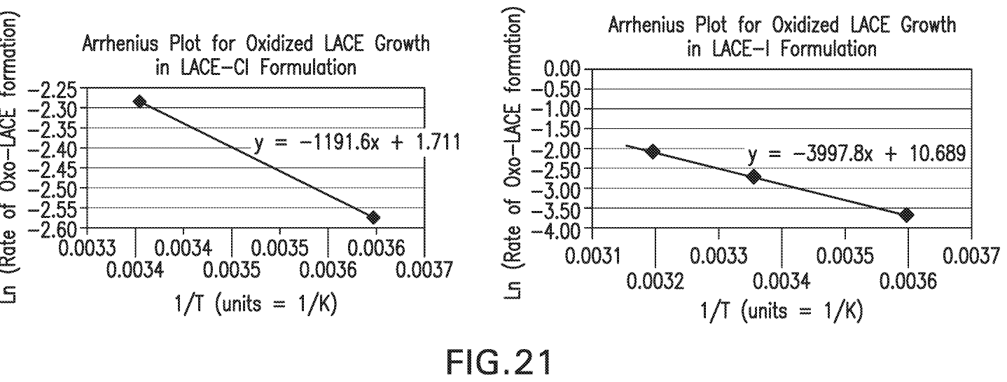

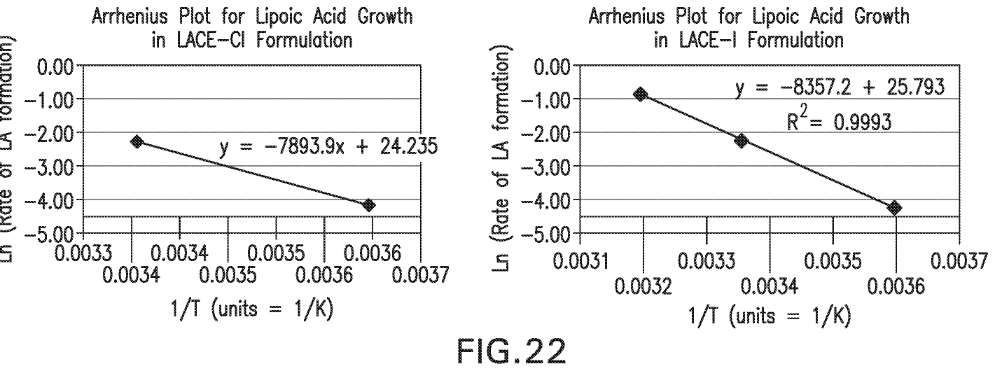

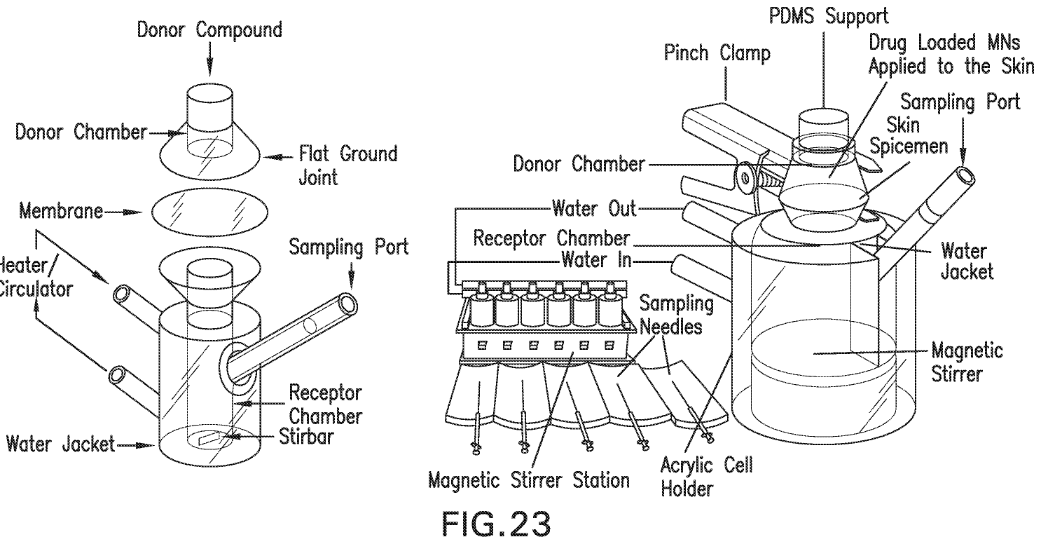

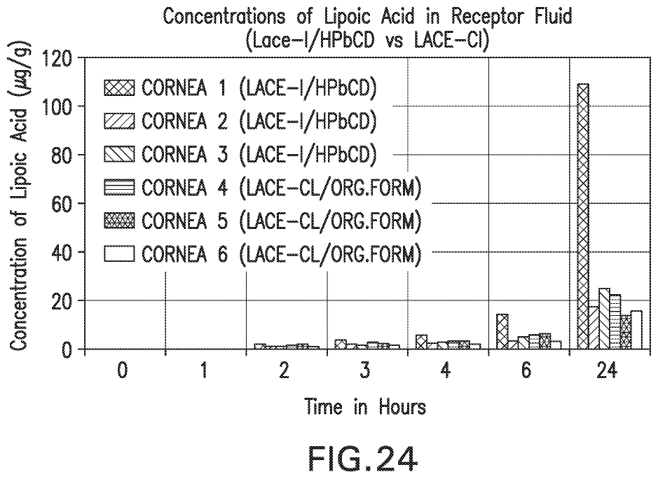

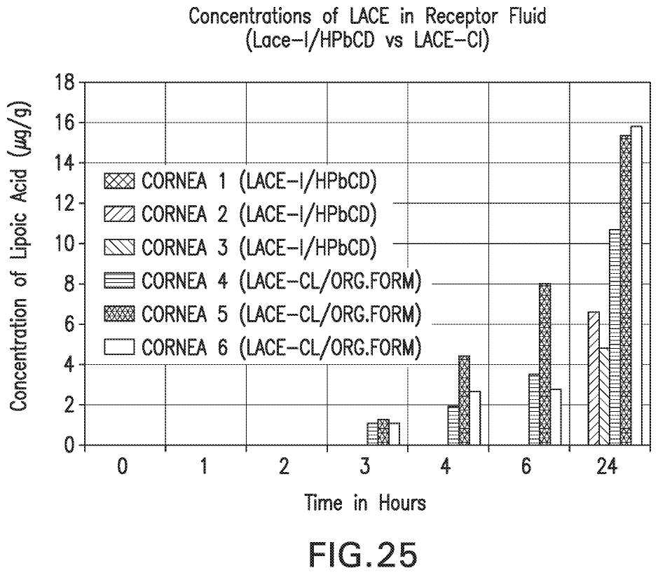

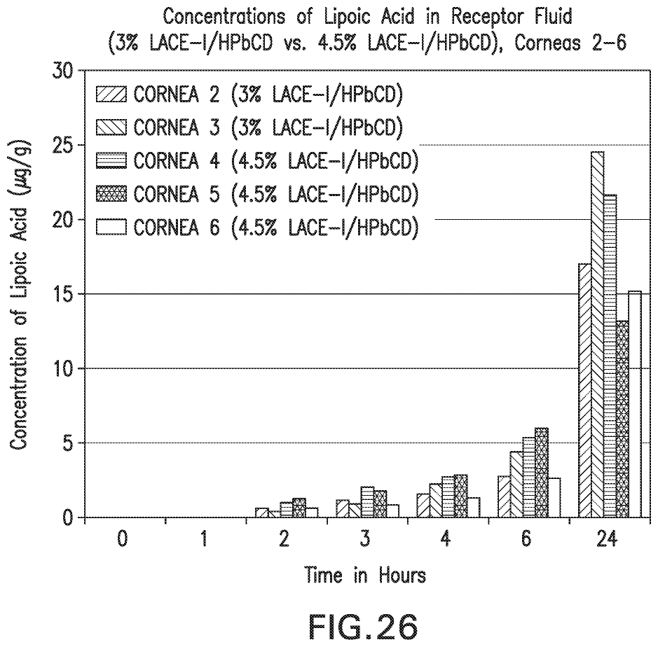

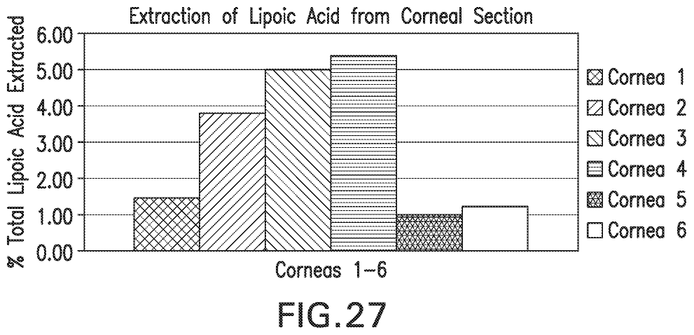

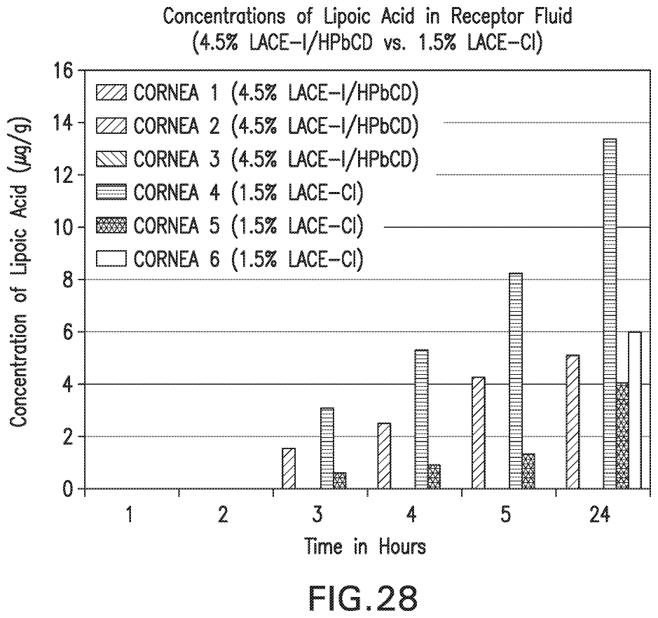

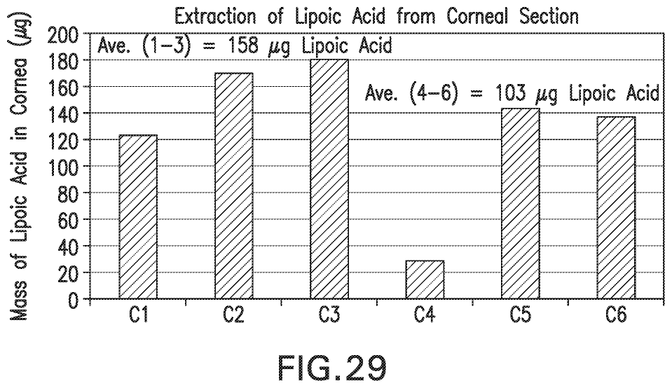

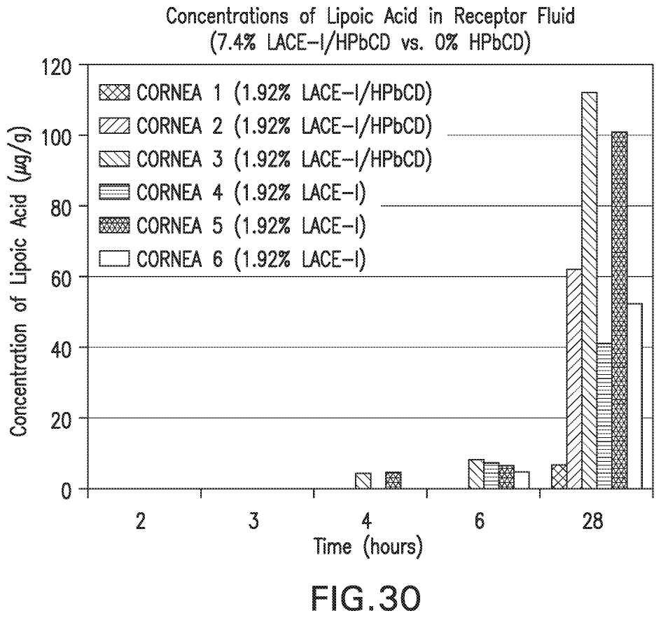

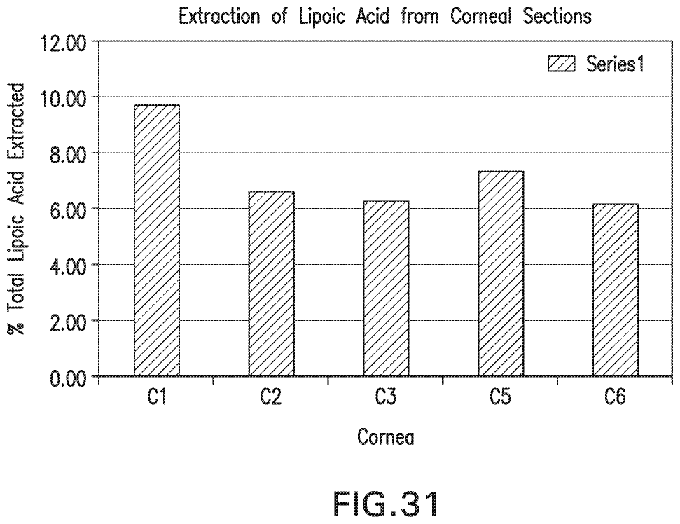

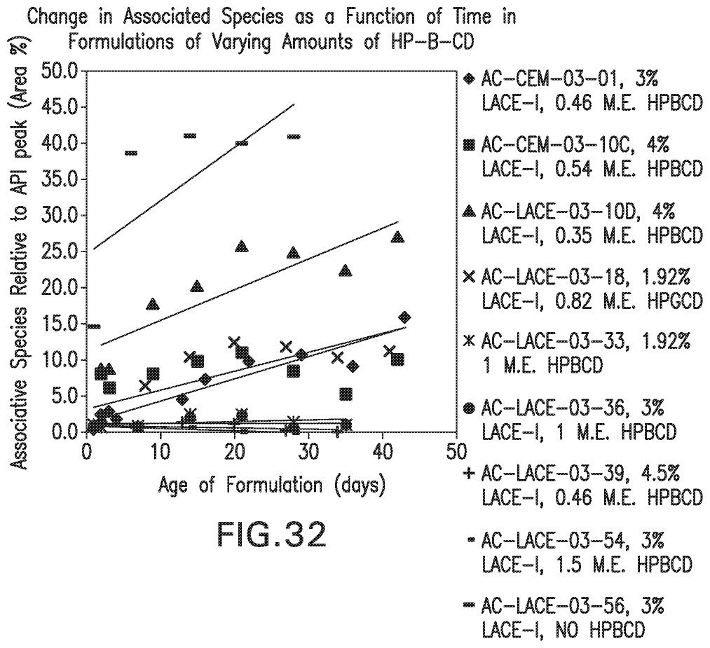

Formulation Grid#1 tested the following variables given in (a)-(e). The formulations were prepared in a nitrogen-flushed glove box and sterile filtered. All formulations were tested under accelerated conditions at 57° C. and tested by HPLC for assay and impurities at T=0, 3.5 days and 7 days. A total of 19 formulations were tested in Grid#1.
(a) Effect of pH: Formulations were prepared at pH 3.5, 4 and 5, and compared with the control formulation at pH 4.5, As shown in FIG. 6, the rate of degradation of LACE was equivalent at all pH levels in the range 3.5-5.
(b) Effect of Alanine: The role of alanine in the formulation was deduced, by comparison of rates of degradation with the original formulation (control). As shown in FIG. 6, the absence of alanine appeared to accelerate the rate of degradation of LACE. Thus, Alanine is a critical excipient in EV06 Ophthalmic formulations.
(c) Effect of Benzalkonium Chloride and Glycerin: it was hypothesized that peroxides contained in glycerin can catalyze oxidation similarly, it was hypothesized that BAK could destabilize the drug substance, due to free radical scission and subsequent oxidation. As seen in FIG. 7, the benzalkonium chloride-free formulation was substantially more stable than the control. The glycerin-free prototype was also more stable than the control. Additionally, sodium chloride added into the formulation (instead of glycerol, to adjust osmolality) appeared to have a destabilizing effect (also shown in FIG. 7) In another experiment with various combinations of glycerin, sodium chloride, sulfite and pH with all variations being benzalkonium chloride-free, it was remarkable that all of the benzalkonium chloride-free formulations were more stable than the control (FIG. 5). The experiments in FIGS. 7 and 9 demonstrate that eliminating benzalkonium chloride in LACE may be a method to stabilize the formulation. For EV06 Ophthalmic compositions, minimizing benzalkonium chloride content to 50 ppm may have a major stabilizing factor. Sodium chloride demonstrated a destabilizing effixt, thus glycerol was deemed more suitable as a tonicity agent in final EV06 compositions.
(d) Effect of Sulfite: Various experiments were performed with sulfite (FIG. 8), with combinations of various levels of sulfite. Sulfite was added to the formulation as an anti-oxidant (FIG. 8), at various pH levels (4, 4.5) and concentrations. The presence of sulfite did not appear to substantially improve the stability the LACE. It was not clear if a deleterious effect was present, since 0.1% sulfite in the formulation was equivalent to the control.
(e) Effect of glycerin: The effect of glycerin was investigated in various formulation combinations, by the systematic elimination of glycerin. As shown in FIGS. 7 and 10, ⁻the glycerin-free combinations appeared to be more stable than control. However, due to the high destabilizing effect of sodium chloride, glycerin was selected as the critical excipient for tonicity adjustment.
(f) Effect of Buffer: Various buffered compositions were tested. Acetate buffer and acetate +boric acid appeared to stabilize the formulation.

### Experimentals

a) HPLC Method Setup: The HPLC assay consisted of a 50 minute mobile phase gradient made up of (A) 0.05M sodium phosphate monobasic, 0.005M 1-heptane sulfonic acid sodium salt, 0.2% v/v triethylamiue, adjusted to pH 4.5 with phosphoric acid; and (B) acetonitrile. The analytical column used is a YMC Pack ODS AO (4.6×250 mm, 5 μm, 120 Å), P/N AQ125052546WT; the analytical detection wavelength is 225 nm.

### b) Formulations

Formulations were prepared with extensive care to ensure that the LACE API was not exposed to oxygen or heat. The API was aliquotted into clean glass vials under an inert N₂ atmosphere inside of glove bag, and stored wrapped in tinfoil in a −20° C. freezer until use. The formulations were prepared with high purity excipients, and sterile glassware. All excipients were pre-prepared in stock solutions and were mixed together before the addition of API & final pH adjustments. The formulations are tabulated in Appendix A.

### II. RESULTS AND DISCUSSION

FIG. 6 is a plot of %API versus time at 57° C. (over T=0, 3.5 days and 7 days), systematically comparing formulations that were prepared at pH 3.5. 4, 4.5 (original), 5 and control without alanine. Even at T=0, the formulation without alanine had degraded considerably in API content. As seen in FIG. 6, the formulations were equivalent under these conditions at pH 3.5-5.

FIG. 7 is a plot of formulations comparing the following variables: (a) Control (original) versus Control+025% sodium chloride, Control 0.25% sodium chloride without glycerin, (h) Control (original) versus control without henzalkonium chloride, (c) Control (original) versus original formulation without glycerin.

As seen in FIG. 7, addition of sodium chloride to the original formulation did not stabilize the formulation.

FIG. 8 shows the effect of sulfite on LACE stability at 57° C. Sulfite-containing formulations were prepared at concentrations 0.05% sulfite and 0.1% sulfite at 4 and 4.5. Addition of sulfite did not stabilize the original formulation.

FIG. 9 further explores the potentially stabilizing effect of eliminating benzalkonium chloride. Formulation variations without henzalkonium chloride were superior to the control original formulation (pH 4.5). Formulation variations were BAC-free compositions at pHs 4, 4.5, no glycerin/no BAC+0.9% sodium chloride, no BAC+0.05% sulfite at pHs 4 and 4.5.

FIGS. 7 and 10 compare the effect of glycerin, in various compositions, as a function of pH, sulfite and sodium chloride. The no-glycerin, no-BAC formulation in the presence of sodium chloride and sulfite and the no-glycerin with BAC formulation were superior to the original formulation.

FIG. 11 explored the use of various buffered compositions on LACE stability. The original formulation (pH 4.5) was compared with acetate buffer compositions and borate at pH 7.5. Sodium edetate added as an anti-oxidant did not stabilize the formulation. Acetate buffer and acetate buffer plus boric acid appeared to be superior to the control formulation.

To summarize, elimination of benzalkonium chloride appeared to enhance stability consistently. Elimination of glycerol may be a positive step as well. Glycerol is known to have residual presence of formaldehyde which occasionally leads to degradation of API. Interestingly, addition of edetate or sulfite did not have a positive effect. Another anti-oxidant such as sodium ascorbate may have a positive effect.

TABLE 4
Compatibility Experiment Formulations
Formulation # 1

| | | grams | grams | Act. |
|---|---|---|---|---|
| Ingredient | Desired % | needed | used | w/w % |
| Lipoic Acid Choline | 1 | 0.2 | 0.1849 | 0.9253 |
| Ester (LACE) | | | | |
| Alanine | 0.5 | 0.1 | 2.0254 | 0.5068 |
| Glycerine | 0.1 | 0.02 | 0.401 | 0.0996 |
| Benzalkonium | 0.01 | 0.002 | 0.0378 | 0.0094 |
| Chloride | | | | |
| Ultrapure Water | 75 | 15 | 12.338 | 61.7409 |
| (1st addition) | | | | |
| 1N NaOH | Used to adjust to desired pH | | 0.0815 | 0.0143 |
| 1N HCl | before final water addition | | | |
| Ultrapure Water | qs | qs | 4.9149 | 24.5948 |
| (2nd addition) | | | | |
| Total | 100 | 20 | 19.9835 | |
| Desired pH | Final pH | mOsm/L | | |
| 4.5 | 4.55 | | | |
| | Desired % | grams | grams | Act. |
| Ingredient | w/w | needed | used | w/w % |

Formulation # 2

| Lipoic Acid Choline | 1 | 0.0 | 0.1977 | 0.9923 |
|---|---|---|---|---|
| Ester | | | | |
| Alanine | 0 | 0.02 | 0 | 0.0000 |
| Glycerine | 0.1 | 0.002 | 0.4009 | 0.0998 |
| Benzalkonium | 0.01 | 0 | 0.0386 | 0.0097 |
| Chloride | | | | |
| Ultrapure Water | 75 | 15 | 14.4079 | 72.3176 |
| (1st addition) | | | | |
| 1N NaOH | Used to adjust to desired pH | | 0.1185 | 0.0209 |
| 1N HCl | before final water addition | | | |
| Ultrapure Water | qs | qs | 4.7595 | 23.8894 |
| (2nd addition) | | | | |
| Total | 100 | 20 | 19.9231 | |
| Desired pH | Final pH | mOsm/L | | |
| 4.5 | 4.48 | | | |

Formulation # 3

| Lipoic Acid Choline | 1 | 0.2 | 0.1993 | 0.9935 |
|---|---|---|---|---|
| Ester | | | | |
| Alanine | 0.5 | 0.1 | 2.0227 | 0.5042 |
| Glycerine | 0.1 | 0.02 | 0.4031 | 0.0997 |
| Benzalkonium | 0.01 | 0.002 | 0.0409 | 0.0102 |
| Chloride | | | | |
| Ultrapure Water | 75 | 15 | 12.414 | 61.8862 |
| (1st addition) | | | | |
| 1N NaOH | Used to adjust to desired pH | | 0.0653 | 0.0114 |
| 1N HCl | before final water addition | | | |
| Ultrapure Water | qs | qs | 4.9141 | 24.4977 |
| (2nd addition) | | | | |
| Total | 100 | 20 | 20.0594 | |
| Desired pH | Final pH | mOsm/L | | |
| 3.5 | 3.59 | | | |

Formulation # 4

| Lipoic Acid Choline | 1 | 0.2 | 0.2036 | 1.0357 |
|---|---|---|---|---|
| Ester | | | | |
| Alanine | 0.5 | 0.1 | 2.021 | 0.5141 |
| Glycerine | 0.1 | 0.02 | 0.4012 | 0.1013 |
| Benzalkonium | 0.01 | 0.002 | 0.0402 | 0.0102 |
| Chloride | | | | |
| Ultrapure Water | 75 | 15 | 12.3097 | 62.6215 |
| (1st addition) | | | | |
| 1N NaOH | Used to adjust to desired pH | | 0.0289 | 0.0052 |
| 1N HCl | before final water addition | | | |
| Ultrapure Water | qs | qs | 4.6527 | 23.6691 |
| (2nd addition) | | | | |
| Total | 100 | 20 | 19.6573 | |
| Desired pH | Final pH | mOsm/L | | |
| 4 | 4.07 | | | |

| | | | | |
|---|---|---|---|---|
| | | grams | grams | Act. |
| Ingredient | Desired % | needed | used | w/w % |

Formulation # 5

| Lipoic Acid Choline | 1 | 0.2 | 0.2368 | 1.1813 |
|---|---|---|---|---|
| Ester | | | | |
| Alanine | 0.5 | 0.1 | 2.0239 | 0.5048 |
| Glycerine | 0.1 | 0.02 | 0.4016 | 0.0994 |
| Benzalkonium | 0.01 | 0.002 | 0.041 | 0.0102 |
| Chloride | | | | |
| Ultrapure Water | 75 | 15 | 12.3029 | 61.3746 |
| (1st | | | | |
| 1N NaOH | Used to adjust to desired pH | | 0.0129 | 0.0023 |
| 1N HCl | before final water addition | | | |
| Ultrapure Water | qs | qs | 5.0265 | 25.0753 |
| (2nd | | | | |
| Total | 100 | 20 | 20.0456 | |
| Desired pH | Final pH | mOsm/L | | |
| 5 | 5.01 | | | |

Formulation # 6

| Lipoic Acid Choline | 1 | 0.2 | 0.2013 | 1.0040 |
|---|---|---|---|---|
| Ester | | | | |
| Alanine | 0.5 | 0.1 | 2.0216 | 0.5041 |
| Sodium Chloride | 0.25 | 0.05 | 0.5282 | 0.2635 |
| Glycerine | 0.1 | 0.02 | 0.4025 | 0.0996 |
| Benzalkonium | 0.01 | 0.002 | 0.04 | 0.0100 |
| Chloride | | | | |
| Ultrapure Water | 75 | 15 | 11.8086 | 58.8963 |
| (1st addition) | | | | |
| 1N NaOH | Used to adjust to desired pH | | 0.0208 | 0.0036 |
| 1N HCl | before final water addition | | | |
| Ultrapure Water | qs | qs | 5.0268 | 25.0716 |
| (2nd addition) | | | | |
| Total | 100 | 20 | 20.0498 | |
| Desired pH | Final pH | mOsm/L | | |
| 4 | 4.01 | | | |

Formulation # 7

| Lipoic Acid Choline | 1 | 0.2 | 0.2004 | 0.9999 |
|---|---|---|---|---|
| Ester | | | | |
| Alanine | 0.5 | 0.1 | 2.0209 | 0.5042 |
| Sodium Chloride | 0.9 | 0.18 | 1.8409 | 0.9186 |
| Glycerine | 0 | 0 | 0 | 0.0000 |
| Benzalkonium | 0.01 | 0.002 | 0.0402 | 0.0100 |
| Chloride | | | | |
| Ultrapure Water | 75 | 15 | 10.9219 | 54.4961 |
| (1st addition) | | | | |
| 1N NaOH | Used to adjust to desired pH | | 0.009 | 0.0016 |
| 1N HCl | before final water addition | | | |
| Ultrapure Water | qs | qs | 5.0083 | 24.9895 |
| (2nd addition) | | | | |
| Total | 100 | 20 | 20.0416 | |
| Desired pH | Final pH | mOsm/L | | |
| 4 | 4.12 | | | |

Formulation # 8

| Lipoic Acid Choline | 1 | 0.2 | 0.1956 | 0.9784 |
|---|---|---|---|---|
| Ester | | | | |
| Alanine | 0.5 | 0.1 | 2.022 | 0.5057 |
| Sodium Sulfite | 0.05 | 0.01 | 0.2068 | 0.0517 |
| Glycerine | 0.1 | 0.02 | 0.4005 | 0.0994 |
| Benzalkonium | 0.01 | 0.002 | 0.0401 | 0.0100 |
| Chloride | | | | |
| Ultrapure Water | 75 | 15 | 12.16 | 60.8219 |
| (1st addition) | | | | |
| 1N NaOH | Used to adjust to desired pH | | 0.1008 | 0.5042 |
| 1N HCl | before final water addition | | | |
| Ultrapure Water | qs | qs | 4.867 | 24.3438 |
| (2nd addition) | | | | |
| Total | 100 | 20 | 19.9928 | |
| Desired pH | Final pH | mOsm/L | | |
| 4 | 4.03 | | | |

Formulation # 9

| Lipoic Acid Choline | 1 | 0.2 | 0.2205 | 1.0953 |
|---|---|---|---|---|
| Ester | | | | |
| Alanine | 0.5 | 0.1 | 2.027 | 0.5035 |
| Sodium Sulfite | 0.05 | 0.01 | 0.2062 | 0.0512 |
| Glycerine | 0.1 | 0.02 | 0.3996 | 0.0985 |
| Benzalkonium | 0.01 | 0.002 | 0.0392 | 0.0097 |
| Chloride | | | | |
| Ultrapure Water | 75 | 15 | 12.1982 | 60.5944 |
| (1st addition) | | | | |
| 1N NaOH | Used to adjust to desired pH | | 0.0995 | 0.4943 |
| 1N HCl | before final water addition | | | |
| Ultrapure Water | qs | qs | 4.9407 | 24.5429 |
| (2nd addition) | | | | |
| Total | 100 | 20 | 20.1309 | 100.0000 |
| Desired pH | Final pH | mOsm/L | | |
| 4.5 | 4.03 | | | |

Formulation # 10

| Lipoic Acid Choline | 1 | 0.2 | 0.1973 | 0.9873 |
|---|---|---|---|---|
| Ester | | | | |
| Alanine | 0.5 | 0.1 | 2.0249 | 0.5066 |
| Sodium Sulfite | 0.1 | 0.02 | 0.4136 | 0.1035 |
| Glycerine | 0.1 | 0.02 | 0.4013 | 0.0996 |
| Benzalkonium | 0.01 | 0.002 | 0.041 | 0.0102 |
| Chloride | | | | |
| Ultrapure Water | 75 | 15 | 11.9708 | 59.9031 |
| (1st addition) | | | | |
| 1N NaOH | Used to adjust to desired pH | | 0.1921 | 0.9613 |
| 1N HCl | before final water addition | | | |
| Ultrapure Water | qs | qs | 4.7426 | 23.7325 |
| (2nd addition) | | | | |
| Total | 100 | 20 | 19.9836 | |
| Desired pH | Final pH | mOsm/L | | |
| 4.5 | 4.35 | | | |

Formulation # 11

| Lipoic Acid Choline | 1 | 0.2 | 0.2097 | 0.9746 |
|---|---|---|---|---|
| Ester | | | | |
| Alanine | 0.5 | 0.1 | 2.0437 | 0.4749 |
| Sodium Chloride | 0.9 | 0.18 | 1.8022 | 0.8376 |
| Sodium Sulfite | 0.05 | 0.01 | 0.2116 | 0.0492 |
| Glycerine | 0 | 0 | 0 | 0.0000 |
| Benzalkonium | 0.01 | 0.002 | 0.0384 | 0.0089 |
| Chloride | | | | |
| Ultrapure Water | 75 | 15 | 17.0628 | 79.3025 |
| (1st addition) | | | | |
| 1N NaOH | Used to adjust to desired pH | | 0.1477 | 0.0241 |
| 1N HCl | before final water addition | | | |
| Ultrapure Water | qs | qs | | |
| (2nd addition) | | | | |
| Total | 100 | 20 | 21.5161 | |
| Desired pH | Final pH | mOsm/L | | |
| 3.5 | 3.52 | | | |

Formulation # 12

| Lipoic Acid Choline | 1 | 0.2 | 0.1953 | 0.9703 |
|---|---|---|---|---|
| Ester | | | | |
| Alanine | 0.5 | 0.1 | 2.0402 | 0.5068 |
| Sodium Chloride | 0.9 | 0.18 | 1.7998 | 0.8943 |
| Sodium Sulfite | 0.05 | 0.01 | 0.2135 | 0.0531 |
| Glycerine | 0 | 0 | 0 | 0.0000 |
| Benzalkonium | 0.01 | 0.002 | 0.0423 | 0.0105 |
| Chloride | | | | |
| Ultrapure Water | 75 | 15 | 15.6996 | 78.0031 |
| (1st addition) | | | | |
| 1N NaOH | Used to adjust to desired pH | | 0.1362 | 0.0238 |
| 1N HCl | before final water addition | | | |
| Ultrapure Water | qs | qs | | |
| (2nd addition) | | | | |
| Total | 100 | 20 | 20.1269 | |
| Desired pH | Final pH | mOsm/L | | |
| 4 | 4 | | | |

Formulation # 13

| Lipoic Acid Choline | 1 | 0.2 | 0.1997 | 0.9946 |
|---|---|---|---|---|
| Ester | | | | |
| Alanine | 0.5 | 0.1 | 2.0386 | 0.5077 |
| Sodium Chloride | 0.9 | 0.18 | 1.8033 | 0.8982 |
| Sodium Sulfite | 0.05 | 0.01 | 0.2152 | 0.0536 |
| Glycerine | 0 | 0 | 0 | 0.0000 |
| Benzalkonium | 0.01 | 0.002 | 0.0397 | 0.0099 |
| Chloride | | | | |
| Ultrapure Water | 75 | 15 | 15.6858 | 78.1239 |
| (1st addition) | | | | |
| 1N NaOH | Used to adjust to desired pH | | 0.0958 | 0.0168 |
| 1N HCl | before final water addition | | | |
| Ultrapure Water | qs | qs | | |
| (2nd addition) | | | | |
| Total | 100 | 20 | 20.0781 | |
| Desired pH | Final pH | mOsm/L | | |
| 4.5 | 4.48 | | | |

Formulation # 14

| Lipoic Acid Choline | 1 | 0.2 | 0.1976 | 0.9873 |
|---|---|---|---|---|
| Ester | | | | |
| Alanine | 0.5 | 0.1 | 2.0425 | 0.5103 |
| Glycerine | 0.1 | 0.02 | 0.4158 | 0.1031 |
| Benzalkonium | 0 | 0 | 0 | 0.0000 |
| Chloride | | | | |
| Ultrapure Water | 75 | 15 | 17.3172 | 86.5237 |
| (1st addition) | | | | |
| 1N NaOH | Used to adjust to desired pH | | 0.0413 | 0.0073 |
| 1N HCl | before final water addition | | | |
| Ultrapure Water | qs | qs | | |
| (2nd addition) | | | | |
| Total | 100 | 20 | 20.0144 | |
| Desired pH | Final pH | mOsm/L | | |
| 4 | 4.05 | | | |

Formulation # 15

| Lipoic Acid Choline | 1 | 0.2 | 0.215 | 1.0757 |
|---|---|---|---|---|
| Ester | | | | |
| Alanine | 0.5 | 0.1 | 2.0453 | 0.5117 |
| Glycerine | 0.1 | 0.02 | 0.4078 | 0.1012 |
| Benzalkonium | 0 | 0 | 0 | 0.0000 |
| Chloride | | | | |
| Ultrapure Water | 75 | 15 | 17.2993 | 86.5545 |
| (1st addition) | | | | |
| 1N NaOH | Used to adjust to desired pH | | 0.0192 | 0.0034 |
| 1N HCl | before final water addition | | | |
| Ultrapure Water | qs | qs | | |
| (2nd addition) | | | | |
| Total | 100 | 20 | 19.9866 | |
| Desired pH | Final pH | mOsm/L | | |
| 4.5 | 4.52 | | | |

### Example 6:

### Correlation of Ocular Irritation with Percent Micellar LACE Species

FIGS. 12A and 12B generally provide a snapshot of the correlation of irritation to the micellar LACE species over a number of batches compounded.

### Example 7

### Method of Adjustment of Osmolality with Glycerol

The requisite osmolality range for drug-containing formulations and placebo is 280-320 mOsm/kg. Preferably, all LACE formulations need to he within 290-310 mOsm/Kg.
Since LACE has contributions to osmolality, each formulation will have varying concentrations of glycerol to achieve the requisite osmolality.

### I. Summary: Final Adjusted Compositions

TABLE 4
Final Compositions of EV06 Ophthalmic Solutions
with Adjusted Glycerol Concentrations

| | | | Benzalkonium | Actual Total |
|---|---|---|---|---|
| LACE | Glycerol | Alanine | Chloride | Osmolality |
| Conc. | (%) | (%) | (%) | (mOsm/kg) |
| 0% | 2.07% | 0.5% | 0.005% | 295 |
| 1% | 1.56% | 0.5% | 0.005% | 299 |
| 2% | 1.07% | 0.5% | 0.005% | 296 |
| 2.5% | 0.80% | 0.5% | 0.005% | 302 |
| 3.0% | 0.53% | 0.5% | 0.005% | 308 |

### II. Experimental Detail:

A. Glycerol-containing Placebos

A series of placebos was prepared. All placebos, and the LACE-containing solutions that were subsequently prepared, contained the following, with varying amounts of Glycerol:
0.5% (5 mg/g) Alanine
0.005% (0.05 mg/g) Benzalkonium Chloride
Small amounts of 1N Sodium Hydroxide, 1N Hydrochloric Acid, to adjust pH to 4.5
Water for Inhalation (added for final weight)

TABLE 5
Glycerol-containing Placebo (Effect of Glycerol Concentration)

| Glycerol Percent | Osmolality (mOsm/kg) |
|---|---|
| 0.5% | 114 |
| 1.0% | 172 |
| 1.5% | 233 |
| 2.0% | 292 |
| 2.5% | 354 |

B. LACE-Containing Formulations

Based on the standard curve shown in FIG. 12C and the data from fommlations that showed an additional 44-55 mOsm/kg (average of 48 mOsm/kg) for every 1% LACE, a series of solutions was prepared to confirm the actual osmotic contribution of LACE. The target for Total Osmolality was 300 mOsm/kg.

TABLE 6
Glycerol Concentrations for EV06 Compositions

| | Target | | Actual Total |
|---|---|---|---|
| LACE | Osmolality | Glycerol % for | Osmolality |
| Conc. | w/o LACE | target Osm. | (mOsm/kg) |
| 0% | 300 | 2.06% | 295 |
| 1% | 250 | 1.64% | 308 |
| 2% | 203 | 1.25% | 324 |

These data indicate that the effect of LACE on osmolality is somewhat greater than expected, on the order of 57-60 mOsm/kg for every 1%. Accordingly, a full series of solutions was prepared with slightly altered target osmolalities for the solutions without LACE, and therefore different target glycerol contents. All solutions were prepared using the same Alanine/Benzalkonium Chloride, pH 4.5 stock solution used in the placebos, so that the final composition was consistently:
0.5% (5 mg/g) Alanine
0.005% (0.05 mg/g) Benzalkonium Chloride
Small amounts of 1N Sodium Hydroxide, 1N Hydrochloric Acid, to adjust pH to 4.5
Water for Inhalation (added to final weight of 5.0 g per formulation)

TABLE 7
Adjustment of Osmolality of EV06 Compositions

| | | Actual | |
|---|---|---|---|
| | Target | Glycerol % | Actual Total |
| LACE Conc. | Osmolality | used for | Osmolality |
| (Actual %) | w/o LACE | target Osm. | (mOsm/kg) |
| 0% | 300 | 2.06% | 295 |
| 1% (1.01%) | 242 | 1.56% | 299 |
| 2% (2.04%) | 180 | 1.06% | 296 |
| 2.5% (2.51%) | 150 | 0.80% | 302 |
| 3.0% (2.94%) | 120 | 0.56% | 308 |

C. Sterile Preparations

Based on these experimental results, sterile filtered 10.0-g hatches of each formulation were prepared, with the following target compositions, and packaged into sterile eye dropper bottles (2mL per bottle):

TABLE 8
Final Composition Grid of EV06 Compositions

| LACE | | | Benzalkonium |
|---|---|---|---|
| Conc. | Glycerol (%) | Alanine (%) | Chloride (%) |
| 0% | 2.07% | 0.5% | 0.005% |
| 1% | 1.56% | 0.5% | 0.005% |
| 2% | 1.07% | 0.5% | 0.005% |
| 2.5% | 0.80% | 0.5% | 0.005% |
| 3.0% | 0.53% | 0.5% | 0.005% |

### Example 8

### Method of Preparation of LACE Chloride Pharmaceutical Compositions

A method of preparing LACE pharmaceutical composition is as follows:
At room temperature, Water for Injection (WFI) at 80% of batch weight is added to glass compounding vessel. The water is purged with nitrogen to achieve: S10 ppm oxygen.
Stepwise, alanine, glycerin, and BAK, are added, and mixed until dissolved.

The pH is adjusted to 4.4-4.6 with HCl or NaOH.
LACE is ground in a mortar and pestle under nitrogen to de-chimp and slowly added while mixing.
Deoxygenated Water for Injection is added to achieve final batch target weight.
Batch is mixed for a total of 8 hours to ensure complete dispersion and dissolution.
The pH may be adjusted to 4.4-4.6 with NaOH or HCl if needed.
Osmolality may adjusted to 290-310 with glycerol if needed.

After 8 hours of mixing, EV06 bulk drug product solution is aseptically filtered through a capsule SHC 0.5/0.2 μm sterilizing filter into a holding bag.
The bulk product solution in the holding bag is kept at 5° C. by refrigeration or ice bath.
Filter bubble point test is performed to ensure the integrity of the filter.

Sterile filtered bulk solution is aseptically transferred to the Class 100 room and filled into pre-sterilized bottles.
Sterile tips and caps are applied to the bottles under nitrogen overlay.
Sealed bottles are transferred to trays, which are bagged with a nitrogen purge and immediately transferred to 5° C. storage.

### Example 9

### Stability Studies of LACE Chloride Formulations

Early formulation prototypes contained sodium edetate and 0.01% benzalkonium chloride. Stability studies with and without these excipients demonstrated that sodium edetate did not stabilize LACE. Presence of excess benzalkonium chloride slightly destabilized the drug. Thus, the final formulation contains no sodium edetate and 0.005% benzalkonium chloride. Through microbiological testing, 0.004% benzalkonium chloride in the current formulation composition was shown to be effective as a preservative in the drug product.

in an effort to stabilize the drug formulation further, systematic stability studies (5° C., 25° C. and 40° C.) on mid-scale R&D batches were undertaken with bottled EV06 Ophthalmic Solution in the presence and absence of oxygen scavenging packets contained in zip-lock, vapor impermeable foil pouches. Bottles of product stored at 5° C. in the presence of an oxygen scavenging packet sealed in re-sealable foil pouches demonstrated stability at 12 months.

Additional precautions were implemented throughout the development process to stabilize the final formulation from degradation due to exposure to environmental oxygen and non-refrigerated conditions. Handling of the drug substance under nitrogen (exclusion of oxygen and minimization of moisture) and compounding under a nitrogen blanket were implemented to minimize exposure to oxygen. After compounding, the product is filled into a vapor impermeable holding bag and stored under refrigerated conditions until bottling ensues. The holding bag containing the bulk solution is kept cold during filling. A nitrogen blanket is placed over the drug solution in each bottle, to minimize oxygen exposure.

TABLE 9
Supporting Stability- Batch ECV-12 JUN. 2014-120-04
Stability Table for EV06 Ophthalmic Solution, 3.0%
Container Polyethylene dropper bottle, 6 cc
Secondary Container: Foil Pouch
Closure: Dropper Tip and Cap
Oxygen Adsorbent: Oxygen Adsorbent Packet present

| | | | | | |
|---|---|---|---|---|---|
| | | Acceptance | | | |
| Test | Method | Criteria | T = 0 | 2 Weeks | 1 Month |

5^{°} C.

| Appearance | ATM-1095 | Clear, pale yellow | Conforms | | Conforms | | Conforms | |
|---|---|---|---|---|---|---|---|---|
| | | to yellow solution | | | | | | |
| | | essentially free of | | | | | | |
| | | foreign or | | | | | | |
| | | particulate matter | | | | | | |
| Assay, | ATM-1405 | 90.0-110.0% of | 101.1% | | 107.1% | | 106.7% | |
| LACE | | Label Claim | | | | | | |
| Related | ATM-1405 | Individual | RRT 0.57: 0.05% | | RRT 0.58: 0.17% | | RRT 0.59: 0.21% | |
| Compounds | | Report 2: 0.05% | RRT 0.59: 0.21% | | RRT 0.63: 0.09% | | RRT 0.64: 0.09% | |
| | | (% area) | RRT 0.64: 0.13% | | RRT 0.66: 0.17% | | RRT 0.67: 0.15% | |
| | | Total Report | RRT 0.83: 0.06% | | RRT 0.83: 0.06% | | RRT 0.83: 0.06% | |
| | | | RRT 1.23: 0.14% | | RRT 1.23: 0.16% | | RRT 1.18: 0.05% | |
| | | | Total: 0.8% | | LA: 0.06% | | RRT 1.20: 0.10% | |
| | | | | | Total: 0.7% | | LA: 0.10% | |
| | | | | | | | Total: 0.8% | |
| Assay, | ATM-1406 | Report | 0.0447 | mg/mL | 0.0443 | mg/mL | 0.0446 | mg/mL |
| preservative | | | | | | | | |
| pH | USP <791> | Report | 4.6 | | 4.5 | | 4.5 | |
| Osmolality | USP <785> | 250-350 mOsm/kg | 262 | mOsm/kg | 263 | mOsm/kg | 263 | mOsm/kg |

25° C. ± 5°

| Appearance | ATM-1095 | Clear, pale yellow | | Conforms | | Conforms | | Conforms | |
|---|---|---|---|---|---|---|---|---|---|
| | | to yellow solution | | | | | | | |
| | | essentially free of | | | | | | | |
| | | foreign or | | | | | | | |
| | | particulate matter | | | | | | | |
| Assay, | ATM-1405 | 90.0-110.0% of | | 101.1% | | 107.3% | | 107.0% | |
| LACE | | Label Claim | | | | | | | |
| Related | ATM-1405 | Individual | | RRT 0.57: 0.05% | | RRT 0.56: 0.05% | | RRT 0.59: 0.27% | |
| Compounds | | Report 2: 0.05% | | RRT 0.59: 0.21% | | RRT 0.58: 0.22% | | RRT 0.64: 0.12% | |
| | | (% area) | | RRT 0.64: 0.13% | | RRT 0.63: 0.12% | | RRT 0.67: 0.18% | |
| | | Total Report | | RRT 0.67: 0.21% | | RRT 0.66: 0.21% | | RRT 0.33: 0.07% | |
| | | | | RRT 0.83: 0.06% | | RRT 0.83: 0.07% | | RRT 1.20: 0.16% | |
| | | | | RRT 1.23: 0.14% | | RRT 1.23: 0.09% | | RRT 1.85: 0.09% | |
| | | | | Total: 0.8% | | RRT 1.25: 0.07% | | LA: 0.65% | |
| | | | | | | LA: 0.35% | | Total: 1.5% | |
| | | | | | | Total: 1.2% | | | |
| Assay, | ATM-1406 | Report | | 0.0447 | mg/mL | 0.0445 | mg/mL | 0.0447 | mg/mL |
| preservative | | | | | | | | | |
| pH | USP <791> | Report | | 4.6 | | 4.4 | | 4.4 | |
| Osmolality | USP <785> | 250-350 mOsm/kg | | 262 | mOsm/kg | 264 | mOsm/kg | 264 | mOsm/kg |
| | Test | 2 Month | | 3 Month | | 6 Month | | 12 Month | |
| | 5^{°} C. | | | | | | | | |
| | Appearance | Conforms | | Conforms | | Conforms | | Conforms | |
| | Assay, | 98.9% | | 98.1% | | 95.0% | | 94.7% | |
| | LACE | | | | | | | | |
| | Related | RRT 0.61: 0.10% | | RRT 0.65: 0.08% | | RRT 0.61: 0.34% | | RRT 0.59: 0.07% | |
| | Compounds | RRT 0.66: 0.07% | | RRT 0.67: 0.15% | | RRT 0.67: 0.21% | | RRT 0.63: 0.12% | |
| | | RRT 0.86: 0.07% | | RRT 0.71: 0.12% | | RRT 0.69: 0.23% | | RRT 0.68: 0.09% | |
| | | RRT 1.21: 0.08% | | RRT 0.74: 0.16% | | RRT 0.84: 0.06% | | RRT 0.72: 0.12% | |
| | | RRT 1.27: 0.09% | | RRT 0.83: 0.05% | | RRT 1.14: 0.08% | | RET 0.89: 0.05% | |
| | | LA: 0.17% | | RRT 1.09: 0.07% | | RRT 1.20: 0.10% | | RRT 1.38: 0.06% | |
| | | Total: 0.6% | | RRT 1.15: 0.09% | | LA: 0.32% | | RRT 1.42: 0.09% | |
| | | | | LA: 0.22% | | Total: 1.3% | | LA: 0.38% | |
| | | | | Total: 0.9% | | | | Total: 1.0% | |
| | Assay, | 0.0447 | mg/mL | 0.0440 | mg/mL | 0.0441 | mg/mL | 0.0504 | mg/mL |
| | preservative | | | | | | | | |
| | pH | 4.5 | | 4.5 | | 4.4 | | 4.3 | |
| | Osmolality | 262 | mOsm/kg | 262 | mOsm/kg | 261 | mOsm/kg | 255 | mOsm/kg |

| 25° C. ± 5° | | | | | | |
|---|---|---|---|---|---|---|
| Appearance | Conforms | | Conforms | | Conforms | |
| Assay, | 97.2% | | 98.9% | | 87.1% | |
| LACE | | | | | | |
| Related | RRT 0.59: 0.18% | | RRT 0.66: 0.28% | | RRT 0.61: 0.58% | |
| Compounds | RRT 0.66: 0.09% | | RRT 0.72: 0.15% | | RRT 0.69: 0.30% | |
| | RRT 0.86: 0.07% | | RRT 0.74: 0.12% | | RRT 0.84: 0.06% | |
| | RRT 1.21: 0.08% | | RRT 0.83: 0.06% | | RRT 1.14: 0.08% | |
| | RRT 1.27: 0.19% | | RRT 1.09: 0.07% | | RRT 1.20: 0.29% | |
| | LA: 0.94% | | RRT 1.15: 0.20% | | RRT 1.26: 0.51% | |
| | Total: 1.6% | | RRT 1.21: 0.27% | | RRT 1.34: 0 18% | |
| | | | RRT 1.27: 0.09% | | LA: 1.18% | |
| | | | LA: 1.05% | | Total: 3.2% | |
| | | | Total: 2.4% | | | |
| Assay, | 0.0438 | mg/mL | 0.0435 | mg/mL | 0.0462 | mg/mL |
| preservative | | | | | | |
| pH | 4.3 | | 4.2 | | 4.1 | |
| Osmolality | 263 | mOsm/kg | 261 | mOsm/kg | 259 | mOsm/kg |

### Related Compounds: LA=<-R-Lipoic Acid (USP Standard)

### Example 10

### Formulation Studies to Disrupt Micellization of LACE Iodide

### Summary of Experiments:

In experiments where Sodium Chloride was either added to an existing LACE-Iodide formulation, or a solution containing Sodium Chloride was used to dissolve the LACE-Iodide API, the “associative species” peak was not significantly decreased.

In experiments where a co-solvent such as Ethanol or Propylene Glycol was used to suspend the API prior to addition of an aqueous vehicle, there was a very significant reduction in the percentage of the associative species. Addition of an organic solvent to an existing formulation also decreased the associative species peak, to a lesser extent.

These results point to formulation strategies that can interfere with the hydrophobic interaction between LACE molecules as a means of controlling the associative species.

### Background

The “associative species” that we have observed by RP-HPLC, which represents a large percentage of the API in the various formulated hatches prepared using the LACE-Iodide, has been hypothesized to he a micellar aggregate. This is based in part on the surfactant-like structure of the LACE molecule, and the ability to dissipate this species by dilution or additional stirring in the case of the LACE-Chloride.

In the literature, Sodium Chloride is a known micelle disruptor. Therefore a series of experiments was undertaken to test whether this “associative species” could he dissipated by addition of sodium chloride, or other ingredients that would be expected to disrupt the associative species by other mechanisms, such as hydrophobic interactions.

### Results

As a first test of this hypothesis, an existing formulation (batch FK-LACE-02-15) known to exhibit a large “associative species” peak was mixed with solutions containing various levels of Sodium Chloride (NaCl). The final diluted LACE concentration was targeted to the level appropriate for RP-HPLC analysis (12.8 mg/mL of LACE-Iodide).

Table 10 shows the key results of this set of experiments, which did not demonstrate any significant change in ⁻the level of associative species over time, even at levels of salt (NaCl) far above what would be acceptable in the eye (due to very high osmolality).

Diluting the formulation with Acetonitrile to the same final LACE-Iodide concentration, resulting in —33% Acetonitrile overall, led to a modest decrease in the level of associative species, from a range of 36-40% down to 26% in 4 hours.

TABLE 10
Addition of Salt or Organic Solvent to Batch FK-LACE-02-15

| Condition (ingredient listed | % Associative Species at: | | |
|---|---|---|---|
| with final concentration) | T = 0 | 4 hou s | 24 hours |
| Formulation as is | 39.0% | | |
| (dil. to 12.8 mg/g) | | | |
| Form. + 0.9% NaCl | 37.1% | 38.3 | 37.2% |
| Form. + 1.8% NaCl | 35.9% | (not meas.) | 36.4% |
| Form. + 3.6% NaCl | 36.2% | 36.0 | (not meas.) |
| Formulation + | 33.4% | 25.8 | 23.7% |
| 33% Acetonitrile | | | |

In the next set of experiments, the LACE-Iodide API was dissolved in various ways to determine whether these conditions could prevent the initial formation of the associative species, and therefore eliminate the seed that allowed further growth of this species over time. The conditions tested were:

Dissolution in pH 4.5 buffer (0.5% Alanine, 0.005% BAK) containing 1.8% NaCl.

Dissolution in Ethanol—API did not dissolve in neat Ethanol, forming a suspension. About 22% by volume of the aqueous pH 4.5 buffer was added, leading to nearly complete dissolution of the API, with some heating at 37° C.

Dissolution/suspension in Propylene Glycol, followed by dissolution in the aqueous pH 4.5 buffer. Propylene Glycol was added first, and represented 10% by weight of the final solution.

Dissolution in pH 4.5 buffer containing 0.6% NaCl and 1.5% Propylene Glycol (PG). This was intended to test whether disruption of charge-charge interactions (by NaCl) and hydrophobic interactions (by PG) would have a synergistic effect, using concentrations of each that would he reasonable in terms of osmolality,

As shown in Table 10, the Ethanol and Propylene Glycol experiments were successful in eliminating or significantly reducing the associative species present at T=0, relative to the other dissolution experiments. Note that the solution was added to the API powder, rather than the formulation practice of adding API to solution, which may explain why T=0 was high in some of these cases, but not on the day the formulated hatches were prepared.

TABLE 10
Experiments with Direct Dissolution of
LACE-Iodide API (Lot# 011510)

| Dissolution Method | % Associative Species at T0 |
|---|---|
| Buffer with 1.8% NaCl | 34.7% |
| Ethanol (78% final), | 0.0% |
| then Buffer (22%) | |
| Propylene Glycol (10%), | 11.7% |
| then Buffer (90%) | |
| Buffer with 0.6% NaCl, 1.5% PG | 40.9% |

### Example 11

### Formulation Studies with Cyclodextrins to Disrupt Micellization of LACE Iodide Hypothesis

Associative species can be mitigated by inclusion of excipients that interfere with hydrophobic interactions between LACE molecules.

Formulations containing Polypropylene Glycol, Dexolve-7 (Sulfobutylether-beta-cyclodextrin), or Hydroxypropyl-beta-cyclodextrin were prepared and analyzed for associative species and related substances.

TABLE 11
Formulation Studies to Prevent Miceliization of LACE Iodide in Solution

| Batch ID, | | | Osmolality | % Associative | | % Related | % Oxo-LACE |
|---|---|---|---|---|---|---|---|
| Composition | Batch Size | | (mOsm/kg) | Species at 5C | | Substances | Species |
| FK-LACE-02-09 | 160 | g | 278 | 32.9% | (4 weeks) | 1.33% | 0.83% |
| Control batch | | | | | | | |
| Same as LACE-Cl | | | | | | | |
| formulation | | | | | | | |
| FK-LACE-02-28 | 3 | g | 281 | 0.0% | (24 h) | TBD | Not visible by |
| 5.76% Dexolve | | | | | | | RP-HPLC |
| FK-LACE-02-29 | 3 | g | 299 | 0.0% | (72 h) | 6.69% | 4.66% |
| 5% PPG | | | | | | | |
| FK-LACE-02-32 | 25 | g | 279 | 4.5% | (24 h) | 0.94% | 0.21% |
| Same as Control | | | | 3.6% | (96 h) | | |
| FK-LACE-02-33 | 25 | g | 273 | 13.2% | (24 h) | 5.67% | 5.15% |
| 5% PPG | | | | 14.2% | (96 h) | | |
| FK-LACE-02-36 | 25 | g | 281 | 0.0% | (72 h) | 0.98% | 0.09% |
| 5.76% Dexolve | | | | | | | |
| FK-LACE-02-37 | 25 | g | 151* | 0.0% | (72 h) | 0.81% | 0.11% |
| 5.0% HPCD | | | | | | | |

% oxo-LACE species are shown at T = 0.

### Example 12

### Enhanced Stabilily in HP-B-CD/Lace-Iodide Formulations

These experiments demonstrate enhancement of stability achieved by HP-B-CD/Lace-Iodide formulation compared to non-HP-B-CD/Lace-Iodide formulation.

Experiment #1

Formulations were prepared that comprised 3% LACE-Iodide either with (16.1% HPBCD) or without Hydroxypropyl-B-cyclodextrin (HP-B-CD) at a 10-g scale. Both formulations contained 0.5% Alanine, pH 4.5, 50 ppm Benzalkonium Chloride, and Glycerol for osmolality adjustment and all solutions were at pH 4.2-4.5. In the formulation that contained HP-B-CD, the cyclodextrin was present in a 1.5:1 molar ratio, relative to the LACE concentration. The formulations were filtered through a 0.2-μm PVDF membrane, and 5 mL of each formulation was filled into a 10-mL LDPE eye dropper bottle, and then blanketed with nitrogen before the dropper tip was inserted and the bottle capped. The eye-dropper bottle was not barrier pouched at the time of filling.

The eve dropper bottles with the two formulations were stored at 25° C. in a temperature-controlled incubator, and 0.5 mL (about 10 drops) sampled at each time point for analysis of related substances (by HPLC). The nitrogen blanket was not replenished, so some air got into the bottle with each sampling. This experiment was an early investigation of stability at room temperature (25±0.1° C.) with no protection from oxygen with continued sampling.

FIG. 18 shows a time course of the increase of the oxidized species of LACE over 20 days at 25C with repeated sampling (square: LACE-I, 3% formulation, 16.1% HP-B-CD; diamond: LACE-I, 3% formulation, no HP-B-CD). The sampling time-points were T-0, I day, 2 days, 8 days, 12 days and 17 days).

These data (FIGS. 18 and 19) demonstrated that the cyclodextrin protected LACE from oxidation both initially, resulting in lower amounts of oxidized API during preparation, as well as under an accelerated stress condition with increasing amounts of oxygen present. The formulation with HP-B-CD remained within the specification of ≤2.0% total impurities through 17 days (not including Lipoic Acid) under these conditions. At the end of 20 days, lipoic acid concentration was —0.20%.

### Example. 13

### Comparative Stability between LACE-Chloride Clinical Formulation and LACE-Iodide HP-B-CD

For the stability studies on both the clinical LACE-Chloride and the prototype LACE-Iodide formulation with a 1:1 molar ratio of HP-B-CD to LACE, the formulations were filtered, filled into LDPE eye dropper bottles, blanketed with nitrogen, and then placed inside barrier foil pouches with an oxygen scavenger. It is likely that some oxygen was still present in the pouch to start. After the first time point following T=0, however, the rise in oxidized LACE species stops, even at elevated temperatures, likely due to depletion of the remaining oxygen (FIG. 20). The rate of increase of oxidized species for LACE-Chloride was slightly higher at 25C than at 5C, though not significantly.

The prototype LACE-Iodide formulation containing HP-B-CD shows lower levels of oxidized LACE to start (˜0.11% for LACE-Iodide, as opposed to 0.3% for LACE-Chloride), despite being prepared without any nitrogen blanket during dissolution of the API. For the clinical LACE-Chloride formulation, the solution was deoxygenated and a nitrogen blanket was maintained during dissolution.

In addition, after being blanketed with nitrogen and placed inside the pouch, the prototype LACE-Iodide formulation displayed a much smaller rise in the total Oxidized LACE percentage before leveling off. The extent of the initial rise was dependent on temperature for both formulations. This allowed for estimation of the activation energy for each formulation by ,Arrhenius modeling. For the prototype LACE-Iodide formulation with HP-B-CD, the activation energy was more than tripled relative to the original LACE-C1 formulation (FIG. 21), further indicating that HP-S--CD stabilizes LACE against oxidation.

| Formulation | Activation Energy |
|---|---|
| Clinical LACE-Cl | 9.9 kJ/mol |
| Prototype LACE-I w/HPbCD | 33.2 kJ/mol |

Activation energies for the hydrolysis mechanism of LACE degradation, which results in growth of Lipoic Acid, were also calculated from the stability data (FIG. 22). In contrast to the oxidation mechanism, the activation energies for hydrolysis for both the LACE-C1 formulation and the LACE-I fommlation were similar (65.6 kJ/mol and 69.4 kJ/mol, respectively) (FIG. 21), indicating that the cyclodextrin has no significant impact on hydrolysis.

### Example 14

### Corneal Permeability Studies of LACE-Chloride and LACE-Iodide

A critical question was whether the drug formulated with hydroxypmpyl beta cyclodextrin (HP-B-CD) permeated corneal tissue adequately and was accessible to corneal esterases to release the active drug, lipoic acid. As mentioned earlier, Lipoic Acid is the active drug for this indication: Presbyopia.

The experiments below tested: (a) the permeability of lipoic acid choline ester (LACE) through bovine calf cornea via LACE-Iodide formulations containing hydroxypropyl-B-cyclodextrin (HP-B-CD) at different concentrations, and (b) comparative permeability of LACE-Chloride versus LACE-Iodide. The experiments were performed using a Franz Cell Diffusion apparatus shown in FIG. 23.

LACE is delivered from these formulations as one of two salts: LACE-chloride and LACE-iodide. LACE is the pro-drug, traveling through the corneal barrier before being hydrolyzed into lipoic acid, the active drug, through the action of ocular esterases and through passive hydrolysis of the drug compound at physiological conditions. Therefore, both LACE and lipoic acid concentrations were assayed at each time point to evaluate permeability.

### Compositions for Corneal Permeability, by Study #

| | Study 1 | | Study 2 | | Study 3 | | Study 4 | |
|---|---|---|---|---|---|---|---|---|
| | AC- | ECV- | AC- | AC- | AC- | ECV- | AC- | FK- |
| | LACE- | 23 Apr. 15- | LACE- | LACE- | LACE- | 23 Apr. 15- | LACE- | LACE- |
| (% w/w) | 03-33 | 112-08 | 03-36 | 03-39 | 03-39 | 112-08 | 03-33 | 02-32 |
| LACE-I | 1.92 | 1.5% | 3.0 | 4.5 | 4.5 | 1.5% | 1.92 | 1.92 |
| | | LACE-Cl | | | | LACE-Cl | | |
| HP-JJ-CD | 6.88 | — | 10.75 | 16.12 | 16.12 | — | 6.88 | — |
| Alanine | 0.5 | 0.5 | 0.5 | 0.5 | 0.5 | 0.5 | 0.5 | 0.5 |
| BAK | 0.005 | 0.005 | 0.005 | 0.005 | 0.005 | 0.005 | 0.005 | 0.005 |
| Glycerol | 1.1 | 1.37 | 0.6 | 0.0 | 0.0 | 1.37 | 1.1 | 1.38 |
| WFI | 89.6 | 96.6 | 85.15 | 78.88 | 78.88 | 96.6 | 89.6 | 96.2 |

### Procedure:

a. 6 bovine eyeballs are dissected in a sterile laminar flow hood.
b. The corneas are extracted from the eyeball, briefly rinsed in sterile double-distilled water, and submerged in 3 mL of glutathione buffer (0.1% glutathione, oxidized, 6 mM sodium phosphate, pH 7, sterile-filtered) in a sterile culture dish.
c. The corneas are kept at 5° C. and used within 24 hours of excision.
d. Six 5 mL Franz vertical diffusion cells are cleaned with distilled water and isopropanol and air dried in a laminar flow cabinet prior to set up.
e. A small stir bar is placed within the receptor fluid chamber. The bottle of receptor fluid (5 mM phosphate-buffered saline with 0.1% Tween 20, pH 7.4, sterile-filtered) is tared on an analytical balance, and 4.5 mL of it is added to each Franz cell. The exact weight of the starting receptor fluid is recorded.
f. The cornea is gently rinsed of glutathione buffer with receptor fluid, and is placed on the donor pedestal. The donor chamber is placed on top of the cornea, and the entire assembly is fastened to the pedestal with a metal clip. At this point, 0.5 mL of additional receptor fluid is added via the sampling arm, until the fluid level reaches the point marked on the arm with a black line. The weight of this addition is also recorded.
g. The Franz diffusion apparatus is connected to a heater unit, and the temperature is raised to 37° C. When that temperature is reached, the formulation (“the donor solution”) is added to the donor chamber.
h. 0.2 mL of donor solution is added. Both the donor chamber and sampling arm are covered by paraffin when not in use to prevent evaporation.
i. Sampling is done via Drummond pipet, and only through the sampling arm. 200-300 μL of receptor fluid is sampled from each cell at each time point.
j. The sample is added to an amber glass HPLC vial with 0.3 mL glass insert, and is weighed. The volume taken from the sampling arm is replaced with fresh receptor fluid.
k. When sampling, the fluid level was never allowed to fill below the start of the sampling arm, such that air bubbles were introduced to the receptor chamber. If the fluid had evaporated significantly between two time points, a pre-sampling replacement was added and recorded, and sampling proceeded as normal.
l. The samples were stored at 5° C., until HPLC analysis of assay.
m. Corneas were extracted with bead mill homogenization.

Study 1: The purpose of this study was to compare the permeability of AC-LACE-03-33, containing 1.92% LACE-Iodide, with ECV-23 Apr. 15-112-08, Demo #6 (Frontage, 1.5% LACE-Chloride), in order to evaluate the effects of HP-B-CD on the passage of LACE through the cornea. Given the difference in molecular weight between LACE-I and LACE-Cl, these were equivalent concentrations of LACE. Thus, a 1.5% LACE-Chloride was equivalent to a 1.92% LACE-Iodide formulation. No esterase inhibitor was used in the experiment.

The results from Study 1 (FIGS. 24 and 25) demonstrated that the majority of the drug product that permeated was lipoic acid, which had been hydrolyzed from LACE during passage through the cornea, or in the receptor solution prior to time point collection. The permeated species was almost entirely lipoic acid for the LACE-Iodide formulation, with somewhat more intact LACE permeated with the LACE-Chloride formulation. This is somewhat expected due to the larger ionic size and molecular weight of the LACE-Iodide molecule, compared to the LACE-Chloride molecule, possibly resulting in a longer residence time in the cornea and a higher degree of hydrolysis to lipoic acid. Permeates were analyzed immediately after collection after each sampling point. The overall percent of drug permeated was similar between LACE-I and LACE-Cl-containing formulations, at 3-7° (not including one high-permeation outlier for the LACE-D.

Study 2: The purpose of this study was to evaluate the permeability of two LACE-I formulations, with different concentrations of LACE-I: AC-LACE-03-36 (3% LACE-Iodide/10.7% HP-B-CD) and AC-LACE-03-39 (4.5% Lace-Iodide/16.1% HP-B-CD) (FIGS. 26 and 27).

The results from Study 2 showed that most of the permeated drug existed in the receptor fluid in its lipoic acid form, but in lower concentrations compared to the previous study, despite there being higher drug concentrations. A significant portion of the drug was contained within the corneal tissue due to the crop of thicker calf corneas (˜1.5-1.8 mm in Study 2, ˜0.6-0.8 mm in Study 1) available for this study. A range of 1-5% of the total amount of lipoic acid was extracted from the corneal tissue, with an average of 3.4% extracted from the corneas exposed to AC-LACE-03-36 (3.0% LACE-I/10.7% HP-B-CD) and 2.5% extracted from the corneas for AC-LACE-03-39 (4.5% LACE-I/16.1% HP-B-CD).

Study 3: This study investigated permeability between a LACE-Iodide formulation that contained HP-B-CD and a LACE-Chloride formulation that contained no HP-B-CD. The purpose of this study was to build on previous data obtained in Study 2, by examining the difference in LACE conical permeability between AC-LACE-03-39 (4.5% LACE-I/16.1% HP-B-CD) and ECV-23 Apr. 15-112-08 (1.5% LACE-Cl, no HP-B-CD) to further determine whether the concentration of LACE was an impediment to its permeation across the corneal layer.

Extraction of LACE/LA from the conical section of contact was done by bead mill homogenization, and revealed that a higher mass of Lipoic Acid was found in the corneal tissue exposed to AC-LACE-03-39 (4.5% LACE-I/16.1% HP-B-CD) upon conclusion of the study, although the increase in concentration within the corneas was significantly smaller than the increase in delivered API concentration (FIGS. 28 and 29). Therefore the highest dose of 4.5% LACE-I may not provide a significant advantage in terms of permeated drug.

Study 4: This study compared the effect of hydroxypropyl beta cyclodextrin on permeability, while keeping the LACE salt form constant. In this study, both cohorts were LACE-Iodide.

The formulations were FK-LACE-02-32 (1.92% LACE-I, no HP-B-CD) and AC-LACE-05-21B (1.92% LACE-I, 1 molar equivalent HP-B-CD (7.4%)). The purpose of this study was two-fold. The first objective was to directly compare two LACE-I solutions, of equal concentrations, such that HP-B-CD's impact on permeation would be directly examined. The second objective was to examine HP-B-CD's impact on retention of the drug product within the corneal tissue.

The data of this study indicates that HP-B-CD has no impact on conical retention of the drug—for both formulations, 7% of the total LA (lipoic acid) on average was extracted from the corneal sections (FIGS. 30 and 31).

In terms of permeation across the corneal layer, all 3 corneas for FK-LACE-02-32 showed permeation from 4-6 hours onward, while only 1 cornea for AC-LACE-05-2.1B showed permeation starting at the 4-hour time point. However, the average permeated drug product at 28 hours was similar, with 12.67±5.62% of total LA for FK-LACE-02-32 and 11.27±9.78% of total LA for AC-LACE-5-21B. The similarity in extracted corneal concentrations, as well as the similar average permeation at 28 hours shows that HP-B-CD is not an impediment toward LACE-I entering the corneal tissue.

All data assessed together, demonstrates that LACE-Iodide can be administered to the ocular surface with no impediment of transport due to its larger molecular size and the delivery system (HP-B-CD). Additionally, study results demonstrated efficient transport of LACE through the cornea at all concentrations investigated. Furthermore, high lipoic acid concentrations produced in the receptor fluid for LACE-Iodide/HP-B-CD concentrations demonstrated conversion of LACE to lipoic acid by corneal esterases. LACE-Chloride in contrast, showed more of a mixture of lipoic acid and LACE, possibly due to its lower molecular weight.

### Example 15

### Associative Species as a Function of the Molar Ratio of LACE-I: HP-B-CD

Previous experiments demonstrated that Hydroxypropyl Beta Cyclodextrin (HP-B-CD) could disrupt micellization of LACE-I in aqueous solution. These experiments determine the molar ratio of LACE-Iodide to Hydroxypropyl Beta Cyclodextrin (HP-B-CD required to generate thermodynamically stable inclusion complexes.

| | |
|---|---|
| HP-β-CD + Lace- | [symbol] (HP-β-CD − Lace-Iodide] (Complete Inclusion |
| HP-β-CD + Lace- | [symbol] (HP-β-CD − Lace-Iodide] + |
| | HP-β-CD (Complete Inclusion |
| HP-β-CD + Lace- | [symbol] (HP-β-CD − Lace-Iodide] + |
| | Lace-Iodide (Partial Inclusion |

The approach was to generate complete inclusion complexes of LACE-Iodide in HP-B-CD, thus preventing any opportunity of aggregation of LACE molecules. Several batches of formulation were prepared using varying molar ratios of LACE-Iodide to HP-B-CD and the growth of aggregative species assessed over time. The formulations were stored at 5° C. The formation of associative species as measured by reverse phase HPLC was then reported as the area percent relative to the main LACE peak area.

The results established that the formation of associative species could be prevented when there was at least a one to one molar equivalence between the concentration of LACE-I and HP-B-CD (as shown in FIG. 32).

### Example 16

### Correlation between Aggregative Species and In-Vivo Ocular Irritation

Example 16 established the correlation between concentration of associative species and ocular irritation in an in-vivo model (rabbit Draize model). The data showed that average irritation scores of 0-0.5 could be obtained when the molar equivalent ratio of LACE-Iodide:HP-B-CD was 1:1 or 1:1.5.

TABLE 12
Correlation between Associative Species and Ocular Irritation

| | | | | | Associative | Ave. |
|---|---|---|---|---|---|---|
| | % LACE-I | | Molar Ratio | Associative Species | Species | Irritation |
| Batch | (w/w) | Cyclodextrin | CD to API | (Area % vs. LACE) | (% in. solution.)* | Scores |
| FK-LACE-02-09 | 1.92 | None | N/A | 39% | 0.75% | 4.25 |
| AC-LACE-03-18 | 1.92 | HP-y-CD | 1.0 | 6.4% | 0.12% | 0 |
| | | (6.0%) | | | | |
| AC-LACE-03-10C | 4 | HP-B-CD | 0.54 | 8.4% | 0.34% | 4 |
| | | (7.8%) | | | | |
| AC-LACE-03-10D | 4 | HP-B-CD | 0.35 | 24.9% | 1.0% | 6 |
| | | (5.0%) | | | | |
| AC-LACE-03-33 | 1.92 | HP-B-CD | 1.0 | 0.5% | 0.01% | 0 |
| | | (7.4%) | | | | |
| AC-LACE-03-36 | 3 | HP-B-CD | 1.0 | 1.3% | 0.04% | 0.5 |
| | | (10.7%) | | | | |
| AC-LACE-03-39 | 4.5 | HP-B-CD | 1.0 | 0.3% | 0.014% | 0 |
| | | (16.1%) | | | | |
| AC-LACE-03-54 | 3 | HP-B-CD | 1.5 | 0.6% | 0.02% | 0 |
| | | (16.1%) | | | | |
| FK-LACE-02-36 | 1.92 | Dexolve | ~0.8 | BLOQ | 0.0% | 0 |
| | | (5.8%) | | | | |

*Calculated by multiplying LACE-I concentration by Area % of Associative Species

### Example 18

### Summary of Stability Studies Of LACE-Iodide/HPBCD

| Study # | Purpose/Design | Lot# | Start Date |
|---|---|---|---|
| N/A | Effect of cyclodextrins vs. none; | FK-LACE-02-32 | 13 Apr. 2016 |
| | Conditions: 5 C., 25 C. | FK-LACE-02-36 | 13 Apr. 2016 |
| | Conditions: Glass vials, no pouch, | FK-LACE-02-37 | 13 Apr. 2016 |
| | nitrogen overlaid | | |
| 052516 | Effect of cyclodextrins vs. none; | AC-LACE-03-54 | 25 May 2016 |
| | Conditions: 25 C., simulated end-use | | |
| | LDPE, pouched, nitrogen overlaid, | AC-LACE-03-56 | 25 May 2016 |
| | oxygen scavengers | | |
| 0205 | Effect of LACE-1 concentration and | AC-LACE-05-21 | 1 Jul. 2016 |
| | different HPbCD/LACE-I ratios; | AC-LACE-05-21B | 1 Jul. 2016 |
| | Conditions: 5 C., 25 C., 40 C. | AC-LACE-05-23 | 1 Jul. 2016 |
| | LDPE, pouched, nitrogen overlaid, | AC-LACE-05-23B | 1 Jul. 2016 |
| | oxygen scavengers | | |
| 0214 | Stability on batch prepared without | AC-LACE-05-39 | 12 Jul. 2016 |
| | N2 purge during preparation; | | |
| | Conditions: 5 C., 25 C., 40 C. | | |
| | LDPE, pouched, nitrogen overlaid, | | |
| | oxygen scavengers | | |
| 0225 | Stability on batch prepared with 0.23% | AC-LACE-07-01 | 19 Aug. 2016 |
| | HPMC; Conditions: 5 C., 25 C., 40 C. | | |
| | LDPE, no nitrogen purge, LDPE, | | |
| | pouched, N₂ overlaid, O₂ scavengers | | |
| 0226 | Stability on batch stored out of the pouch | AC-LACE-05-39 | 19 Aug. 2016 |
| | (5 C., 25 C.) - RS only | | |
| 0220 | Stability on LACE API (R&D Lot) | DG10_p100_060816 | 2 Sep. 2016 |
| | Conditions: −20 C., 5 C., 25 C. | | |

| Study # | Description of Formuiation | Time Points | Table | Discussion/Comments |
|---|---|---|---|---|
| N/A | No HPbCD, control formulation | 1 mo., 3 mo. | #13 | |
| | Dexolve, ~0.8 ME | 1 mo., 3 mo. | | |
| | 0.73 MEHPBCD | 1 mo., 3 mo. | | |
| 052516 | 15:1 ME HPBCD, 3.0% LACE-I | 1, 2, 8, 17, | #14 | Demonstrated |
| | | 20 days | | protective |
| | 3.0% LACE-I, no HPbCD | | | effect of |
| | | | | HPbCD |
| 0205 | 1:1 ME HPBCD, 3.0% LACE-I | 2 wk., | #15 | Stable at 5 C. |
| | 1:1 ME HPBCD, 1.92% LACE-I | 1, 2, 3 months | | Stable at 5 C. |
| | 1.5:1 ME HPBCD, 3.0% LACE-I | (3 months | | Stable at 5 C. |
| | 1.5:1 ME HPBCD, 1.92% LACE-I | on Oct. 1) | | Stable at 5 C. |
| 0214 | 1:1 ME HPbCD, 3.0% LACE-I | 2 wk, | #16 | Stable at 5 C., |
| | Cavasol HPbCD (better purity) | 1, 2, 3 months | | 25 C., and 40 C., |
| | | (3 months | | except Lipoic |
| | | on Oct. 12) | | Acid increases |
| | | | | at higher |
| | | | | temperatures |
| 0225 | 1:1 ME HPbCD, 3.0% LACE-I | 2 wk, | #17 | 1 month on |
| | Cavitron HPbCD (better purity) | 1, 3 months | | Sep. 19 |
| | 0.23% HPMC (Type 2910) | (3 months | | |
| | | on Nov. 19) | | |
| 0226 | 1:1 ME HPbCD, 3.0% LACE-I | 2 weeks, | | T0 data = 1 |
| | Cavasol HPbCD (better purity) | 1 month | | month @ 5 C., |
| | | (1 month | | inside the |
| | | on Sep. 12) | | pouch (from |
| | | | | Study# 0214) |
| 0220 | N/A | 3, 6, 12, | | in amber |
| | | 18, 24 mos. | | vials, |
| | | (3 months | | pouched |
| | | on Dec. 2) | | |

ME = Molar Equivalents

TABLE 13-1
FK-LACE-02-32: 1.92% LACE-Iodide Lot# 011510, Standard Formulation (no cyclodextrin)

| | Target | T0 | | T = 1 month @ 5° C. | | T = 1 month @ 25° C. | |
|---|---|---|---|---|---|---|---|
| Test | Specification | (RS: Apr. 8, 2016) | | (17 May 2016) | | (17 May 2016) | |
| Appearance | Clear, slightly | Complies | | Complies | | Complies | |
| | yellow solution | | | | | | |
| API Assay | 19.2 mg/g ± 10% | 15.44 mg/g | | 12.53 mg/g * | | 16.32 mg/g | |
| | (17.18-21.12) | | | | | | |
| API Related | Repost all | Oxid. LACE (2 pks.): | | Oxid LACE (2 pks.): | | Oxid. LACE (2 pks.): | |
| Substances | impurities >0.05% | RRT 0.48 | 0.13% | RRT 0.48 | 0.88% | RRT 0.45 | 2.78% |
| | | RRT 0.52 | 0.09% | RRT 0.52 | 0.47% | RRT 0.52 | 1.3290 |
| | | RRT 1.84 | 0.23% | RRT 3.84 | — | Lipoic Add (RP): 0.7% ** | |
| | | RRT 2.00 | 0.18% | RRT 2.00 | 0.32% | Rest are hidden by early- | |
| | | RRT 2.29 | 0.19% | Lipoic Acid(RP) 0.05% ** | | eluting Iodide peak due to | |
| | | Lipoic Acid(RP) <0.05% ** | | Total Imp. | 1.72% | Dexolve in previous sample. | |
| | | Total Imp. | 0.79% | | | Total Imp. | 4.80% |
| Associative | Report | 4.5% | | 36.2% | | 1.789% | |
| Species | (RP-HPLC method) | | | | | | |
| pH | 4 5 ± 0.5 | 4.66 | | 5.26 * | | 4.70 | |
| Osmolality | 300 | 279 | | 265 | | 287 | |
| (mOsm/kg) | (280-320) | | | | | | |

* May need to be repeated from 2^{nd} vial stored at 5° C., which has not been sampled as much.
** Lipoic Acid estimated from Area % of RRT 1.17 peal in RP-HPLC method used for Associative Species determination.

Due to repeated sampling and/or the storage conditions (lacking a foil bag with oxygen absorbers), this formulation shows some oxidative degradation.
As the Associative Species increased (1 month @5° C.), the Osmolality decreased

TABLE 13-2
FK-IACE-02-37: 1.92% LACE-Iodide lot# 011510, Formulation with 5% HP-JJ-CD (~0.75:1 mole ratio of HPBCD:LACE)

| | Target | T0 | | T = 1 month @ 5° C. | | T - 1 month @ 25° C. | | API (Lot# 011510) | |
|---|---|---|---|---|---|---|---|---|---|
| Test | Specification | (RS: Apr. 11, 2016) | | (17 May 2016) | | (17 May 2016) | | RS analyzed Mar. 22, 2016 | |
| Appearance | Clear, slightly | Complies | | Complies | | Complies | | Complies | |
| | yellow solution | | | | | | | | |
| API Assay | 19.2 mg/g ± 10% | 18.97 mg/g ** | | 17.54 mg/g | | 17.57 mg/g | | N/A | |
| | (17.18-21.12) | | | | | | | | |
| API Related | Report all | Oxid. LACE (3 pks.): | | Oxid. LACE (3 pks.): | | Oxid. LACE (2 pks.): | | Oxid. LACE (2 pks.): | |
| Substances | impurities >0.05% | RRT 0.40 | 0.05% | RRT 0.45 | 0.05% | RRT 0.48 | 1.77% | RRT 0.48 | 0.08% |
| | | RRT 0.48 | 0.04% | RRT 0.48 | 0.20% | RRT 0.52 | 0.53% | RRT 0.52 | 0.04% |
| | | RRT 0.52 | 0.02% | RRT 0.52 | 0.10% | RRT 2.00 | 0.50% | RRT 1.74 | 0.24% |

| RRT 2.00 | 0.46% | RRT 2.00 | 0.54% | Lipoic Acid (RP) 1.1% | | RRT 2.00 | 0.20% |
|---|---|---|---|---|---|---|---|
| Lipoic Acid(RP) <0.05% | | Lipoic Acid(RP) 0.1% | | Total Imp. | 3.90% | Total Imp. | 0.56% |
| Total Imp. | 0 57% | Total Imp. | 0.99% | | | | |

| | | | | | |
|---|---|---|---|---|---|
| Associative | Report | 0.0% | 0.0% (9 May 2016 - 27 days) | 0.18% | N/A |
| Species | (RP-HPLC method) | | | | |
| pH | 4.5 ± 0.5 | 4.78 | 5.39 *** | 4.81 | N/A |
| Osmolality | 300 | 297 | 306 | 310 | N/A |
| (mOsm/kg) | (280-320) | | | | |

** Previously reported 21.3 mg/g. Due to pump problems on HPLC causing a shift in retention times, the standard curve used in the earlier determination is now in question. Result reported here is based on current standard curve applied to 22 Apr. 2016 run.
*** May need to be repeated from 2^{nd} vial stored at 5° C., which has not been sampled as much.

### Comments

Despite repeated handling, the related substances in this lot have not substantially increased.
This compares favorably with the FK-LACE-02-32 batch (without cyclodextrin) which was placed on stability at the same time under the same storage conditions, and shows larger increases in oxidized LACE impurities at both 5° C. and 25° C.
This comparison indicates that the cyclodextrin may partially protect the LACE molecule from oxidation.

TABLE 14
Stabilization of LACE-Iodide by Hydroxypropyl Beta Cyclodextrin

| | 3.0% LACE-I, | Conditions: Stored at 25° C., LDPE Dropper bottles, | | | |
|---|---|---|---|---|---|
| AC-LACE- | 16.1% HPBCD | blanketed with N₂ initially, no pouching or oxygen scavenger | | | |
| 03-54 | Specifications | T = 24 h | | T = 48 h | |
| Related | Single unknown: | RRT 0.39 | 0.19% | RRT 0.39 | 0.19% |
| Substances | NMT 0.5% | RRT 0.51 | 0.10% | RRT 0.51 | 0.09% |
| | Total unknowns: | REIT 0.54 | 0.14% | RRT 0.54 | 0.14% |
| | NMT 2.0% | RRT 0.62 | 0.04% | RRT 0.62 | 0.04% |
| | Lipoic Acid: | RRT 0.67 | 0.03% | RRT 0.67 | 0.04% |
| | NMT 1.0% | Lipoate | 0.25% | Lipoate | 0.29% |
| | | RRT 1.55 | 0.11% | RRT 1.55 | 0.10% |
| | | Lipoic Acid | 0.30% | Lipoic Acid | 0.15% |
| | | Total Unk. | 0.61% | Total Unk. | 0.60% |
| | | Total Imp. | 1.16% | Total Imp. | 1.04% |

| | Conditions: Stored at 25° C., LDPE Dropper bottles, | | | | | |
|---|---|---|---|---|---|---|
| AC-LACE- | blanketed with N₂ initially, no pouching or oxygen scavenger | | | | | |
| 03-54 | T = 8 days | | T = 17 days | | T = 20 days | |
| Related | RRT 0.39 | 0.19% | RRT 0.39 | 0.12% | RRT 0.39 | 0.18% |
| Substances | RRT 0.51 | 0.39% | RRT 0.51 | 1.04% | RRT 0.51 | 1.49% |
| | RRT 0.54 | 0.05% | RRT 0.54 | 0.14% | RRT 0.54 | 0.31% |
| | RRT 0.62 | 0.04% | RRT 0.62 | 0.13% | RRT 0.62 | 0.20% |
| | RRT 0.67 | — | RRT 0.67 | — | RRT 0.67 | — |
| | Lipoate | 0.26% | Lipoate | 0.24% | Lipoate | 0.36% |
| | RRT 1.55 | 0.11% | RRT 1.55 | 0.11% | RRT 1.55 | 0.11% |
| | Lipoic Acid | 0.11% | Lipoic Acid | 0.11% | Lipoic Acid | 0.21% |
| | Total Unk. | 0.78% | Total Unk. | 1.54% | Total Unk. | 2.29% |
| | Total Imp. | 1.15% | Total Imp. | 1.89% | Total Imp. | 2.86% |

| | 3.0% LACE-I | Conditions: Stored at 25° C., LDPE Dropper bottles, | | | |
|---|---|---|---|---|---|
| AC-LACE- | NoHPBCD | blanketed with N₂ initially, no pouching or oxygen scavenger | | | |
| 03-56 | Specifications | T = 24 h | | T = 48 h | |
| Related | Single unknown: | RRT 0.39 | 0.36% | RRT 0.40 | 0.37% |
| Substances | NMT 0.5% | RRT 0.63 | 0.48% | RRT 0.57 | 0.11% |
| | Total unknowns: | RRT 0.71 | 0.14% | RRT 0.63 | 0.25% |
| | NMT 2.0% | RRT 0.76 | 0.17% | RRT 0.71 | 0.15% |
| | Lipoic Acid: | Lipoate | 0.18% | RRT 0.76 | 0.16% |
| | NMT 1.0% | RRT 1.55 | 0.14% | Lipoate | 0.21% |
| | | Lipoic Acid | 0.21% | RRT 1.59 | 0.13% |
| | | Total Unk. | 1.29% | RRT 1.69 | 0.11% |
| | | Total Imp. | 1.68% | Lipoic Acid | 0.17% |
| | | | | Total Unk. | 1.28% |
| | | | | Total Imp. | 1.66% |

| | Conditions: Stored at 25° C., LDPE Dropper bottles, | | | | | |
|---|---|---|---|---|---|---|
| AC-LACE- | blanketed with N₂ initially, no pouching or oxygen scavenger | | | | | |
| 03-56 | T = 8 days | | T = 17 days | | T = 20 days | |
| Related | RRT 0.41 | 0.35% | RRT 0.40 | 0.37% | RRT 0.41 | 0.26% |
| Substances | RRT 0.51 | 0.38% | RRT 0.50 | 1.60% | RRT 0.51 | 2.84% |
| | RRT 0.57 | 0.82% | RRT 0.52 | 0.52% | RRT 0.59 | 0.82% |
| | RRT 0.64 | 0.36% | RRT 0.55 | 1.10% | RRT 0.62 | 0.85% |
| | RRT 0.68 | 0.37% | RRT 0.61 | 0.81% | RRT 0.67 | — |
| | Lipoate | 0.17% | RRT 0.67 | 0.92% | Lipoate | 0.24% |
| | RRT 1.57 | 0.07% | Lipoate | 0.27% | RRT 1.45 | 0.21% |
| | Lipoic Acid | 0.28% | RRT 1.45 | 0.17% | RRT 1.52 | 0.51% |
| | Total Unk. | 2.35% | RRT 1.52 | 0.49% | RRT 1.67 | 0.05% |
| | Total Imp. | 2.80% | RRT 1.65 | 0.06% | Lipoic Acid | 0.06% |
| | | | Lipoic Acid | 0.07% | Total Unk. | 5.54% |
| | | | Total Unk. | 6.04% | Total Imp. | 5.84% |
| | | | Total Imp. | 6.38% | | |

TABLE 15-1
(Study #0205)
Effect of LACE-I concentration and different LACE-Iodide/HPBCD Molar Ratios

| | |
|---|---|
| Lot# | AC-LACE-05-21 |
| Description | 3% LACE-I/HPBCD |
| Molar Equivalents of API:HPbCD | 1:1 |
| API Lot# | |
| Container Closure | LDPE, Pouched, Oxygen Scavenger, N₂ overlay |
| Conditions | 5 C. |

Lot # AC-LACE-05-21, 5° C.; Start Bate: Jul. 1, 2016

| Test | Specification | T = 0 | | | 2 Week | 4 Weeks | 2 Month | 3 Month | API |
|---|---|---|---|---|---|---|---|---|---|
| Appearance | Clear, Light Yellow | PASS | | | PASS | PASS | PASS | | |
| pH | 4.5 (4.0-5.0) | 4.6 | | | — | — | — | | |
| Osmolality | 300 (280-320) mOsm | 303 | | | 307 | 310 | — | | |
| Associatvie | TBD | BLOQ | | | BLOQ | BLOQ | BLOQ | | |
| Species | | | | | | | | | |
| Viscosity | TBD | — | | | — | — | — | | |
| Assay, API | 30 (27-33) mg/g | 33.0 | | | 31.0 | 32.6 | — | | |
| Related | Report All | RRT | Impurity | Area % | Area % | Area % | Area % | Area % | Area % |
| Substances | Impurities > 0.05% | | | | | | | | |
| | | 0.51 | Oxidized | 0.06 | 0.12 | 0.21 | — | | 0.19 |
| | | 0.68 | Lace | 0.03 | 0.02 | 0.05 | | | 0.04 |
| | | 1.00 | API | 98.95 | 99.40 | 99.07 | | | 99.13 |
| | | 1.65 | | 0.26 | 0.29 | 0.38 | | | 0.34 |
| | | 1.86 | | 0.08 | 0.08 | 0.13 | | | 0.17 |
| | | 4.05 | Lipoic | 0.50 | ND | 0.06 | | | 0.13 |
| | | | Acid | | | | | | |

| | | | | |
|---|---|---|---|---|
| Total Impurities | 1.05 | 0.60 | 0.93 | 0.87 |

TABLE 15-2
(Study #0205)
Effect of LACE-I concentration and different LACE-Iodide/HP-B-CD Molar Ratios

| | |
|---|---|
| Lot# | AC-LACE-05-21 |
| Description | 3% LACE-I/HPBCD |
| Molar Equivalents of | 1:1 |
| API Lot# | |
| Container Closure | LDPE, Pouched, Oxygen Scavenger, N2 overlay |
| Conditions | 25 C. |

Lot # AC-LACE-05-21, 25° C.; Start Date: Jul. 1, 2016

| Test | Specification | T = 0 | | | 2 Week | 4 W eeks | 2 Month | 3 Month | API |
|---|---|---|---|---|---|---|---|---|---|
| Appearance | Clear, Light Yellow | PASS | | | PASS | PASS | PASS | | |
| pH | 4.5 (4.0-5.0) | 4.6 | | | — | — | — | | |
| Osmolality | 300 (280-320) mOsm | 303 | | | 308 | 304 | — | | |
| Associative | TBD | BLOQ | | | BLOQ | BLOQ | BLOQ | | |
| Specie | | | | | | | | | |
| Viscosity | TBD | — | | | — | — | — | | |
| Assay, API | 30 (27-33) mg/g | 33.0 | | | 30.8 | 32.5 | — | | |
| Related | Report All | RRT | Impurity | Area % | Area % | Area % | Area % | Area % | Area % |
| Substance | Impurities > 0.05% | | | | | | | | |
| | | 0.51 | | 0.06 | 0.17 | 0.29 | — | | 0.19 |
| | | 0.68 | Oxidized | 0.03 | 0.85 | 1.87 | | | 0.04 |
| | | | Lace | | | | | | |
| | | 1.00 | API | 98.95 | 98.07 | 96.62 | | | 99.13 |
| | | 1.65 | | 0.26 | 0.37 | 0.32 | | | 0.34 |
| | | 1.86 | | 0.08 | 0.26 | 0.31 | | | 0.17 |
| | | 4.05 | Lipoic | 0.50 | 0.18 | 0.39 | | | 0.13 |
| | | | Acid | | | | | | |

| | | | | |
|---|---|---|---|---|
| Total Impurities | 1.05 | 1.93 | 3.38 | 0.87 |

TABLE 15-3
(Study #0205)
Effect of LACE-1 concentration and different LACE-lodide/HPBCD Molar Ratios

| | |
|---|---|
| Lot# | AC-LACE-05-21 |
| Description | 3% LACE-I/HPBCD |
| Molar Equivalents of | 1:1 |
| API Lot# | |
| Container Closure | LDPE, Pouched, Oxygen |
| | Scavenger, N2 overlay |
| Conditions | 40 C. |

Lot # AC-LACE-05-21, 40° C.; Start Date: Jul. 1, 2016

| Test | Specification | T = 0 | | 2 Weeks | 4 Weeks | | 2 Months | 3 Months | API |
|---|---|---|---|---|---|---|---|---|---|
| Appearance | Clear, Light | PASS | | PASS | PASS | | PASS | | |
| | Yellow | | | | | | | | |
| pH | 4.5 (4.0-5.0) | 4.6 | | — | — | | — | | |
| Osmolality | 300 (280- | 303 | | 312 | 310 | | — | | |
| | 320) mOsm | | | | | | | | |
| Associative | TBD | BLOQ | | BLOQ | BLOQ | | BLOQ | | |
| Specie | | | | | | | | | |
| Viscosity | TBD | — | | — | — | | — | | |
| Assay, API | 30 (27-33) | 33.0 | | 30.8 | 32.1 | | — | | |
| | mg/g | | | | | | | | |
| | Report All | | | | | | | | |
| Related | Impurities > | | | | | | | | |
| Substances | 0.05% | RRT | Impurity | Area % | Area % | Area % | Area % | Area% | Area % |
| | | 0.51 | | 0.06 | 0.18 | 0.38 | | | 0.19 |
| | | 0.68 | Oxidized | 0.03 | 6.46 | 8.18 | | | 0.04 |
| | | | Lace | | | | | | |
| | | 1.00 | API | 98.85 | 91.97 | 89.23 | | | 99.13 |
| | | 1.65 | | 0.26 | 0.22 | 0.26 | | | 0.34 |
| | | 1.86 | | 0.08 | 0.32 | 0.50 | | | 0.17 |
| | | 4.05 | Lipoic | 0.50 | 0.65 | 1.32 | | | 0.13 |
| | | | Acid | | | | | | |

| | | | | |
|---|---|---|---|---|
| Total Impurities | 1.05 | 8.03 | 10.77 | 0.87 |

TABLE 15-4
(Study #0205)
Effect of LACE-I concentration and different LACE-Iodide/HPBCD Molar Ratios

| | |
|---|---|
| Lot# | AC-LACE-05-21B |
| Description | 1.92% LACE-I/HPBCD |
| Molar Equivalents of | 1:1 |
| API Lot# | |
| Container Closure | LDPE, Pouched, Oxygen |
| | Scavenger, N2 overlay |
| Conditions | 5 C. |

Lot # AC-LACE-05-21B, 5° C.; Start Date: Jul. 1, 2016

| Test | Specification | T = 0 | | | 2 Weeks | 4 Weeks | 2 Months | 3 Months | API |
|---|---|---|---|---|---|---|---|---|---|
| Appearance | Clear, Light | PASS | | | PASS | PASS | PASS | | |
| | Yellow | | | | | | | | |
| pH | 4.5 (4.0-5.0) | 4.5 | | | — | — | — | | |
| Osmolality | 300 (280- | 299 | | | 307 | 305 | — | | |
| | 320) mOsm | | | | | | | | |
| Associative | TBD | BLOQ | | | BLOQ | BLOQ | BLOQ | | |
| Specie | | | | | | | | | |
| Viscosity | TBD | — | | | — | — | — | | |
| Assay, API | 30 (27-33) | 20.5 | | | 19.4 | 20.8 | | | |
| | mg/g | | | | | | | | |
| | Report All | | | | | | | | |
| Related | Impurities > | | | | | | | | |
| Substances | 0.05% | RRT | Impurity | Area % | Area % | Area % | Area % | Area % | Area % |
| | | 0.51 | | 0.07 | 0.11 | 0.20 | — | | 0.19 |
| | | 0.53 | | ND | ND | 0.09 | | | |
| | | 0.68 | Oxidized | 0.0.3 | ND | 0.05 | | | 0.04 |
| | | | Lace | | | | | | |
| | | 1 | API | 99.35 | 99.37 | 99.01 | | | 99.13 |
| | | 1.65 | | 0.35 | 0.22 | 0.41 | | | 0.34 |
| | | 1.86 | | 0.09 | 0.13 | 0.19 | | | 0.17 |
| | | 2.28 | | ND | 0.08 | ND | | | |
| | | 4.05 | Lipoic | ND | 0.03 | ND | | | 0.13 |
| | | | Acid | | | | | | |

| | | | | |
|---|---|---|---|---|
| Total Impurities | 0.65 | 0.63 | 0.99 | 0.87 |

TABLE 15-5
(Study #0205)
Effect of LACE-I concentration and different LACE-Iodide/HPBCD Molar Ratios

| | |
|---|---|
| Lot# | AC-LACE-05-21B |
| Description | 1.92% LACE-I/HPBCD |
| Molar Equivalents of | 1:1 |
| API Lot# | |
| Container Closure | LDPE, Pouched, Oxygen |
| | Scavenger, N2 overlay |
| Conditions | 25 C. |

Lot# AC-LACE-05-21B, 25° C.; Start Date: Jul. 1, 2016

| Test | Specification | T = 0 | | | 2 Weeks | 4 Weeks | 2 Months | 3 Months | API |
|---|---|---|---|---|---|---|---|---|---|
| Appearance | Clear, Light | PASS | | | PASS | PASS | PASS | | |
| | Yellow | | | | | | | | |
| pH | 4.5 (4.0-5.0) | 4.5 | | | — | — | — | | |
| Osmolality | 300 (280- | 299 | | | 308 | 305 | — | | |
| | 320) mOsm | | | | | | | | |
| Associative | TBD | BLOQ | | | BLOQ | BLOQ | BLOQ | | |
| Specie | | | | | | | | | |
| Viscosity | TBD | — | | | — | — | — | | |
| Assay, API | 30 (27-33) | 20.5 | | | 19.5 | 20.7 | — | | |
| | mg/g | | | | | | | | |
| | Report All | | | | | | | | |
| Related | Impurities > | | | | | | | | |
| Substances | 0.05% | RRT | Impurity | Area % | Area % | Area % | Area % | Area % | Area % |
| | | 0.51 | | 0.07 | 0.12 | 0.24 | — | | 0.19 |
| | | 0.53 | | ND | ND | 0.11 | | | |
| | | 0.68 | Oxidized | 0.0.3 | 1.01 | 4.40 | | | 0.04 |
| | | | Lace | | | | | | |
| | | 1 | API | 99.35 | 97.91 | 94.04 | | | 99.13 |
| | | 1.65 | | 0.35 | 0.36 | 0.30 | | | 0.34 |
| | | 1.86 | | 0.09 | 0.31 | 0.47 | | | 0.17 |
| | | 2.28 | | ND | 0.09 | ND | | | |
| | | 4.05 | Lipoic | ND | 0.14 | 0.40 | | | 0.13 |
| | | | Acid | | | | | | |

| | | | | |
|---|---|---|---|---|
| Total Impurities | 0.65 | 2.08 | 5.96 | 0.87 |

TABLE 15-6
(Study #0205)
Effect of LACE-1 concentration and different LACE-lodide/HPBCD Molar Ratios

| | |
|---|---|
| Lot# | AC-LACE-05-21B |
| Description | 1.92% LACE-I/HPBCD |
| Molar Equivalents of | 1:1 |
| API Lot# | |
| Container Closure | LDPE, Pouched, Oxygen |
| | Scavenger, N2 overlay |
| Conditions | 4° C. |

Lot# AC-LACE-05-21B, 40° C.; Start Date: Jul. 1, 2016

| Test | Specification | T = 0 | | | 2 Weeks | 4 Weeks | 2 Months | 3 Months | API |
|---|---|---|---|---|---|---|---|---|---|
| Appearance | Clear, Light | PASS | | | PASS | PASS | PASS | | |
| | Yellow | | | | | | | | |
| pH | 4.5 (4.0-5.0) | 4.5 | | | — | — | — | | |
| Osmolality | 300 (280- | 299 | | | 312 | 308 | — | | |
| | 320) mOsm | | | | | | | | |
| Associative | TBD | BLOQ | | | BLOQ | BLOQ | BLOQ | | |
| Specie | | | | | | | | | |
| Viscosity | TBD | — | | | — | — | — | | |
| Assay, API | 30 (27-33) | 20.5 | | | 19.6 | 20.4 | — | | |
| | mg/g | | | | | | | | |
| | Report All | | | | | | | | |
| Related | Impurities > | | | | | | | | |
| Substances | 0.05% | RRT | Impurity | Area % | Area % | Area % | Area % | Area % | Area % |
| | | 0.51 | | 0.07 | 0.17 | 0.32 | — | | 0.19 |
| | | 0.53 | | ND | ND | 0.05 | | | |
| | | 0.68 | Oxidized | 0.03 | 14.01 | 16.95 | | | 0.04 |
| | | | Lace | | | | | | |
| | | 1 | API | 99.35 | 84.49 | 89.46 | | | 99.13 |
| | | 1.65 | | 0.35 | 0.28 | 0.28 | | | 0.34 |
| | | 1.86 | | 0.09 | 0.27 | 0.51 | | | 0.17 |
| | | 2.28 | | ND | 0.10 | ND | | | |
| | | 4.05 | Lipoic | ND | 0.67 | 1.27 | | | 0.13 |
| | | | Acid | | | | | | |

| | | | | |
|---|---|---|---|---|
| Total Impurities | 0.65 | 15.51 | 10.54 | 0.87 |

TABLE 15-7
(Study #0205)
Effect of LACE-I concentration and different LACE-lodide/HPBCD Molar Ratios

| | |
|---|---|
| Lot# | AC-LACE-05-23 |
| Description | 3% LACE-I/HPBCD |
| Molar Equivalents of | 1:1.5 |
| API Lot# | |
| Container Closure | LDPE, Pouched, Oxygen |
| | Scavenger, N2 overlay |
| Conditions | 5 C. |

Lot # AC-LACE-05-23, 5° C.; Start Date: Jul. 1, 2016

| Test | Specification | T = 0 | | | 2 Weeks | 4 Weeks | 2 Months | 3 Months | API |
|---|---|---|---|---|---|---|---|---|---|
| Appearance | Clear, Light | PASS | | | PASS | PASS | PASS | | |
| | Yellow | | | | | | | | |
| pH | 4.5 (4.0-5.0) | 4.5 | | | — | — | — | | |
| Osmolality | 300 (280- | 297 | | | 300 | 298 | — | | |
| | 320) mOsm | | | | | | | | |
| Associative | TBD | BLOQ | | | BLOQ | BLOQ | BLOQ | | |
| Specie | | | | | | | | | |
| Viscosity | TBD | — | | | — | — | — | | |
| Assay, API | 30 (27-33) | 32.0 | | | 32.2 | 32.2 | — | | |
| | mg/g | | | | | | | | |
| | Report All | | | | | | | | |
| Related | Impurities > | | | | | | | | |
| Substances | 0.05% | RRT | Impurity | Area % | Area % | Area % | Area % | Area % | Area % |
| | | 0.49 | | 0.05 | 0.09 | 0.18 | — | | 0.19 |
| | | 0.52 | | ND | ND | 0.06 | | | |
| | | 0.61 | Oxidized | 0.04 | 0.04 | 0.08 | | | 0.04 |
| | | | Lace | | | | | | |
| | | 1.00 | API | 99.10 | 99.29 | 98.45 | | | 99.13 |
| | | 1.52 | | 0.34 | 0.39 | 0.42 | | | 0.34 |
| | | 1.73 | | 0.07 | 0.08 | 0.17 | | | 0.17 |
| | | 4.11 | | 0.28 | ND | 0.06 | | | 0.13 |

| | | | | |
|---|---|---|---|---|
| Total Impurities | 0.68 | 0.71 | 1.55 | 0.87 |

TABLE 15-8
(Study #0205)
Effect of LACE-I concentration and different LACE-Iodide/HPBCD Molar Ratios

| | |
|---|---|
| Lot# | AC-LACE-05-23 |
| Description | 1.92% LACE-I/HPBCD |
| Molar Equivalents of | 1:1.5 |
| API Lot# | |
| Container Closure | LDPE, Pouched, Oxygen |
| | Scavenger, N2 overlay |
| Conditions | 25 C. |

Lot # AC-LACE-05-23, 25° C.; Start Date: Jul. 1, 2016

| Test | Specification | T = 0 | | | 2 Weeks | 4 Weeks | 2 Months | 3 Months | API |
|---|---|---|---|---|---|---|---|---|---|
| Appearance | Clear, Light | PASS | | | PASS | PASS | PASS | | |
| | Yellow | | | | | | | | |
| pH | 4.5 (4.0-5.0) | 4.6 | | | — | — | — | | |
| Osmolality | 300 (280- | 297 | | | 300 | 297 | — | | |
| | 320) mOsm | | | | | | | | |
| Associative | TBD | BLOQ | | | BLOQ | BLOQ | BLOQ | | |
| Specie | | | | | | | | | |
| Viscosity | TBD | — | | | — | — | — | | |
| Assay, API | 30 (27-33) | 32.0 | | | 32.2 | 32.2 | — | | |
| | mg/g | | | | | | | | |
| | Report All | | | | | | | | |
| Related | Impurities > | | | | | | | | |
| Substances | 0.05% | RRT | Impurity | Area % | Area % | Area % | Area % | Area % | Area % |
| | | 0.49 | | 0.05 | 0.09 | 0.21 | — | | 0.19 |
| | | 0.52 | | ND | ND | 0.07 | | | |
| | | 0.61 | Oxidized | 0.04 | 0.04 | 3.56 | | | 0.04 |
| | | | Lace | | | | | | |
| | | 1.00 | API | 99.10 | 99.29 | 95.00 | | | 99.13 |
| | | 1.52 | | 0.34 | 0.39 | 0.32 | | | 0.34 |
| | | 1.73 | | 0.07 | 0.08 | 0.30 | | | 0.17 |
| | | 4.11 | Lipoic | 0.28 | ND | 0.38 | | | 0.13 |
| | | | Acid | | | | | | |

| | | | | |
|---|---|---|---|---|
| Total Impurities | 0.68 | 0.71 | 5.00 | 0.87 |

TABLE 15-9
(Study #0205)
Effect of LACE-I concentration and different LACE-lodide/HPBCD Molar Ratios

| | |
|---|---|
| Lot# | AC-LACE-05-23 |
| Description | 3% LACE-1/HPBCD |
| Molar Equivalents of | 1:1.5 |
| API Lot# | |
| Container Closure | LDPE, Pouched, Oxygen |
| | Scavenger, N2 overlay |
| Conditions | 40 C. |

Lot # AC-LACE-05-23, 40° C.; Start Date: Jul. 1, 2016

| Test | Specification | T = 0 | | | 2 Weeks | 4 Weeks | 2 Months | 3 Months | API |
|---|---|---|---|---|---|---|---|---|---|
| Appearance | Clear, Light | PASS | | | PASS | PASS | PASS | | |
| | Yellow | | | | | | | | |
| pH | 4.5 (4.0-5.0) | 4.5 | | | — | — | — | | |
| Osmolality | 300 (280- | 297 | | | 301 | 299 | — | | |
| | 320) mOsm | | | | | | | | |
| Associative | TBD | BLOQ | | | BLOQ | BLOQ | BLOQ | | |
| Specie | | | | | | | | | |
| Viscosity | TBD | — | | | — | — | — | | |
| Assay, API | 30 (27-33) | 32.0 | | | 32.2 | 32.2 | — | | |
| | mg/g | | | | | | | | |
| | Report All | | | | | | | | |
| Related | Impurities > | | | | | | | | |
| Substances | 0.05% | RRT | Impurity | Area % | Area % | Area % | Area % | Area % | Area % |
| | | 0.49 | | 0.05 | 0.13 | 0.15 | — | | 0.19 |
| | | 0.61 | Oxidized | 0.04 | 27.07 | 21.07 | | | 0.04 |
| | | | Lace | | | | | | |
| | | 1.00 | API | 99.10 | 71.56 | 76.39 | | | 99.13 |
| | | 1.52 | | 0.34 | 0.23 | 0.29 | | | 0.34 |
| | | 1.73 | | 0.07 | 0.41 | 0.52 | | | 0.17 |
| | | 4.11 | Lipoic | 0.28 | 0.48 | 1.18 | | | 0.13 |
| | | | Acid | | | | | | |

| | | | | |
|---|---|---|---|---|
| Total Impurities | 0.68 | 2.13 | 23.61 | 0.87 |

TABLE 16-1
(Study #0214)
“No Nitrogen” Processing of LACE-Iodide in HPBCD:
Effect on Stability

| | |
|---|---|
| Lot# | AC-LACE-05-3 9 |
| Description | 3% LACE-I/HPBCD |
| Molar Equivalents of | 1:1 |
| API Lot# | |
| Container Closure | LDPE, Pouched. Oxygen |
| | Scavenger, N2 overlay |
| Conditions | 5 C. |

Lot # AC-LACE-05-39, 5° C.; Start Date: Jul. 13, 2016

| Test | Specification | T = 0 | 2 Week | | 1 Month | 2 Month | 3 Month | API |
|---|---|---|---|---|---|---|---|---|
| Appearance | Clear, Light Yellow | PASS | PASS | | PASS | | | |
| pH | 4.5 (4.0-5.0) | 4.7 | — | | | | | |
| Osmolality | 300 (280-320) mOsm | 304 | 302 | | 298 | | | |
| Associative Species | TBD | BLOQ | BLOQ | | BLOQ | | | |
| Viscosity | TBD | — | — | | — | | | |
| Assay, API | 30 (27-33) mg/g | 31.6 | 32.7 | | 31.3 | | | |
| | Report All | | | | | | | |
| Related | Impurities > | | | | | | | |
| Substances | 0.05% | RRT | Impurity | Area % | Area % | Area % | Area % | Area % |
| | | 0.51 | API | 0.08 | 0.16 | 0.09 | | 0.10 |
| | | 0.53 | | 0.03 | ND | 0.03 | | ND |
| | | 0.68 | | ND | ND | 0.04 | | 0.04 |
| | | 0.93 | | ND | ND | 0.05 | | ND |
| | | 1.00 | | 99.26 | 99.19 | 99.19 | | 99.13 |
| | | 1.36 | | 0.10 | ND | 0.05 | | ND |
| | | 1.65 | | 0.39 | 0.42 | 0.42 | | 0.34 |
| | | 1.86 | | 0.04 | 0.09 | 0.07 | | ND |
| | | 2.28 | | ND | 0.08 | 0.07 | | 0.17 |
| | | 4.05 | Lipoic | ND | ND | 0.06 | | 0.13 |
| | | | Acid | | | | | |

| | | | | |
|---|---|---|---|---|
| Total Impurities | 0.74 | 0.81 | 0.81 | 0.87 |

TABLE 16-2
(Study #0214)
“No Nitrogen” Processing of LACE-Iodide in HPBCD:
Effect on Stability

| | |
|---|---|
| Lot# | AC-LACE-05-39 |
| Description | 3% LACE-I/HPBCD |
| Molar Equivalents of | 1:1 |
| API Lot# | LDPE, Pouched, Oxygen |
| | Scavenger, N2 overlay |
| Container Closure | |
| Conditions | 25 C. |

Lot # AC-LACE-05-39, 25° C.; Start Date: Jul. 13, 2016

| Test | Specification | T = 0 | 2 Week | | 1 Month | 2 Month | 3 Month | API |
|---|---|---|---|---|---|---|---|---|
| Appearance | Clear, Light Yellow | PASS | PASS | | PASS | | | |
| pH | 4.5 (4.0-5.0) | 4.7 | — | | | | | |
| Osmolality | 300 (280-320) mOsm | 304 | 300 | | 299 | | | |
| Associative Species | TBD | BLOQ | BLOQ | | BLOQ | | | |
| Viscosity | TBD | | — | | — | | | |
| Assay, API | 30 (27-33) mg/g | 31.6 | 33.3 | | 30.3 | | | |
| | Report All | | | | | | | |
| Related | Impurities > | | | | | | | |
| Substances | 0.05% | RRT | Impurity | Area % | Area % | Area % | Area % | Area % |
| | | 0.51 | API | 0.08 | 0.20 | 0.17 | | 0.10 |
| | | 0.53 | Lipoic | 0.03 | 0.04 | 0.04 | | ND |
| | | 0.68 | Acid | ND | ND | 0.06 | | 0.04 |
| | | 0.93 | | ND | ND | 0.04 | | ND |
| | | 1.00 | | 99.26 | 99.82 | 98.46 | | 99.13 |
| | | 1.23 | | ND | ND | 0.25 | | ND |
| | | 1.36 | | 0.10 | 0.08 | 0.06 | | 0.34 |
| | | 1.65 | | 0.39 | 0.41 | 0.40 | | ND |
| | | 1.86 | | 0.04 | 0.11 | ND | | 0.17 |
| | | 2.28 | | ND | 0.09 | 0.09 | | 0.13 |
| | | 4.05 | | ND | 0.21 | 0.43 | | |

| | | | | |
|---|---|---|---|---|
| Total Impurities | 0.74 | 1.18 | 1.54 | 0.87 |

TABLE 16-3
(Study #0214)
“No Nitrogen” Processing of LACE-Iodide in HPBCD:
Effect on Stability

| | |
|---|---|
| Lot# | AC-LACE-05-39 |
| Description | 3% LACE-I/HPBCD |
| Molar Equivalents of | 1:1 |
| API Lot# | |
| Container Closure | LDPE, Pouched, Oxygen |
| | Scavenger, N2 overlay |
| Conditions | 4° C. |

Lot # AC-LACE-05-39, 40° C.; Start Date: Jul. 13, 2016

| Test | Specification | T = 0 | 2 Week | | 1 Month | 2 Month | 3 Month | API |
|---|---|---|---|---|---|---|---|---|
| Appearance | Clear, Light Yellow | PASS | PASS | | PASS | | | |
| pH | 4.5 (4.0-5.0) | 4.7 | — | | | | | |
| Osmolality | 300 (280-320) mOsm | 304 | 302 | | 302 | | | |
| Associative Species | TBD | BLOQ | BLOQ | | BLOQ | | | |
| Viscosity | TBD | — | — | | — | | | |
| Assay, API | 30 (27-33) mg/g | 31.6 | 33.7 | | 31.1 | | | |
| | Report All | | | | | | | |
| Related | Impurities > | | | | | | | |
| Substances | 0.05% | RRT | Impurity | Area % | Area % | Area % | Area % | Area % |
| | | 0.51 | API | 0.08 | 0.36 | 0.20 | | 0.10 |
| | | 0 53 | Lipoic | 0.03 | ND | ND | | ND |
| | | 0.68 | Acid | ND | ND | 0.12 | | 0.04 |
| | | 0.93 | | ND | ND | ND | | ND |
| | | 1.00 | | 99.26 | 97.93 | 97.19 | | 99.13 |
| | | 1.36 | | 0.10 | 0.11 | 0.33 | | ND |
| | | 1.65 | | 0.39 | 0.32 | ND | | 0.34 |
| | | 1.86 | | 0.04 | 0.18 | 0.29 | | ND |
| | | 2.28 | | ND | 0.06 | 0.08 | | 0.17 |
| | | 4.05 | | ND | 1.01 | 1.80 | | 0.13 |

| | | | | |
|---|---|---|---|---|
| Total Impurities | 0.74 | 2.08 | 2.81 | 0.87 |

TABLE 17-1
(Study #0225)
LACE-Iodide Formulation in Cavitron HPBCD and 0.23% HPMC
Effect on Stability

| | |
|---|---|
| Lot# | AC-LACE-07-01 |
| Description | 3% LACE-I/HPBCD |
| Molar Equivalents of | 1:1 |
| API Lot# | |
| Container Closure | LDPE, Pouched, Oxygen |
| | Scavenger, N2 overlay |
| Conditions | 5 C. |

Lot # AC-LACE 05-39, 40° C.; Start Date: Jul. 13, 2016

| Test | Specification | T = 0 | 2 Week | | 1 Month | 3 Month | API |
|---|---|---|---|---|---|---|---|
| Appearance | Clear, Light Yellow | PASS | PASS | | PASS | | |
| pH | 4.5 (4.0-5.0) | 4.6 | — | | | | |
| Osmolality | 300 (280-320) mOsm | 280 | — | | | | |
| Associative Species | TBD | | — | | | | |
| Viscosity | TBD | 10 | — | | | | |
| Assay, API | 30 (27-33) mg/g | 28.6 | 29.4 | | | | |
| | Report All | | | | | | |
| Related | Impurities > | | | | | | |
| Substances | 0.05% | RRT | Impurity | Area % | Area % | Area % | Area % |
| | | 0.50 | API | 0.04 | 0.12 | | 0.20 |
| | | 0.59 | Lipoic | 0.07 | 0.09 | | 0.04 |
| | | 0.74 | Acid | 0.02 | 0.04 | | |
| | | 0.86 | | 0.08 | 0.04 | | |
| | | 0.91 | | 0.02 | ND | | |
| | | 3.00 | | 99.15 | 99.18 | | 99.13 |
| | | 1.13 | | 0.08 | 0.15 | | |
| | | 1.27 | | 0.38 | 0.32 | | 0.34 |
| | | 1.42 | | 0.07 | 0.08 | | 0.17 |
| | | 3.07 | | ND | ND | | 0.13 |

| | | | |
|---|---|---|---|
| Total Impurities | 0.85 | 0.82 | 0.87 |

TABLE 17-2
(Study #0225)
LACE-lodide Formulation in Cavitron HPBCD and
0.23% HPMC Effect on Stability

| | |
|---|---|
| Lot# | AC-LACE-07-01 |
| Description | 3% LACE-I/HPBCD |
| Molar Equivalents of | 1:1 |
| API Lot# | |
| Container Closure | LDPE, Pouched, Oxygen |
| | Scavenger, N2 overlay |
| Conditions | 25 C. |

Lot # AC-LACE-07-01, 25° C.; Start Date: Aug. 19, 2016

| Test | Specification | T = 0 | 2 Week | | 1 Month | 3 Month | API |
|---|---|---|---|---|---|---|---|
| Appearance | Clear, Light Yellow | PASS | | | | | |
| pH | 4.5 (4.0-5.0) | 4.6 | | | | | |
| Osmolality | 300 (280-320) mOsm | 280 | | | | | |
| Associative Species | JBD | | | | | | |
| Viscosity | TBD | 10 | | | | | |
| Assay, API | 30 (27-33) mg/g | 28.6 | | | | | |
| | Report All | | | | | | |
| Related | Impurities > | | | | | | |
| Substances | 0.05% | RRT | Impurity | Area % | Area % | Area % | Area % |
| | | 0.50 | API | 0.04 | 0.15 | | 0.20 |
| | | 0.59 | | 0.07 | 0.10 | | 0.04 |
| | | 0.74 | | 0.02 | 0.02 | | |
| | | 0.86 | | 0.08 | 0.025 | | |
| | | 0.91 | | 0.02 | ND | | |
| | | 1.00 | | 99.15 | 98.93 | | 99.13 |
| | | 1.33 | | 0.08 | ND | | |
| | | 1.27 | | 0.38 | 0.49 | | 0.34 |
| | | 1.42 | Lipoic | 0.07 | 0.07 | | 0.17 |
| | | | Acid | ND | 0.21 | | 0.13 |

| | | | |
|---|---|---|---|
| Total Impurities | 0.85 | 1.07 | 0.87 |

TABLE 17-3
(Study #0225)
LACE-lodide Formulation in Cavitron HPBCD and 0.23% HPMC
Effect on Stability

| | |
|---|---|
| Lot# | AC-LACE-07-01 |
| Description | 3% LACE-I/HPBCD |
| Molar Equivalents of | 1:1 |
| API Lot# | |
| Container Closure | LDPE, Pouched, Oxygen |
| | Scavenger, N2 overlay |
| Conditions | 4° C. |

Lot # AC-LACE-97-01, 40° C.; Start Date: Aug. 19, 2016

| Test | Specification | T = 0 | 2 Week | | 1 Month | 3 Month | API |
|---|---|---|---|---|---|---|---|
| Appearance | Clear, Light Yellow | PASS | | | | | |
| pH | 4.5 (4.0-5.0) | 4.6 | | | | | |
| Osmelality | 300 (280-320) mOsm | 280 | | | | | |
| Associative Species | I&C | | | | | | |
| Viscosity | BD | 10 | | | | | |
| Assay, APL | 30 (27-33) mg/g | 28.6 | | | | | |
| | Report All | | | | | | |
| Related | Impurities > | | | | | | |
| Substances | 0.05% | RRT | Impurity | Area % | Area % | Area % | Area % |
| | | 0.50 | APE | 0.04 | 0.19 | | 0.20 |
| | | 0.59 | | 0.07 | 0.14 | | 0.04 |
| | | 0.74 | | 0.02 | ND | | |
| | | 0.86 | | 0.08 | ND | | |
| | | 0.91 | | 0.02 | ND | | |
| | | 1.00 | | 99.15 | 98.19 | | 99.13 |
| | | 1.13 | | 0.08 | 0.20 | | |
| | | 1.27 | | 0.38 | 0.37 | | 0.34 |
| | | 1.42 | | 0.07 | 0.08 | | 0.17 |
| | | 3.07 | Lipoic | | 0.84 | | 0.13 |
| | | | Acid | | | | |

| | | | |
|---|---|---|---|
| Total Impurities | 0.85 | 1.82 | 0.87 |

TABLE 18-1
(Study #0226)
LACE-Iodide Formulation in Cavitron HPBCD in
Molar Ratto 1:1 (Stored with no oxygen protection (no
O2 scavengers, no N2 overlay, no pouch)
Effect on Stability

| | |
|---|---|
| Lot# | AC-LACE-05-39 |
| Description | 3% LACE-I/HPBCD |
| Molar Equivalents of | 1:1 |
| APT Lot# | |
| Container Closure | LDPE, no O2 scavengers, no |
| | N2 overlay, no pouch |
| Conditions | 5 C. |

Lot # AC-LACE-05-39, 5° C.; Removed O2 Scavengers: Aug. 19, 2016

| | | | T0 | | | CONTROL | |
|---|---|---|---|---|---|---|---|
| | | Initial | (With O2 | 2 Weeks | 4 Weeks | (with | |
| | | Bottling | Scavengers 1 | (No O2 | (No O2 | O2 | |
| Test | Specification | Jul. 13, 2016 | Months) | Scavengers) | Scavengers) | Scavengers 2 | API |
| Appearance | Clear, Light Yellow | PASS | PASS | PASS | | | |
| pH | 4.5 (4.0-5.0) | 4.7 | | — | | | |
| Osmolality | 300 (280-320) mOsm | 304 | 298 | — | | | |
| Associative Species | TBD | BLOQ | BLOQ | — | | | |
| Viscosity | TBD | — | — | — | | | |
| Assay, API | 30 (27-33) mg/g | 31.6 | 31.3 | — | | | |

| | Report All | | | | | | |
|---|---|---|---|---|---|---|---|
| Related | Impurities > | | | | | | |
| Substances | 0.05% | RRT | Impurity | Area % | Area % | Area % | Area % |
| | | 0.51 | | 0.08 | 0.09 | 0.13 | 0.10 |
| | | 0.53 | | 0.03 | 0.03 | 0.04 | ND |
| | | 0.68 | | ND | 0.04 | 0.06 | 0.04 |
| | | 0.93 | | ND | 0.05 | ND | ND |
| | | 1.00 | API | 99.6 | 99.19 | 98.78 | 99.13 |
| | | 1.2 | | ND | ND | 0.105 | ND |
| | | 1.36 | | 0.10 | 0.05 | 0.39 | ND |
| | | 1.65 | | 0.39 | 0.42 | 0.35 | 0.34 |
| | | 1.86 | | 0.04 | 0.07 | ND | ND |
| | | 2.28 | | ND | 0.07 | ND | 0.17 |
| | | 4.05 | Lipoic | ND | 0 06 | 0.15 | 0.13 |
| | | | Acid | | | | |

| | | | | |
|---|---|---|---|---|
| Total Impurities | 0.74 | 0.81 | 1.22 | 0.87 |

TABLE 18-2
(Study #0226)
LACE-Iodide Formulation in Cavitron HPBCD in
Molar Ratio 1:1 (Stored with no oxygen protection (no
O2 scavengers, no N2 overlay, no pouch)
Effect on Stability (5<, 25<)

| | |
|---|---|
| Lot# | AC-LACE-05-39 |
| Description | 3% LACE-I/HPBCD |
| Molar Equivalents of | 1:1 |
| API Lot# | |
| Container Closure | LDPE, no O2 scavengers, no |
| | N2 overlay, no pouch |
| Conditions | 25 C. |

Lot # AC-LACE-05-39, 25° C.; Removed O2 Scavengers: Aug. 19, 2016

| | | | T0 | | | CONTROL | |
|---|---|---|---|---|---|---|---|
| | | Initial | (With O2 | 2 Weeks | 4 Weeks | (with O2 | |
| | | Bottling | Scavengers 1 | (No O2 | (No O2 | Scavengers | |
| Test | Specification | Jul. 13, 2016 | Months) | Scavengers) | Scavengers) | 2 Months | API |
| Appearance | Clear, Light Yellow | PASS | PASS | PASS | | | |
| pH | 4.5 (4.0-5.0) | 4.7 | | — | | | |
| Osmolality | 300 (280-320) mOsm | 304 | 298 | — | | | |
| Associative Species | TBD | BLOQ | BLOQ | — | | | |
| Viscosity | TBD | — | — | — | | | |
| Assay, API | 30 (27-33) mg/s | 31.6 | 31.3 | — | | | |

| | Report All | | | | | | |
|---|---|---|---|---|---|---|---|
| Related | Impurities > | | | | | | |
| Substances | 0.05% | RRT | Impurity | Area % | Area % | Area % | Area % |
| | | 0.51 | | 0.08 | 0.09 | 0.37 | 0.10 |
| | | 0.53 | | 0.03 | 0.03 | 0.16 | ND |
| | | 0.68 | | ND | 0.04 | 0.19 | 0.04 |
| | | 0.93 | | ND | 0.05 | ND | ND |
| | | 1.00 | API | 99.26 | 99.19 | 98.47 | 99.13 |
| | | 1.2 | | ND | ND | 0.15 | ND |
| | | 1.36 | | 0.10 | 0.05 | 0.38 | ND |
| | | 1.65 | | 0.39 | 0.42 | ND | 0.34 |
| | | 1.86 | | 0.04 | 0.07 | ND | ND |
| | | 2.28 | | ND | 0.07 | ND | 0.17 |
| | | 4.05 | Lipoic | ND | 0.06 | 0.29 | 0.13 |
| | | | Acid | | | | |

| | | | | |
|---|---|---|---|---|
| Total impurities | 0.74 | 0.81 | 1.53 | 0.87 |

### Example 19

### Method of Formulation for LACE-Iodide Drug Product Solution

### General Process Sequence

### LAC-I/HPbCD (No HPMC)

1. Into a beaker add in order: WFI, alanine, glycerol, HP-B-CD, and Benzalkonium Chloride solution (BAK 0.005 g/mL in WFI).
2. Place beaker on magnetic stirrer to combine excipients.
3. Adjust pH using 1 N HCl, target pH 4.5
4. Place beaker into jacketed vessel hooked up to water heater/chiller circulator set to 25° C. (add distilled water to jacketed vessel for thermal conductivity). Immerse Scilogix mixing paddle and stir at approximately 500 RPM.
5. Add API in small increments while stirring. Upon completion of the addition of the API, allow formulation to stir for 45-60 minutes to ensure complete dissolution,
6. Remove beaker from mixing apparatus and weigh. Add WFI for account for any loss due to evaporation.
7. Filter formulation (0.2 uM PVDF).
   LACE-I With 0.23% HPMC (two solution process)

A. Solution 1-1.16% (w/w) Hypromeilose 2910 solution in WFI
1. Into a beaker add WH.
2. Place beaker into jacketed vessel hooked up to water heater/chiller circulator set to 90 ° C. (add distilled water to jacketed vessel for thermal conductivity). Immerse Sciolgex mixing paddle and stir at approximately 400 RPM.
3. Once WFI is 2:70° C., begin adding Hypromellose 2910 to disperse. Increase mixing speed to 650 RPM.
4. Once all HPMC has been added, reduce temperature of heater/chiller water circulator to 10° C. and continue to mix.
5. When solution has cooled and become clear and viscous, remove beaker from mixing apparatus and weigh. Add WFI for account for any loss due to evaporation.

B. Solution 2—LACE-Formulation without HPMC
1. into a beaker add in order: WFI, alanine, glycerol, HP-13-CD

2. Place beaker into jacketed vessel hooked up to water heater/chiller circulator set to 25 ° C. (add distilled water to jacketed vessel for thermal conductivity). Immerse Sciolgex mixing paddle and stir at approximately 500 RPM.
3. Adjust pH using 1 N HCl to 4.18
4. Add API in small increments while stirring. Upon completion of the addition of the API, allow formulation to stir for 45-60 minutes to ensure complete dissolution.
5. Add BAK solution (BAK 0.005 g/mL in WFI).
6, Remove beaker from mixing apparatus and weigh. Add WFI for account for any loss due to evaporation.
7. Measure pH and adjust if necessary.

C. Combine Solutions I and 2
1. Weigh out a designated portion of Solution 1 into a beaker.
2. Place beaker into jacketed vessel hooked up to water heater/chiller circulator set to 25 ° C. (add distilled water to jacketed vessel for thermal conductivity). Immerse Sciolgex mixing paddle and stir at approximately 130 RPM.
3. Add solution 2 into solution 1 while mixing.
4. Remove beaker from mixing apparatus.
5. Sterile filter using 0.2 μM PVDF filter.

1. A stable and biocompatible composition of matter for the treatment of presbyopia comprising a pharmaceutical salt of 0.1-10% lipoic acid choline ester, 1-30% of a cyclodextrin, 0.1-2% of a tonicity adjusting agent, 0.1-0.5% of a viscosity enhancement agent, 0.05% to about 1.0% of a biochemical energy source and water for injection.

2. The composition of claim 1, wherein the cyclodextrin comprises hvdroxypropyl beta cyclodextrin in the concentration range 0.1.-0.5%.

3. The composition of claim 2, wherein the tonicity adjusting agent comprises glycerol or sodium chloride.

4. (canceled)

5. The composition of claim 1, further comprising a stabilizer selected from the group consisting of methionine, cysteine and histidine.

6. The composition of claim 5, further comprising benzalkonium chloride as the preservative.

7. The composition of claim 6, comprising alanine as the biochemical energy source.

8. The composition of claim 1, wherein the pharmaceutical salt of the lipoic acid choline ester is a chloride or an iodide.

9. The composition of any one of claim 1, wherein the composition is preservative free.

10. A method of producing the stable and biocompatible pharmaceutical composition according to claim 1, comprising:
A. finely grinding the lipoic acid choline ester,
B. adding the lipoic acid acid choline ester, cyclodextrin, tonicity adjusting agent, viscosity enhancement agent, biochemical energy source, and optionally a preservative to water that is de-oxygenated to less than 5 ppm with an inert gas to form a component mixture
C. vigorously mixing the component mixture at room temperature
D. filling ophthalmic bottles with the component mixture
E. packaging the filled-and-capped ophthalmic bottles in gas-impermeable foil pouches, said pouches containing an oxy gen scavenger, and an inert gas,
F. storing the packages at 2-8C.

11. The method of claim 10, in which the component mixture pH is adjusted to a pH range of 4-5.

12. The method of claim 10, in which the mixing is performed under a nitrogen blanket or under ambient air.

13. (canceled)

14. The method of claim 10, in which the final package contains a nitrogen overlay.

15. The method of claim 10, in which the lipoic acid choline ester is ground into finely divided powder of an average size of 5 mm or less.

16. The method of claim 10, in which the deoxygenation level is 2 ppm.

17. The method of claim 10, in which temperature of mixing is between 20-25C.

18. The method of claim 10, in which the components are mixed for 8 hours.

19. The method of claim 10, in which the inert gas is nitrogen.

20. The method of claim 10, in which the ophthalmic bottle is Type 1 pharmaceutical glass, HDPE, PP, LDPE, PET or PTFE.

21. The method of claim 10, wherein the ophthalmic bottle is a blow-fill-seal unit.

22. The method of claim 10, wherein the ophthalmic bottle is a multi-dose unit.

23. (canceled)

24. (canceled)
